(19) United States
(12) Patent Application Publication
(10) Pub. No.: US 2021/0179574 A1
(43) Pub. Date: Jun. 17, 2021

(54) ELECTROCHEMICAL, BROMINATION, AND OXYBROMINATION SYSTEMS AND METHODS TO FORM PROPYLENE OXIDE OR ETHYLENE OXIDE

(71) Applicant: Calera Corporation, Moss Landing, CA (US)

(72) Inventors: Kyle Self, San Jose, CA (US); Michael Joseph Weiss, Los Gatos, CA (US); Ryan J. Gilliam, San Jose, CA (US); Thomas Albrecht, Santa Clara, CA (US); Gal Mariansky, Morgan Hill, CA (US)

(21) Appl. No.: 17/123,707

(22) Filed: Dec. 16, 2020

Related U.S. Application Data
(63) Continuation-in-part of application No. 16/804,665, filed on Feb. 28, 2020.
(60) Provisional application No. 62/948,459, filed on Dec. 16, 2019.

(51) Int. Cl. C07D 301/27; C25B 3/29; C25B 3/07; C07C 29/64; C07D 303/04; C25B 3/27

(52) U.S. Cl. CPC C07D 301/27; C25B 3/29; C25B 3/27; C07C 29/64; C07D 303/04; C25B 3/07

(57) ABSTRACT

Disclosed herein are methods and systems that relate to various configurations of electrochemical, bromination, oxybromination, bromine oxidation, hydrolysis, neutralization, and epoxidation reactions to form propylene bromohydrin, propanal, and propylene oxide or to form bromoethanol, bromoacetaldehyde, and ethylene oxide.

### CROSS-REFERENCE TO RELATED APPLICATIONS

This application is a continuation-in-part of U.S. patent application Ser. No. 16/804,665, filed Feb. 28, 2020, and claims benefit of U.S. Provisional Application No. 62/948,459, filed Dec. 16, 2019, both of which are incorporated herein by reference in their entireties in the present disclosure.

### BACKGROUND

Polyurethane production remains one of the most environmentally challenging manufacturing processes in industrial polymerization. Formed from addition reactions of di-isocyanates and polyols, polyurethanes may have a significant embedded environmental footprint because of the challenges associated with both feedstocks. Polyols are themselves polymerization derivatives which use propylene oxide as raw materials. Traditionally, propylene oxide (PO) may be synthesized from a chlorinated intermediate, propylene chlorohydrin.

Ethylene oxide may be one of the important raw materials used in large-scale chemical production. Most ethylene oxide may be used for synthesis of ethylene glycols, including diethylene glycol and triethylene glycol, that may account for up to 75% of global consumption. Other important products may include ethylene glycol ethers, ethanolamines and ethoxylates. Among glycols, ethylene glycol may be used as antifreeze, in the production of polyester and polyethylene terephthalate (PET—a raw material for plastic bottles), liquid coolants and solvents.

However, environmentally acceptable processes for the economic production of propylene oxide and ethylene oxide remain elusive. High costs of chlorine and significant waste water production (approximately 40 tonnes of waste water per tonne of PO) has caused manufacturers to look for process options with reduced environmental and safety risks.

### SUMMARY

There are provided methods and systems herein that relate to environmentally friendly and low cost production of propylene oxide (PO) and ethylene oxide (EO) and other commercially valuable products, such as, but not limited to, propanal and bromoacetaldehyde.

In one aspect, there are provided methods, comprising:

brominating propylene with an aqueous medium comprising metal bromide with metal ion in higher oxidation state, metal bromide with metal ion in lower oxidation state, and saltwater to result in one or more products comprising dibromopropane (DBP) and propylenebromohydrin (PBH) and reduction of the metal bromide with the metal ion in the higher oxidation state to the metal bromide with the metal ion in the lower oxidation state;

epoxidizing the one or more products comprising DBP and PBH with a base to form propylene oxide (PO) and unreacted DBP; and

subjecting the unreacted DBP to hydrolysis under one or more reaction conditions to result in hydrolysis products comprising PBH and propanal.

In one aspect, there are provided methods, comprising:

brominating propylene with an aqueous medium comprising metal bromide with metal ion in higher oxidation state, metal bromide with metal ion in lower oxidation state, and saltwater to result in one or more products comprising dibromopropane (DBP) and reduction of the metal bromide with the metal ion in the higher oxidation state to the metal bromide with the metal ion in the lower oxidation state;

subjecting the DBP to hydrolysis under one or more reaction conditions to result in hydrolysis products comprising PBH and propanal;

epoxidizing the hydrolysis products comprising PBH and propanal with a base to form propylene oxide (PO) and unreacted propanal.

In some embodiments of the aforementioned aspects, the one or more reaction conditions in the hydrolysis reaction comprise organic:aqueous ratio between 0.5:10-10:0.5.

In some embodiments of the aforementioned aspects and embodiments, the one or more reaction conditions in the hydrolysis reaction comprise Lewis acid selected from the group consisting of silicon bromide; germanium bromide; tin bromide; boron bromide; aluminum bromide; gallium bromide; indium bromide; thallium bromide; phosphorus bromide; antimony bromide; arsenic bromide; copper bromide; zinc bromide; titanium bromide; vanadium bromide; chromium bromide; manganese bromide; iron bromide; cobalt bromide; nickel bromide; lanthanide bromide; and triflate.

In some embodiments of the aforementioned aspects and embodiments, the method further comprises separating the one or more products comprising PBH and DBP from the aqueous medium, before subjecting the one or more products comprising PBH and DBP to the epoxidation reaction.

In some embodiments of the aforementioned aspects and embodiments, the method further comprises without separating subjecting the aqueous medium comprising metal bromide with metal ion in higher oxidation state, metal bromide with metal ion in lower oxidation state, and saltwater, and the one or more products comprising PBH and DBP, to the hydrolysis reaction before the epoxidation reaction (followed by the hydrolysis reaction).

In some embodiments of the aforementioned aspects and embodiments, the hydrolysis products further comprise bromopropanal, dibromopropanal, or combinations thereof.

In some embodiments of the aforementioned aspects and embodiments, the hydrolysis products further comprise acetone, bromoacetone, dibromoacetone, or combinations thereof.

In some embodiments of the aforementioned aspects and embodiments, the hydrolysis products further comprise unreacted DBP.

In some embodiments of the aforementioned aspects and embodiments, the method further comprises circulating the hydrolysis products comprising PBH and propanal from the hydrolysis reaction back to the epoxidation reaction to form the PO, the unreacted DBP, unreacted propanal, or combinations thereof.

In some embodiments of the aforementioned aspects and embodiments, the method further comprises separating the PO from the unreacted propanal to isolate the PO and optionally the propanal.

In some embodiments of the aforementioned aspects and embodiments, the base comprises alkali metal hydroxide and/or alkali earth metal hydroxide.

In some embodiments of the aforementioned aspects and embodiments, the bromination results in between about 20-95 w % yield of PBH.

In some embodiments of the aforementioned aspects and embodiments, reaction conditions for the bromination reaction comprise temperature of the reaction between 40-120° C.; concentration of the metal bromide with metal ion in the higher oxidation state entering the bromination to be between 0.5-3M; concentration of the metal bromide with metal ion in the lower oxidation state entering the bromination to be between 0.01-2M; or combinations thereof.

In some embodiments of the aforementioned aspects and embodiments, the method further comprises, before the bromination, contacting an anode with an anode electrolyte in an electrochemical cell wherein the anode electrolyte comprises metal bromide with metal ion in higher oxidation state, metal bromide with metal ion in lower oxidation state, and saltwater; contacting a cathode with a cathode electrolyte in the electrochemical cell; applying voltage to the anode and the cathode and oxidizing the metal bromide with the metal ion in the lower oxidation state to the higher oxidation state at the anode; and transferring the anode electrolyte from the electrochemical cell to the bromination reaction.

In some embodiments of the aforementioned aspects and embodiments, the method further comprises forming sodium hydroxide or potassium hydroxide in the cathode electrolyte and using the sodium hydroxide or the potassium hydroxide as the base to form the PO.

In some embodiments of the aforementioned aspects and embodiments, the one or more products from propylene further comprise hydrobromic acid (HBr).

In some embodiments of the aforementioned aspects and embodiments, the method further comprises forming sodium hydroxide in the cathode electrolyte and using the sodium hydroxide to neutralize the HBr.

In some embodiments of the aforementioned aspects and embodiments, the method further comprises after the bromination, oxybrominating the metal bromide with the metal ion in the lower oxidation state to the higher oxidation state in presence of oxygen and optionally HBr.

In some embodiments of the aforementioned aspects and embodiments, the method further comprises recirculating the metal bromide with the metal ion in the higher oxidation state back to the bromination reaction and/or back to the anode electrolyte of the electrochemical cell.

In some embodiments of the aforementioned aspects and embodiments, reaction conditions for the oxybromination reaction comprise temperature between about 50-100° C.; pressure between about 1-100 psig; oxygen partial pressure in feed to the oxybromination in a range between about 0.01-100 psia; or combinations thereof.

In some embodiments of the aforementioned aspects and embodiments, concentration of the metal bromide with the metal ion in the lower oxidation state entering the oxybromination reaction is between about 0.3-2M; concentration of the metal bromide with the metal ion in the lower oxidation state entering the bromination reaction is between about 0.01-2M; concentration of the metal bromide with the metal ion in the lower oxidation state entering the electrochemical reaction is between about 0.3-2.5M; or combinations thereof.

In some embodiments of the aforementioned aspects and embodiments, one or more of the oxidizing at the anode, the brominating, the hydrolyzing, the oxybrominating, and the epoxidizing reactions are carried out in the saltwater.

In some embodiments of the aforementioned aspects and embodiments, the saltwater is an alkali metal bromide selected from the group consisting of sodium bromide, potassium bromide, lithium bromide, and combinations thereof or alkali earth metal bromide selected from the group consisting of calcium bromide, strontium bromide, magnesium bromide, and combinations thereof.

In some embodiments of the aforementioned aspects and embodiments, the alkali metal bromide is sodium bromide or potassium bromide.

In some embodiments of the aforementioned aspects and embodiments, yield of the PO is more than 80 wt % and/or space time yield (STY) of the PO is more than 0.1 (mol/L/hr).

In some embodiments of the aforementioned aspects and embodiments, the metal bromide with the metal ion in the lower oxidation state is CuBr and the metal bromide with the metal ion in the higher oxidation state is CuBr₂.

In one aspect, there are provided systems, comprising:

a bromination reactor configured to receive an aqueous medium comprising metal bromide with metal ion in higher oxidation state, metal bromide with metal ion in lower oxidation state, and saltwater and brominate propylene with the metal bromide with the metal ion in the higher oxidation state to result in one or more products comprising PBH and DBP, and the metal bromide with the metal ion in the lower oxidation state;

an epoxide reactor operably connected to the bromination reactor and configured to receive the one or more products comprising PBH and DBP and epoxidize with a base to form PO and unreacted DBP; and

a hydrolysis reactor operably connected to the epoxide reactor and configured to receive the unreacted DBP from the epoxide reactor and hydrolyze under one or more reaction conditions to result in hydrolysis products comprising PBH and propanal.

In some embodiments of the aforementioned aspect and embodiments, the system further comprises an electrochemical cell operably connected to the bromination reactor, the hydrolysis reactor, and/or the epoxide reactor, comprising an anode in contact with an anode electrolyte wherein the anode electrolyte comprises metal bromide with metal ion in higher oxidation state, metal bromide with metal ion in lower oxidation state, and saltwater; a cathode in contact with a cathode electrolyte; and a voltage source configured to apply voltage to the anode and the cathode wherein the anode is configured to oxidize the metal bromide with the metal ion from the lower oxidation state to the higher oxidation state.

In some embodiments of the aforementioned aspect and embodiments, the system further comprises an oxybromination reactor operably connected to the electrochemical cell and/or the bromination reactor and configured to oxybrominate the metal bromide with the metal ion from the lower oxidation state to the higher oxidation state in presence of HBr and oxygen.

In some embodiments of the aforementioned aspect and embodiments, the electrochemical cell, the bromination reactor, the hydrolysis reactor, the epoxide reactor, and the oxybromination reactor are all configured to carry out the reactions in the saltwater.

In one aspect, there are provided methods comprising:

(i) contacting an anode with an anode electrolyte in an electrochemical cell wherein the anode electrolyte comprises metal bromide and saltwater; contacting a cathode with a cathode electrolyte in the electrochemical cell; applying voltage to the anode and the cathode and oxidizing the metal bromide with metal ion in a lower oxidation state to a higher oxidation state at the anode;

(ii) withdrawing the anode electrolyte from the electrochemical cell and brominating propylene with the anode electrolyte comprising the metal bromide with the metal ion in the higher oxidation state and the saltwater to result in one or more products comprising propylene bromohydrin (PBH) and the metal bromide with the metal ion in the lower oxidation state; or

withdrawing the anode electrolyte from the electrochemical cell and brominating ethylene with the anode electrolyte comprising the metal bromide with the metal ion in the higher oxidation state and the saltwater to result in one or more products comprising bromoethanol (BE) and the metal bromide with the metal ion in the lower oxidation state; and

(iii) epoxidizing the PBH or the BE with a base to form propylene oxide (PO) or ethylene oxide (EO), respectively.

In some embodiments of the aforementioned aspect, the method further comprises oxybrominating the metal bromide with the metal ion in the lower oxidation state to the higher oxidation state in presence of oxygen and optionally HBr.

In some embodiments of the aforementioned aspect and embodiments, the method further comprises recirculating the metal bromide with the metal ion in the higher oxidation state back to step (ii).

In some embodiments of the aforementioned aspect and embodiments, wherein reaction conditions for the oxybromination reaction comprise temperature between about 50-100° C.; pressure between about 1-100 psig; oxygen partial pressure in feed to the oxybromination in a range between about 0.01-100 psia; or combinations thereof.

In some embodiments of the aforementioned aspect and embodiments, the one or more products from propylene further comprise 1,2-dibromopropane (DBP) or the one or more products from ethylene further comprise 1,2-dibromoethane (DBE).

In some embodiments of the aforementioned aspect and embodiments, the bromination results in more than 20% yield of PBH or more than 20% yield of BE.

In some embodiments of the aforementioned aspect and embodiments, the reaction conditions for the bromination reaction comprise temperature of the reaction between 40-120° C.; concentration of the metal bromide with metal ion in the higher oxidation state entering the bromination to be between 0.8-3M; concentration of the metal bromide with metal ion in the lower oxidation state entering the bromination to be between 0.01-2M; or combinations thereof.

In some embodiments of the aforementioned aspect and embodiments, the method further comprises forming sodium hydroxide in the cathode electrolyte and using the sodium hydroxide as the base to form the propylene oxide or the ethylene oxide.

In some embodiments of the aforementioned aspect and embodiments, the one or more products from propylene or ethylene further comprise hydrobromic acid (HBr).

In some embodiments of the aforementioned aspect and embodiments, the method further comprises forming sodium hydroxide in the cathode electrolyte and using the sodium hydroxide to neutralize the HBr.

In some embodiments of the aforementioned aspect and embodiments, the oxidizing, the brominating and the oxybrominating steps are carried out in the saltwater.

In some embodiments of the aforementioned aspect and embodiments, the saltwater is an alkali metal bromide selected from the group consisting of sodium bromide, potassium bromide, and lithium bromide. In some embodiments of the aforementioned aspect and embodiments, the alkali metal bromide is sodium bromide.

In some embodiments of the aforementioned aspect and embodiments, the method further comprises separating the one or more products from the metal bromide and the saltwater.

In some embodiments of the aforementioned aspect and embodiments, the method further comprises separating the PBH or the BE from the metal bromide and the saltwater.

In some embodiments of the aforementioned aspect and embodiments, concentration of the metal bromide with the metal ion in the lower oxidation state entering the oxybromination reaction is between about 0.3-2M; concentration of the metal bromide with the metal ion in the lower oxidation state entering the bromination reaction is between about 0.01-2M; concentration of the metal bromide with the metal ion in the lower oxidation state entering the electrochemical reaction is between about 0.3-2.5M; or combinations thereof.

In some embodiments of the aforementioned aspect and embodiments, the method further comprises separating the metal bromide solution from the one or more products comprising PBH or the BE after the brominating step and delivering the metal bromide solution back to the electrochemical reaction and/or the oxybromination reaction.

In some embodiments of the aforementioned aspect and embodiments, yield of the PO or yield of the EO is more than 90 wt % and/or space time yield (STY) of the PO or STY of the EO is more than 0.1.

In some embodiments of the aforementioned aspect and embodiments, the metal bromide with the metal ion in the lower oxidation state is CuBr and the metal bromide with the metal ion in the higher oxidation state is CuBr₂.

In some embodiments of the aforementioned aspects and embodiments, the method further comprises separating the sodium bromide from the epoxidation step and/or the neutralization step and delivering the sodium bromide back to the electrochemical reaction to minimize or eliminate waste water production. In some embodiments, the sodium bromide from the epoxidation step may be re-circulated to a process producing HBr from Br₂. The HBr can then be sent to the oxybromination step, such as in FIGS. 3A and 3B (re-circulation not shown in FIGS. 3A and 3B).

In one aspect, there is provided a system, comprising:

an electrochemical cell comprising an anode in contact with an anode electrolyte wherein the anode electrolyte comprises metal bromide and saltwater; a cathode in contact with a cathode electrolyte; and a voltage source configured to apply voltage to the anode and the cathode wherein the anode is configured to oxidize the metal bromide with the metal ion from a lower oxidation state to a higher oxidation state; and/or an oxybromination reactor operably connected to the electrochemical cell and/or bromination reactor and configured to oxybrominate the metal bromide with the metal ion from the lower oxidation state to the higher oxidation state in presence of HBr and oxygen;

a bromination reactor operably connected to the electrochemical cell and/or the oxybromination reactor wherein the bromination reactor is configured to receive the metal bromide with the metal ion in the higher oxidation state from the electrochemical cell and/or configured to receive the metal bromide solution with the metal ion in the higher oxidation state from the oxybromination reactor and brominate propylene or ethylene with the metal bromide with the metal ion in the higher oxidation state to result in one or more products comprising PBH or one or more products comprising BE, respectively, and the metal bromide solution with the metal ion in the lower oxidation state; and

an epoxide reactor operably connected to the bromination reactor and/or the oxybromination reactor and configured to epoxidize the PBH or the BE with a base to form PO or EO, respectively.

In some embodiments of the aforementioned aspect, the electrochemical cell, the bromination reactor and the oxybromination reactor are all configured to carry out the reactions in the alkali metal bromide in the water. In some embodiments of the aforementions aspect and embodiments, the epoxide reactor is operably connected to the electrochemical cell wherein the electrochemical cell is configured to receive some or all of the saltwater, e.g. alkali metal bromide from the epoxide reactor.

### BRIEF DESCRIPTION OF THE DRAWINGS

The novel features of the invention are set forth with particularity in the appended claims. A better understanding of the features and advantages of the present invention may be obtained by reference to the following detailed description that sets forth illustrative embodiments, in which the principles of the invention are utilized, and the accompanying drawings of which:

FIG. 1A is an illustration of some embodiments related to the bromination reaction, the epoxidation reaction, and the hydrolysis reaction using propylene.

FIG. 1B is an illustration of some embodiments related to the bromination reaction, and the epoxidation reaction, and the hydrolysis reaction using ethylene.

FIG. 2A is an illustration of some embodiments related to the hydrolysis reaction of DBP.

FIG. 2B is an illustration of some embodiments related to the hydrolysis reaction of DBE.

FIG. 3A is an illustration of some embodiments related to the electrochemical reaction, the bromination reaction, the neutralization reaction, and the epoxidation reaction using propylene.

FIG. 3B is an illustration of some embodiments related to the electrochemical reaction, the bromination reaction, the neutralization reaction, and the epoxidation reaction using ethylene.

FIG. 4A is an illustration of some embodiments related to the electrochemical reaction, the oxybromination reaction, the bromination reaction, and the epoxidation reaction using propylene.

FIG. 4B is an illustration of some embodiments related to the electrochemical reaction, the oxybromination reaction, the bromination reaction, and the epoxidation reaction using ethylene.

FIG. 5A is an illustration of some embodiments related to the oxybromination reaction, the bromination reaction, the hydrolysis reaction, and the epoxidation reaction using propylene.

FIG. 5B is an illustration of some embodiments related to the oxybromination reaction, the bromination reaction, the hydrolysis reaction, and the epoxidation reaction using ethylene.

FIG. 6A is an illustration of some embodiments related to the electrochemical reaction, oxidation reaction, the bromination reaction, the oxybromination reaction, and the epoxidation reaction using propylene.

FIG. 6B is an illustration of some embodiments related to the electrochemical reaction, the oxidation reaction, the bromination reaction, the oxybromination reaction, and the epoxidation reaction using ethylene.

FIG. 7 is an illustration of some embodiments of an electrochemical cell.

FIG. 8 is an illustration of some embodiments of an electrochemical cell.

### DETAILED DESCRIPTION

Disclosed herein are systems and methods that relate to various combinations of an electrochemical, bromination, oxybromination, hydrolysis, and epoxidation methods and systems, to form propylene oxide (PO) or ethylene oxide (EO). These combined methods and systems provide an efficient, low cost, and low energy consuming systems that use metal bromide redox shuttles to form propylene bromohydrin (PBH) (exclusively or with formation of 1,2-dibromopropane or dibromopropane (DBP) and/or propanal and other products described herein) from propylene and its subsequent epoxidation to PO; or to form bromoethanol (BE) (exclusively or with formation of 1,2-dibromoethane or dibromoethane (DBE) and/or other products described herein) from ethylene and its subsequent epoxidation to EO.

“Bromoethanol” or “BE” as used interchangeably herein is also known as 2-bromoethanol, ethylbromohydrin (EBH), etc.

“1,2-dibromoethane” or “dibromoethane” or “DBE” as used interchangeably herein is also known as ethylene dibromide or EDB.

“1,2-dibromopropane” or “dibromopropane” or “DBP” as used interchangeably herein is also known as propylene dibromide or PDB.

“Propylene bromohydrin” or “PBH” as used interchangeably herein is also known as bromopropyl alcohol and may be present in one or more of its isomeric forms such as, 1-hydroxy-2-bromopropane, 1-bromo-2-hydroxypropane, or combination thereof.

“Propionaldehyde” or “propanal” as used herein is an organic compound with formula CH₃CH₂CHO.

The structure of the aforementioned compounds has been shown in the figures.

The systems and methods provided herein are configured with saltwater, e.g., an alkali metal ion or alkaline earth metal ion solution, e.g. potassium bromide solution or sodium bromide solution or lithium bromide solution or a magnesium bromide solution or calcium bromide solution or strontium bromide, to optionally produce an equivalent alkaline solution, e.g., potassium hydroxide or sodium hydroxide or lithium hydroxide or magnesium hydroxide or calcium hydroxide or strontium hydroxide in the cathode electrolyte (or other reactions at the cathode described herein). In some embodiments, the saltwater is ammonium bromide solution producing a corresponding ammonium hydroxide at the cathode (or other reactions at the cathode described herein). This saltwater can be used as an anode electrolyte, cathode electrolyte, and/or brine in the middle compartment of the electrochemical cell. Accordingly, to the extent that such equivalents are based on or suggested by the present system and method, these equivalents are within the scope of the application.

Before the present invention is described in greater detail, it is to be understood that this invention is not limited to particular embodiments described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges that are presented herein with numerical values may be construed as “about” numerical. The “about” is to provide literal support for the exact number that it precedes, as well as a number that is near to or approximately the number that the term precedes. In determining whether a number is near to or approximately a specifically recited number, the near or approximating unrequited number may be a number, which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a,” “an,” and “the” include plural references unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods and Systems

There are provided methods and systems that relate to various combinations of an electrochemical, bromination, hydrolysis, oxybromination and epoxidation methods and systems, to form PO or EO.

Typically, bromide systems are less attractive compared to chloride systems because bromide salts are more expensive than the chloride salts and the waste streams from the bromide systems are difficult to handle and process. However, Applicants surprisingly found that the use of bromide (such as metal bromide and alkali metal or alkali earth metal bromide) in various combinations and reaction conditions of the electrochemical, the bromination, the oxybromination, the hydrolysis, and the epoxidation methods and systems as described herein, enhances the yield and selectivity of PBH and PO or BE and EO and provides several economic advantages as listed below. For example, this enhancement in the yield and selectivity of the PBH obtained from the propylene in the bromination reaction/reactor as well as the PO obtained from the PBH in the epoxidation reaction/reactor was dramatically higher than that obtained via chlorination process, ie. where propylene chlorohydrin (PCH) was formed and PO was then obtained from PCH.

Applicants observed that substituting cupric chloride (CuCl₂) with cupric bromide (CuBr₂) led to a dramatic increase in the rate of the propylene conversion to the desired products as measured by the space time yield (STY). For example, Applicants found that, in some embodiments, the amount of the dibromopropane (DBP) formed from the propylene in the bromination reaction/reactor was considerably lower or negligible and the amount of PBH was considerably higher compared to the metal chloride methods and systems where higher amount of dichloropropane was formed. Therefore, in some embodiments, the fraction of the PBH to the total of PBH and DBP (PBH/PBH+DBP) is higher than the fraction of the PCH to the total of PCH and DCP (PCH/PCH+DCP).

The dichloropropane is converted in a second step to PCH, typically in a second reactor with a catalytic system. However, Applicants observed that substituting metal bromide, e.g. CuBr₂ for CuCl₂ dramatically increased the amount of propylene converted directly to the PBH. It was further observed that, in some embodiments, whatever small amount of the DBP that was formed after reaction of the propylene, the conversion and selectivity of the reaction transforming the DBP to the PBH was higher than the conversion and selectivity of the reaction transforming dichloropropane to PCH. For example, in some embodiments, the DBP to the PBH yielded a selectivity of approximately 80% or 90% or more. Furthermore, in addition to the improved selectivity, it was found that the DBP to the PBH formation reaction could be performed using the anode electrolyte as the catalytic solution rather than requiring a second catalyst system, which significantly reduced process complexity especially with regard to the recovery and reuse of the resulting acid.

Applicants also surprisingly found that, in some embodiments, the hydrolysis of the DBP to the PBH also resulted in the formation of certain commercially valuable products, such as, but not limited to, propanal, bromopropanal, dibromopropanal, or combination thereof which can be isolated and sold for commercial purposes. Propanal is a common reagent, being a building block to many compounds. For example, its used as a precursor to trimethylolethane (CH₃C(CH₂OH)₃) through a condensation reaction with formaldehyde. This triol is an important intermediate in the production of alkyd resins. The methods and systems provided herein result in the formation of one or more products, including, but not limited to, PBH, DBP, and propanal, some of which can be used further to form PO and/or separated to be sold as is.

Other side products that can be formed during the hydrolysis of the DBP to the PBH include, but not limited to, acetone, bromoacetone, dibromoacetone, bromopropenes, or combinations thereof. All of these products have been described further herein.

It was also observed that the use of bromination methods and systems not only reduced the operating temperature of the electrochemical cell/reaction as well as the bromination reactor/reaction but also the amount of metal bromide needed to achieve same or higher STY compared to the chlorination methods and systems. It was observed that due to higher solubility of the metal bromide and bromide salts in water at room temperature, the electrochemical methods and systems may be run at a lower temperature compared to the chlorination methods and systems. Similarly, the bromination reaction may also be run at a lower temperature as the reaction with the bromide system is much faster than the chloride system. For example, in the CuBr₂ system, an STY of 0.5 or 1 can be achieved at 100° C. or higher and at CuBr₂ concentrations as low as 1 mole/kg. The lower temperature of the bromination system, the lower amount of the metal bromide, and/or the higher STY result in several economic advantages including, but not limited to, minimized reactor size, reduced heating/cooling costs, and other process economic advantages. The lower concentration of the metal bromides in the bromination methods and systems also results in better solubility and workability.

In addition to the several advantages listed above related to the bromination chemistry, the bromide methods and systems also improve the propylene oxide purification steps. Like most industrial chemicals, propylene oxide may be purified primarily by distillation, which may rely on differences in boiling points to separate compounds. One of the challenges in the metal chloride process may be the removal of chlorinated side products from the PO because the boiling points of some chlorinated side products, such as but not limited to, isopropyl chloride and 1-chloropropene, may be within a few degrees of PO. The proximity of the boiling points can render distillation ineffective. Therefore, these side products need to be minimized or removed prior to the formation of the PO in epoxidation. However, the bromination methods and systems can avoid this extraneous step because the closest brominated C3 has a boiling point that may be 13° C. away from the PO. The other products such as DBP, propanal, and bromopropanals formed in the methods and systems provided herein, can be easily separated from PO via distillation.

Another significant advantage of the bromination methods and systems is that the brominated compounds, such as the DBP, have a significantly different liquid density than the aqueous solutions. This helps in process steps where liquid phases can be separated by gravity.

Finally, there is an additional advantage in using the bromide methods and systems in the electrochemical cell. Typically, the anion exchange membranes are manufactured with brominated functional groups that have to be exchanged to chloride groups for the chloride method/system. Such exchange becomes redundant in the bromide methods and systems improving the economics of the process even further.

In one aspect, there are provided methods that include:

brominating propylene with an aqueous medium comprising metal bromide with metal ion in higher oxidation state, metal bromide with metal ion in lower oxidation state, and saltwater to result in one or more products comprising DBP and PBH and reduction of the metal bromide with the metal ion in the higher oxidation state to the metal bromide with the metal ion in the lower oxidation state;

epoxidizing the one or more products comprising DBP and PBH with a base to form PO and unreacted DBP; and

hydrolyzing the unreacted DBP under one or more reaction conditions to result in hydrolysis products comprising PBH and propanal.

The “unreacted DBP” as used herein includes the DBP that remains unchanged after the reaction. For example, in the aforementioned aspect, the DBP that remains unchanged after the epoxidation reaction is unreacted DBP.

The “base” used herein in the epoxidation reaction/reactor may be any known base in the art. Examples include, without limitation, alkali metal hydroxides, alkaline earth metal hydroxides, and the like. In some embodiments, the sodium hydroxide in the cathode electrolyte is used as the base optionally supplemented with other bases as listed herein. In some embodiments, metal hydroxybromide may also be used as a base. The metal hydroxybromides have been described herein.

In some embodiments of the aforementioned aspect, the one or more products comprising DBP and PBH are separated from the aqueous medium comprising metal bromide with metal ion in higher oxidation state, metal bromide with metal ion in lower oxidation state, and saltwater, before subjecting the one or more products comprising DBP and PBH to the epoxidation. Various methods of separation such as extraction, have been described herein. Similar methods of separation such as distillation can be employed to separate the PO and the unreacted DBP after the epoxidation reaction. In some embodiments of the aforementioned aspect, the hydrolysis products comprising PBH and propanal are sent back to the epoxidation reaction where the PBH reacts with a base to form PO (leaving propanal as unreacted propanal).

Similar to the aforementioned aspect, there are provided methods comprising

brominating ethylene with an aqueous medium comprising metal bromide with metal ion in higher oxidation state, metal bromide with metal ion in lower oxidation state, and saltwater to result in one or more products comprising DBE and BE and reduction of the metal bromide with the metal ion in the higher oxidation state to the metal bromide with the metal ion in the lower oxidation state;

epoxidizing the one or more products comprising DBE and BE with a base to form EO and unreacted DBE; and

hydrolyzing the unreacted DBE under one or more reaction conditions to result in hydrolysis products comprising BE and one or more of acetaldehyde, bromoacetaldehyde, dibromoacetaldehyde, tribromoacetaldehyde, or combinations thereof.

The “unreacted DBE” as used herein includes the DBE that remains unchanged after the reaction. For example, in the aforementioned aspect, the DBE that remains unchanged after the epoxidation reaction is unreacted DBE.

In some embodiments of the aforementioned aspect, the one or more products comprising DBE and BE are separated from the aqueous medium comprising metal bromide with metal ion in higher oxidation state, metal bromide with metal ion in lower oxidation state, and saltwater, before subjecting the one or more products comprising DBE and BE to the epoxidation. Various methods of separation such as extraction, have been described herein. Similar methods of separation such as distillation can be employed to separate the EO and the unreacted DBE after the epoxidation reaction. In some embodiments of the aforementioned aspect, the hydrolysis products comprising BE and one or more of acetaldehyde, bromoacetaldehyde, dibromoacetaldehyde, tribromoacetaldehyde, or combinations thereof are sent back to the epoxidation reaction where the BE reacts with a base to form EO (separating one or more of acetaldehyde, bromoacetaldehyde, dibromoacetaldehyde, tribromoacetaldehyde, or combinations thereof as unreacted).

The methods and systems provided herein are sometimes closed-loop processes, therefore, the order of one or more steps provided herein may be alternated or rearranged and the steps are not necessarily arranged in a serial fashion.

Accordingly, in an aspect, there are provided methods that include:

brominating propylene with an aqueous medium comprising metal bromide with metal ion in higher oxidation state, metal bromide with metal ion in lower oxidation state, and saltwater to result in one or more products comprising DBP and reduction of the metal bromide with the metal ion in the higher oxidation state to the metal bromide with the metal ion in the lower oxidation state;

subjecting the one or more products comprising DBP to hydrolysis under one or more reaction conditions to result in hydrolysis products comprising PBH and propanal; and

epoxidizing the hydrolysis products comprising PBH and propanal with a base to form PO.

In some embodiments of the aforementioned aspect, the one or more products in bromination reaction further comprise PBH.

In some embodiments of the aforementioned aspect, the one or more products comprising DBP are separated from the aqueous medium comprising metal bromide with metal ion in higher oxidation state, metal bromide with metal ion in lower oxidation state, and saltwater, before subjecting the one or more products comprising DBP to hydrolysis. Various methods of separation such as extraction, have been described herein. In some embodiments of the aforementioned aspect, the method comprises without separating, subjecting the aqueous medium comprising metal bromide with metal ion in higher oxidation state, metal bromide with metal ion in lower oxidation state, and saltwater, and the one or more products comprising DBP, to the hydrolysis reaction.

In some embodiments, in the aforementioned aspect, some or all of the propanal may remain unchanged after the epoxidation reaction such that epoxidizing the hydrolysis products comprising PBH and propanal with a base forms PO and unreacted propanal. Applicants observed that the ability to epoxidize PBH to PO in the presence of other compounds such as DBP, propanal, and bromopropanals, reduces the number of steps in the process providing significant economic advantage and reduced loss of products. The epoxidation reaction also serves as a separation step to separate out the PO from unreacted DBP and unreacted propanal which after separation provide products with significant commercial value.

The “unreacted propanal” as used herein includes the propanal that remains unchanged after the reaction. In some embodiments of the aforementioned aspect, various methods of separation such as distillation are employed to separate the PO and the unreacted propanal after the epoxidation reaction. The separated and purified PO and propanal can be sold commercially.

In one aspect, there are provided methods that include:

brominating ethylene with an aqueous medium comprising metal bromide with metal ion in higher oxidation state, metal bromide with metal ion in lower oxidation state, and saltwater to result in one or more products comprising DBE and reduction of the metal bromide with the metal ion in the higher oxidation state to the metal bromide with the metal ion in the lower oxidation state;

subjecting the one or more products comprising DBE to hydrolysis under one or more reaction conditions to result in hydrolysis products comprising BE and one or more of acetaldehyde, bromoacetaldehyde, dibromoacetaldehyde, tribromoacetaldehyde, or combinations thereof; and

epoxidizing the hydrolysis products comprising BE and one or more of acetaldehyde, bromoacetaldehyde, dibromoacetaldehyde, tribromoacetaldehyde, or combinations thereof with a base to form EO.

In some embodiments of the aforementioned aspect, the one or more products further comprise BE.

In some embodiments of the aforementioned aspect, the one or more products comprising DBE are separated from the aqueous medium comprising metal bromide with metal ion in higher oxidation state, metal bromide with metal ion in lower oxidation state, and saltwater, before subjecting the one or more products comprising DBE to hydrolysis. Various methods of separation such as extraction, have been described herein. In some embodiments of the aforementioned aspect, the method comprises without separating, subjecting the aqueous medium comprising metal bromide with metal ion in higher oxidation state, metal bromide with metal ion in lower oxidation state, and saltwater, and the one or more products comprising DBE, to the hydrolysis reaction.

In some embodiments, in the aforementioned aspect, some or all of the one or more of acetaldehyde, bromoacetaldehyde, dibromoacetaldehyde, tribromoacetaldehyde, or combinations thereof may remain unchanged after the epoxidation reaction such that epoxidizing the hydrolysis products comprising BE and the one or more of acetaldehyde, bromoacetaldehyde, dibromoacetaldehyde, tribromoacetaldehyde, or combinations thereof with a base forms EO and unreacted one or more of acetaldehyde, bromoacetaldehyde, dibromoacetaldehyde, tribromoacetaldehyde, or combinations thereof. The “unreacted” as used herein includes a compound that remains unchanged after a reaction. In some embodiments of the aforementioned aspect, various methods of separation such as distillation are employed to separate the EO and the unreacted one or more of acetaldehyde, bromoacetaldehyde, dibromoacetaldehyde, tribromoacetaldehyde, or combinations thereof, after the epoxidation reaction. The separated and purified EO and one or more of acetaldehyde, bromoacetaldehyde, dibromoacetaldehyde, tribromoacetaldehyde, or combinations thereof can be sold commercially.

“Saltwater” as used herein includes water comprising alkali metal ions such as, alkali metal bromides e.g. sodium bromide, potassium bromide, lithium bromide, etc. and/or water comprising alkali earth metal ions such as, alkali earth metal bromides e.g. magnesium bromide, calcium bromide, strontium bromide, etc. It can also be a combination of the alkali metal bromide and alkali earth metal bromide.

In some embodiments of the aforementioned aspects using propylene, the hydrolysis reaction further comprises bromopropanal, dibromopropanal, or combinations thereof. In some embodiments of the aforementioned aspects using propylene, the hydrolysis reaction further comprises acetone, bromoacetone, dibromoacetone, or combinations thereof. In some embodiments of the aforementioned aspects using propylene, the hydrolysis reaction further comprises unreacted DBP. In some embodiments of the aforementioned aspects using propylene, the hydrolysis reaction further comprises bromopropenes.

In some embodiments of the aforementioned aspects using ethylene, the hydrolysis reaction further comprises unreacted DBE.

In some embodiments of the aforementioned aspects using propylene, the method comprising forming the PO and the one or more of the unreacted DBP, the unreacted PBH, the unreacted propanal (or bromo propanals as listed above), or combinations thereof, after the epoxidation reaction depending on the operable connection of the epoxidation reaction with the bromination and/or the hydrolysis reaction. The unreacted DBP can be subjected to the hydrolysis again to form PBH which can then be sent to epoxidation reaction. The “unreacted PBH” as used herein includes the PBH that remains unchanged after the reaction.

Production and ratio of the aforementioned products can be controlled using specific organic:aqueous ratios as explained further herein below.

The bromination reaction, the epoxidation reaction, and the hydrolysis reaction, as described in the aforementioned aspects and embodiments, are as follows.

Bromination to Form PBH and Optionally DBP from Propylene or to Form BE and Optionally DBE from Ethylene

The “bromination” or its grammatical equivalent, as used herein, includes a reaction of the propylene or the ethylene with the metal bromide with the metal ion in the higher oxidation state and saltwater to form one or more products. The “one or more products” used herein includes organic and optionally inorganic products formed during the bromination reaction. The organic one or more products comprise PBH (including enantiomers thereof) and other products formed during the reaction with the propylene or the organic one or more products comprise BE and optionally other products formed during the reaction with ethylene. In some embodiments of the above noted aspect, the one or more products from propylene further comprise dibromopropane (DBP) or the one or more products from ethylene further comprise dibromoethane (DBE).

In some embodiments of the above noted aspect and embodiments, the bromination results in more than 20%; or more than 30%; or more than 40%; or more than 50%; or more than 60%; or more than 70%; or more than 80%; or more than 90% yield of PBH or BE. In some embodiments, the remaining % is of DBP and/or other products or the remaining % is of DBE and/or other side products. In some embodiments, no DBP and/or DBE may be formed. The other products include, without limitation, other brominated derivatives from propylene or other brominated derivatives from ethylene.

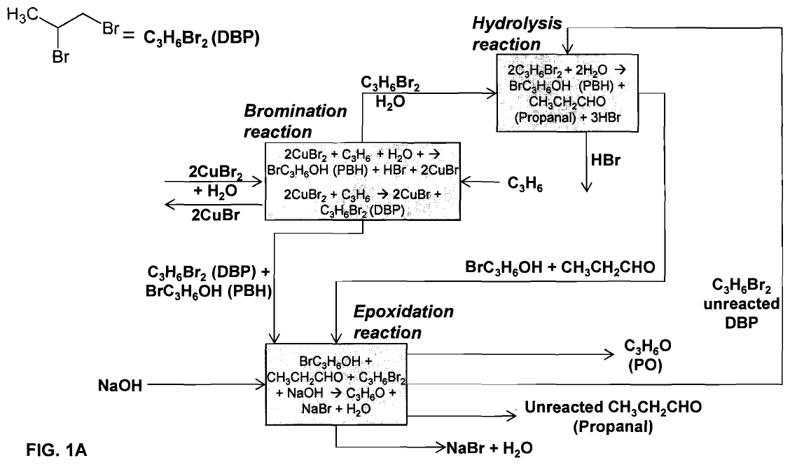

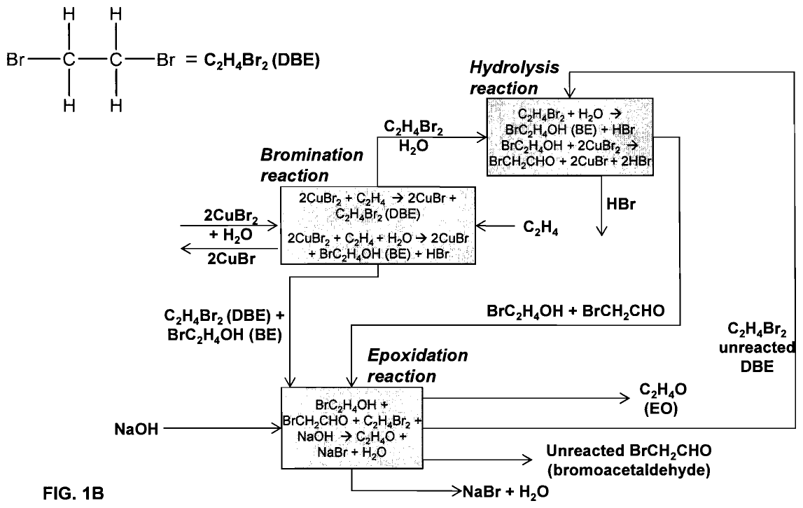

The bromination of propylene to form one or more products comprising PBH and/or DBP is illustrated in FIGS. 1A, 3A, 4A, 5A, and 6A and the bromination of ethylene to form one or more products comprising BE and/or DBE is illustrated in FIGS. 1B, 3B, 4B, 5B, and 6B. FIG. 1A illustrates formation of both DBP and PBH in the bromination reaction of propylene. It is to be understood that the bromination reaction can form either DBP or PBH or combination thereof depending on the reaction conditions, as described herein. It is also to be understood that either PBH or DBP can be formed as major product and the other as a minor product depending on the reaction conditions. Similarly, FIG. 1B illustrates formation of both DBE and BE in the bromination reaction of ethylene. It is to be understood that the bromination reaction can form either DBE or BE or combination thereof depending on the reaction conditions, as described herein. It is also to be understood that either BE or DBE can be formed as major product and the other as a minor product depending on the reaction conditions.

As shown in FIGS. 1A, 3A, 4A, 5A, and 6A, an aqueous medium comprising metal bromide with metal ion in higher oxidation state (illustrated as, e.g. CuBr₂), metal bromide with metal ion in lower oxidation state (illustrated as, e.g. CuBr), and saltwater (illustrated as, e.g. NaBr) is used to brominate propylene to form one or more products comprising DBP and/or PBH. Similarly, as shown in FIGS. 1B, 3B, 4B, 5B, and 6B, an aqueous medium comprising metal bromide with metal ion in higher oxidation state (illustrated as, e.g. CuBr₂), metal bromide with metal ion in lower oxidation state (illustrated as, e.g. CuBr), and saltwater (illustrated as, e.g. NaBr) is used to brominate ethylene to form one or more products comprising DBE and/or BE. In the bromination reaction, the metal bromide with the metal ion in the higher oxidation state oxidizes the hydrocarbon, such as ethylene or propylene and in-turn reduces to the metal bromide with the metal ion in the lower oxidation state.

It is to be understood that each reaction presented herein has a mixture of both metal bromide with metal ion in higher oxidation state (illustrated as e.g. CuBr₂) and metal bromide with metal ion in lower oxidation state (illustrated as e.g. CuBr), however, only the metal brmide involved in the reaction is shown in the figures. For example, FIGS. 1A, 3A, 4A, 5A, and 6A, illustrates CuBr₂ entering the bromination reaction and converting to CuBr, however, since the process is a closed loop process, the aqueous medium comprising CuBr₂ also has CuBr. The ratios of CuBr and CuBr₂ varies throughout the process depending on the oxidation or reduction reaction of the metal bromide.

The aqueous medium comprising metal bromide with metal ion in higher oxidation state (illustrated as, e.g. CuBr₂), metal bromide with metal ion in lower oxidation state (illustrated as, e.g. CuBr), and saltwater (illustrated as, e.g. NaBr) that is used to brominate propylene or ethylene, can be obtained from an anode electrolyte of an electrochemical reaction and/or solution from an oxybromination reaction and/or solution from a bromine oxidation reaction. All of these reactions have been described in detail herein.

As described earlier, in the bromide methods and systems provided herein, the amount of DBP may be negligible or in lower amounts compared to the DCP obtained in the chloride methods and systems. For example, Applicants observed that substituting metal bromide, e.g. CuBr₂ for CuCl₂ dramatically increased the amount of propylene converted directly to the propylene bromohydrin. It was further observed that whatever amount of DBP that was formed after reaction of the propylene, the conversion and selectivity of the reaction transforming the DBP to the PBH was higher than the conversion and selectivity of the reaction transforming the DCP to the PCH.

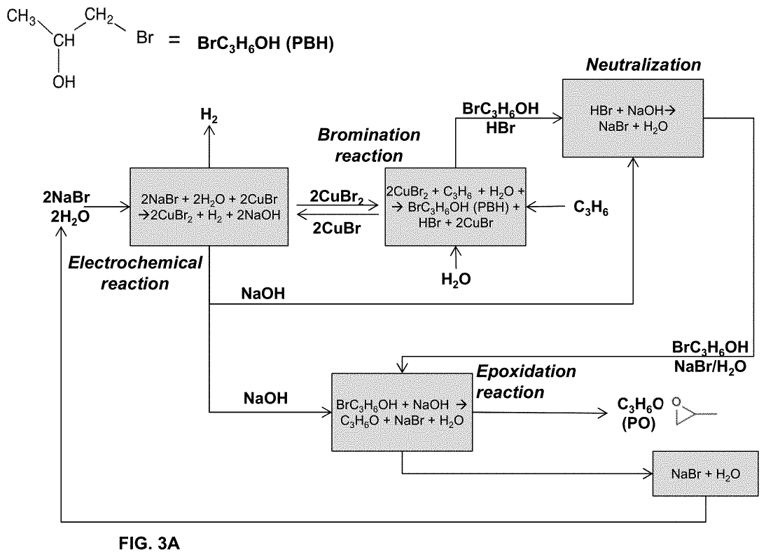

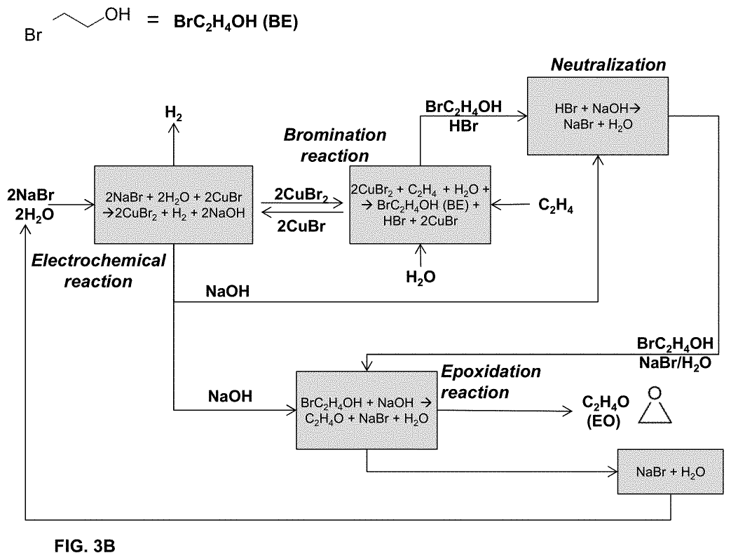

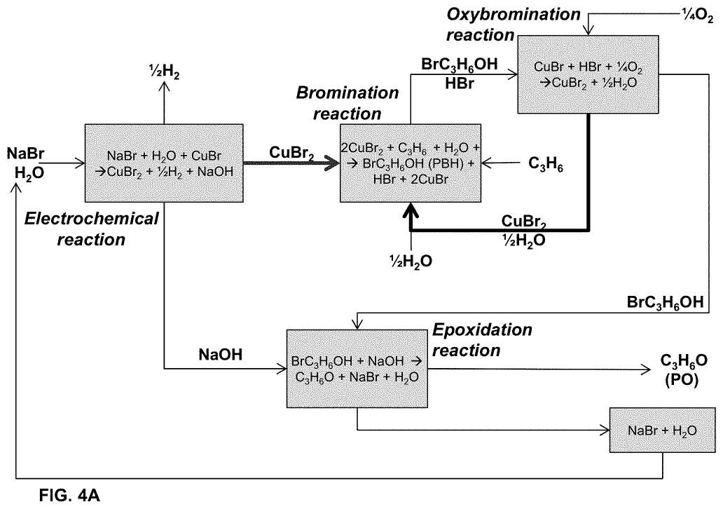

The PBH or BE may be separated from other products using separation techniques described herein. Other organic side products formed from propylene include without limitation, acetone. Example of inorganic products includes, without limitation, HBr. The HBr may be formed in the bromination reaction and may be present in the saltwater along with metal bromides. In some embodiments, the PBH or BE and other organic side products may be separated from the aqueous medium (saltwater containing metal bromides and HBr) and the HBr solution may be neutralized with NaOH (the NaOH may be formed in the electrochemical reaction described herein). The neutralization reaction has been illustrated in FIGS. 3A and 3B. As described earlier, the advantage of the bromination methods and systems is that the brominated compounds, such as the DBP, have a significantly different liquid density than the aqueous solutions. This helps in process steps where liquid phases can be separated by gravity.

In the bromination reactor, the propylene or ethylene may be supplied under pressure in the gas phase, or as a liquid in the case of propylene, and the metal bromide, for example only, copper(II) bromide (also containing copper(I) bromide) is supplied in an aqueous solution that may be originating from the outlet of the anode chamber of the electrochemical cell and/or originating from the outlet of the oxybromination reactor and/or originating from the outlet of the bromine oxidation reactor (described further herein). The reaction may occur in the liquid phase where the dissolved propylene or ethylene reacts with the copper(II) bromide. The reaction may be carried out at pressures between about 10-530 psig; or between about 10-500 psig; or between about 10-200 psig; or between about 10-100 psig; or between about 200-300 psig; or between about 10-50 psig to improve propylene or ethylene solubility in the aqueous phase. The bromide method and system provided herein allows the bromination reactor to be operated at significantly lower pressure which, in turn, reduces the pumping costs associated with pressurizing anolyte from the electrochemical cell and/or the oxybromination reactor up to reaction pressure. After the reaction, the metal ion in the higher oxidation state is reduced to the metal ion in the lower oxidation state. In some embodiments, the metal ion aqueous solution is separated from the one or more products (organics) in a separator before the metal ion solution is sent to the anode electrolyte of the electrochemical system and/or to the oxybromination reactor. The separated one or more products (organics) may be sent to the epoxidation reaction/reactor for the formation of the PO or sent to the hydrolysis reaction/reactor for the hydrolysis of the DBP to the PBH. In some embodiments, the metal ion aqueous solution is not separated from the one or more products (organics) and the aqueous medium comprising the metal bromide in the lower oxidation state, the metal bromide in the higher oxidation state, the saltwater and the one or more products are all sent to the hydrolysis reaction/reactor for the hydrolysis of the DBP to the PBH.

It is to be understood that the metal bromide solution going into the anode electrolyte and the metal bromide solution coming out of the anode electrolyte contains a mix of the metal bromide in the lower oxidation state and the higher oxidation state except that the metal bromide solution coming out of the anode chamber has higher amount of metal bromide in the higher oxidation state than the metal bromide solution going into the anode electrolyte.

As described earlier, the use of the bromination methods and systems as provided herein reduced the operating temperature of the system needed to achieve same or higher STY compared to the chlorination methods and systems. Applicants unexpectedly observed that the bromide system has a higher reaction rate compared to the chloride system, which may allow a lower temperature to be used without sacrificing reactor rate/size. Other unexpected advantages include, but not limited to, less decomposition of reactants and/or products, better process integration (no or smaller heat exchangers), cheaper materials of construction, etc. In some embodiments of the foregoing embodiments, the one or more reaction conditions for the bromination mixture or the reaction mixture in the bromination reactor are selected from temperature of between about 30-200° C.; or between about 30-180° C.; or between about 30-160° C.; or between about 30-140° C.; or between about 30-120° C.; or between about 30-100° C.; or between about 30-80° C.; or between about 30-70° C.; or between about 30-60° C.; or between about 30-50° C.; or between about 30-40° C.; or between about 40-200° C.; or between about 40-180° C.; or between about 40-160° C.; or between about 40-140° C.; or between about 40-120° C.; or between about 40-100° C.; or between about 40-80° C.; or between about 40-70° C.; or between about 40-60° C.; or between about 40-50° C.; or between about 50-200° C.; or between about 50-180° C.; or between about 50-160° C.; or between about 50-140° C.; or between about 50-120° C.; or between about 50-100° C.; or between about 50-80° C.; or between about 50-70° C.; or between about 50-60° C.; or between about 70-200° C.; or between about 70-180° C.; or between about 70-160° C.; or between about 70-140° C.; or between about 70-120° C.; or between about 70-100° C.; or between about 70-80° C.; or between about 80-180° C.; or between about 80-160° C.; or between about 80-140° C.; or between about 80-120° C.; or between about 80-100° C.; or between about 80-90° C.; or between about 90-180° C.; or between about 90-160° C.; or between about 90-140° C.; or between about 90-120° C.; or between about 90-100° C.; or between about 75-100° C.; or between about 75-110° C.; or between about 80-110° C.; or between about 135-180° C. It was observed that the operating temperature of the bromination reaction/system was lower than that of the clorination method/system thereby minimizing heating and cooling costs and other process economic advantages, as described earlier.

In some embodiments of the foregoing embodiments, the one or more reaction conditions for the bromination mixture or the reaction mixture in the bromination reactor are selected from incubation time of between about 1 sec-3 hour.

As described earlier, the use of bromination methods and systems not only reduced the operating temperature of the system but also the amount of metal bromide needed to achieve same or higher STY compared to the chlorination methods and systems. In some embodiments of the foregoing embodiments, the one or more reaction conditions for the bromination mixture or the reaction mixture in the bromination reactor are selected from concentration of the metal bromide in the higher oxidation state at more than 0.5M or between 0.5-3M. In some embodiments, the concentration of the metal bromide in the higher oxidation state is more than 0.5M; or more than 0.6M; or more than 0.7M; or more than 0.8M; or between 0.5-3M; or between 0.6-3M; or between 0.7-3M; or between 0.8-3M; or between 0.9-3M; or between 1-3M; or between 1.5-3M; or between 2-3M; or between 2.5-3M; or between 0.5-2.5M; or between 0.8-2.5M; or between 1-2.5M; or between 1.5-2.5M; or between 2-2.5M; or between 0.5-2M; or between 0.8-2M; or between 1-2M; or between 1.5-2M; or between 0.5-1.5M; or between 0.8-1.5M; or between 1-1.5M; or between 0.5-1M; or between 0.8-1M.

In some embodiments of the foregoing embodiments, the one or more reaction conditions for the bromination mixture or the reaction mixture in the bromination reactor are selected from concentration of the metal bromide in the lower oxidation state at more than 0.01M; or more than 0.05M; or between 0.01-2M; or between 0.01-1.8M; or between 0.01-1.5M; or between 0.01-1.2M; or between 0.01-1M; or between 0.01-0.8M; or between 0.01-0.6M; or between 0.01-0.5M; or between 0.01-0.4M; or between 0.01-0.1M; or between 0.01-0.05M; or between 0.05-2M; or between 0.05-1.8M; or between 0.05-1.5M; or between 0.05-1.2M; or between 0.05-1M; or between 0.05-0.8M; or between 0.05-0.6M; or between 0.05-0.5M; or between 0.05-0.4M; or between 0.05-0.1M; or between 0.1-2M; or between 0.1-1.8M; or between 0.1-1.5M; or between 0.1-1.2M; or between 0.1-1M; or between 0.1-0.8M; or between 0.1-0.6M; or between 0.1-0.5M; or between 0.1-0.4M; or between 0.5-2M; or between 0.5-1.8M; or between 0.5-1.5M; or between 0.5-1.2M; or between 0.5-1M; or between 0.5-0.8M; or between 0.5-0.6M; or between 1-2M; or between 1-1.8M; or between 1-1.5M; or between 1-1.2M; or between 1.5-2M.

It is to be understood that any combination of the aforementioned concentrations for the metal bromide in the lower oxidation state and the metal bromide in the higher oxidation state can be combined to achieve high yield and selectivity. For example only, in some embodiments of the foregoing embodiments, the one or more reaction conditions for the bromination mixture or the reaction mixture in the bromination reactor are selected from concentration of the metal bromide in the lower oxidation state of between about 0.01-2M or between about 0.01-1.5M or between about 0.01-1M and the concentration of the metal bromide in the higher oxidation state of between about 0.5-3M or between about 0.8-3M or between about 0.5-2M.

In some embodiments of the foregoing aspect and embodiments, the one or more reaction conditions for the bromination reaction comprise temperature between about 40-100° C., pressure between about 1-100 psig, or combination thereof. In some embodiments of the foregoing aspect and embodiments, reaction conditions for the bromination reaction comprise temperature of the reaction between 40-120° C.; concentration of the metal bromide with metal ion in the higher oxidation state entering the bromination to be between 0.5-3M; concentration of the metal bromide with metal ion in the lower oxidation state entering the bromination to be between 0.01-2M; or combinations thereof.

Applicants have found that in order to form the PBH or BE in high space time yield (to minimize reactor costs) with high selectivity (to minimize propylene costs) one or more reaction conditions may be controlled and used. Such one or more reaction conditions include, but are not limited to, temperature and pressure in the bromination reaction; use of the “other DBP” or “other DBE”; use of metal hydroxybromide; amount of salt; amount of total bromide content; amount of metal bromide with metal in the higher oxidation state; amount of metal bromide with metal in the lower oxidation state; residence time of the bromination mixture; presence of a noble metal; etc. The one or more reaction conditions for the bromination reaction/reactor have been described herein.

In some embodiments of all of the aforementioned aspect and embodiments, the PBH or BE is formed with selectivity of between about 20-100%; or between about 20-90%; or between about 20-80%; or between about 20-70%; or between about 20-60%; or between about 20-50%; or between about 20-40%; or between about 30-100%; or between about 30-90%; or between about 30-80%; or between about 30-70%; or between about 30-60%; or between about 30-50%; or between about 30-40%; or between about 40-100%; or between about 40-90%; or between about 40-80%; or between about 40-70%; or between about 40-60%; or between about 40-50%; or between 50-75%; or between about 75-100%; or between about 75-90%; or between about 75-80%; or between about 90-100%; or between about 90-99%; or between about 90-95%. In some embodiments, the above noted selectivity is in wt %.

In some embodiments, the STY (space time yield) of the one or more products from propylene and/or DBP (described further herein below), e.g. the STY of PBH is 0.01, or 0.05, or less than 0.1, or more than 0.1, or more than 0.5, or is 1, or more than 1, or more than 2, or more than 3, or more than 4, or between 0.01-0.05, or between 0.01-0.1, or between 0.1-3, or between 0.5-3, or between 0.5-2, or between 0.5-1, or between 3-5. As used herein the STY is yield per time unit per reactor volume. For example, the yield of product may be expressed in mol, the time unit in hour and the volume in liter and the STY herein are in mol/L/hr. The volume may be the nominal volume of the reactor, e.g. in a packed bed reactor, the volume of the vessel that holds the packed bed is the volume of the reactor. The STY may also be expressed as STY based on the amount of propylene consumed and/or based on amount of the DBP consumed to form the product. For example only, in some embodiments, the STY of the PBH product may be deduced from the amount of propylene consumed and/or based on amount of the DBP consumed during the reaction. The selectivity may be the mol of product, e.g. PBH/mol of the propylene consumed and/or PBH/mol of the DBP consumed. The yield may be the amount of the product recovered. The purity may be the amount of the product/total amount of all products (e.g., amount of PBH/all the organic products formed).

Various other suitable reaction conditions to form PBH or BE have been described herein.

The “other DBP” or “other sources of DBP” as mentioned includes DBP formed as a by-product of other processes. Examples of the other processes or sources include, but are not limited to, the DBP formed by the bromination of the propylene with bromine. The incorporation of this other DBP can lead to additional PBH and PO production by upgrading these streams to more valuable products.

The “other DBE” or “other sources of DBE” as mentioned includes DBE formed as a by-product of other processes. Examples of the other processes or sources include, but are not limited to, the DBE formed by the bromination of the ethylene with bromine. The incorporation of this other DBE can lead to additional BE and EO production by upgrading these streams to more valuable products.

In some embodiments of the aforementioned aspect and embodiments, the methods to form PBH or BE (that may further comprise DBP or DBE, respectively) comprise reaction conditions, such as, but not limited to, use of metal hydroxybromide. Without being limited by any theory, it is contemplated that the metal bromide may react with water and oxygen (e.g. in the oxybromination reaction/reactor) to form metal hydroxybromide species of stoichiometry Mₓⁿ⁺Br_{y}(OH)_{(nx-y)}, MₓBr_{y}(OH)_{(2x-y)}, MₓBr_{y}(OH)_{(3x-y)} or MₓBr_{y}(OH)_{(4x-y)}, where M is the metal ion. An illustration of the reaction is as shown below taking copper bromide as an example:

2CuBr+H₂O+½O₂→2CuBrOH

Where the CuBrOH species represents one of many possible copper hydroxybromide species of stoichiometry CuₓBr_{y}(OH)_{(2x-y)}. If in reaction with e.g. the propylene, the CuBr₂ is replaced (e.g. at least partially) by a hydroxybromide, the following reaction may take place:

C₃H₆(propylene)+CuBrOH+CuBr₂→BrCH₂CH(OH)CH₃(PBH)+2CuBr

This reaction may allow for improved selectivity for the PBH vs. the other products such as the DBP. The reaction with the oxygen to form the metal hydroxybromide species of stoichiometries as noted above, may occur in a reactor separate from the bromination reactor or may occur in the bromination reactor during the bromination of the propylene or may occur in the oxybromination reactor. Other examples of the metal hydroxybromide, without limitation include, MBr(OH)₃, MBr₂(OH)₂, and MBr₃(OH). Similar reaction can take place for ethylene to BE.

In some embodiments of the aforementioned aspect and embodiments, the reaction conditions in the methods to form the PBH or BE comprise brominating a solution containing between about 1-30 wt % salt. The salt may be between 1-30 wt %; or between 1-20 wt % salt; or between 1-5 wt %; or between 5-10 wt %. “Salt” or “saltwater” as used herein includes its conventional sense to refer to a number of different types of salts including, but not limited to, alkali metal bromides such as, sodium bromide, potassium bromide, lithium bromide, cesium bromide, etc.; alkali earth metal bromides such as, calcium bromide, strontium bromide, magnesium bromide, barium bromide, etc; or ammonium bromide. In some embodiments of the foregoing aspects and embodiments, the salt comprises alkali metal bromide and/or alkali earth metal bromide. In some embodiments, the salt (for example only, sodium bromide, or potassium bromide, or lithium bromide, or calcium bromide) in the bromination includes between about 1-30 wt % salt; or between 1-25 wt % salt; or between 1-20 wt % salt; or between 1-10 wt % salt; or between 1-5 wt % salt; or between 5-30 wt % salt; or between 5-20 wt % salt; or between 5-10 wt % salt; or between about 8-30 wt % salt; or between about 8-25 wt % salt; or between about 8-20 wt % salt; or between about 8-15 wt % salt; or between about 10-30 wt % salt; or between about 10-25 wt % salt; or between about 10-20 wt % salt; or between about 10-15 wt % salt; or between about 15-30 wt % salt; or between about 15-25 wt % salt; or between about 15-20 wt % salt; or between about 20-30 wt % salt; or between about 20-25 wt % salt. The salt in water would constitute saltwater as described herein.

In some embodiments, the aqueous medium for the bromination reaction may contain between about 10-80%; or between about 20-80%; or between about 40-80%; or between 40-70%; or between 40-60%; or between 40-50%; or between 50-80%; or between 50-70%; or between 50-60%; or between 60-80%; or between 60-70%; or between 70-80% by weight of water in the aqueous medium depending on the amount of the salt and the metal bromide.

In some embodiments of the aforementioned aspect and embodiments, the reaction conditions in the methods to form the PBH or BE comprise brominating in an aqueous medium with total bromide content of between about 6-40 wt %; or between about 6-30 wt %; or between about 6-20 wt %; or between about 6-10 wt %; or between about 10-30 wt %; or between about 10-20 wt %; or between about 15-30 wt %; or between about 15-20 wt %. The total bromide content is a combination of bromide from the metal bromide (the metal bromide with the metal ion in the lower and the higher oxidation state) as well as the bromide from the salt. Applicants surprisingly observed that bromination in the aqueous medium with total bromide content between about 6-40 wt % resulted in high yield and high selectivity of the PBH or BE over other side products.

In some embodiments of the foregoing aspect and embodiments, reaction conditions for the bromination reaction comprise temperature of the reaction between 40-120° C.; concentration of the metal bromide with metal ion in the higher oxidation state entering the bromination to be between 0.5-3M; concentration of the metal bromide with metal ion in the lower oxidation state entering the bromination to be between 0.01-2M; total bromide content of between about 6-40 wt %; or combinations thereof.

In some embodiments, the reaction conditions in the methods to form the PBH or BE (that may further comprise DBP or DBE, respectively) comprise varying the incubation time or residence time or mean residence time of the bromination mixture. The “incubation time” or “residence time” or “mean residence time” as used herein includes the time period for which the bromination mixture is left in the reactor at the above noted temperatures before being removed. In some embodiments, the residence time for the bromination mixture is a few seconds or between about 1 sec-1 hour; or 1 sec-10 hours; or 10 min-10 hours or more depending on the temperature of the bromination mixture. This residence time may be in combination with other reaction conditions such as, e.g. the temperature ranges and/or total bromide concentrations provided herein. In some embodiments, the residence time for the bromination mixture is between about 1 sec-3 hour; or between about 1 sec-2.5 hour; or between about 1 sec-2 hour; or between about 1 sec-1.5 hour; or between about 1 sec-1 hour; or 1 min-3 hour; or between about 1 min-2.5 hour; or between about 1 min-2 hour; or between about 1 min-1.5 hour; or between about 1 min-1 hour; or between about 1 min-30 min; or between about 2 min-3 hour; or between about 2 min-2 hour; or between about 2 min-1 hour; or between about 3 min-3 hour; or between about 3 min-2 hour; or between about 3 min-1 hour; or between about 5 min-1 hour to form the PBH and/or DBP from the propylene (or the BE and/or DBE from the ethylene) as noted herein.

In some embodiments, the reaction conditions in the methods to form the PBH or BE include carrying out the bromination in the presence of a noble metal. The “noble metal” as used herein includes metals that are resistant to corrosion in moist conditions. In some embodiments, the noble metals are selected from ruthenium, rhodium, palladium, silver, osmium, iridium, platinum, gold, mercury, rhenium, titanium, niobium, tantalum, and combinations thereof. In some embodiments, the noble metal is selected from rhodium, palladium, silver, platinum, gold, titanium, niobium, tantalum, and combinations thereof. In some embodiments, the noble metal is palladium, platinum, titanium, niobium, tantalum, or combinations thereof. In some embodiments, the foregoing noble metals may be present in 0, +2 or +4 oxidation states as appropriate. For example only, platinum or palladium may be present as metal or as a metal over carbon or may be present as PtBr₂ or PdBr₂ etc. In some embodiments, the foregoing noble metal is supported on a solid. Examples of solid support include, without limitation, carbon, zeolite, titanium dioxide, alumina, silica, and the like. In some embodiments, the foregoing noble metal is supported on carbon. For example only, the catalyst is palladium or palladium over carbon. The amount of nobel metal used in the bromination reaction is between 0.001M to 2M; or between 0.001-1.5M; or between about 0.001-1M; or between about 0.001-0.5M; or between about 0.001-0.05M; or between 0.01-2M; or between 0.01-1.5M; or between 0.01-1M; or between 0.01-0.5M; or between 0.1-2M; or between 0.1-1.5M; or between 0.1-1M; or between 0.1-0.5M; or between 1-2M.

In some embodiments of the foregoing aspect and embodiments, the method to form the PBH or BE (that may further comprise DBP or DBE, respectively) further comprises adding platinum or palladium to the aqueous medium. In some embodiments of the foregoing aspect and embodiments, the platinum or palladium is in concentration of between about 0.001-0.1M.

In some embodiments of the foregoing aspects and embodiments, the aqueous medium in the bromination reaction comprises the metal bromide with the metal ion in the higher oxidation state in range of 0.5-3M or 0.5-2M, or 0.5-1M; the metal bromide with the metal ion in the lower oxidation state in range of 0.01-2M, or 0.01-1M, or 0.01-0.5M; and the salt, e.g. sodium or potassium bromide in range of 0.1-5M or 0.1-3M or 0.1-2M or 0.1-1M.

The systems provided herein include the reactor that carries out the bromination, the hydrolysis, the bromine oxidation, the oxybromination, the neutralization, and/or the epoxidation. The “reactor” as used herein is any vessel or unit in which the reaction provided herein, is carried out. The bromination reactor is configured to contact the aqueous medium comprising the metal bromide in the lower and the higher oxidation state and the saltwater from e.g. the anode electrolyte or the saltwater from the oxybromination reaction, with propylene or ethylene to form the one or more products. The oxybromination reactor is configured to contact the metal bromide with the metal ion in the lower oxidation state with the oxidant to form the metal bromide with the metal ion in the higher oxidation state. The reactor may be any means for contacting the contents as mentioned above. Such means or such reactor are well known in the art and include, but not limited to, pipe, column, duct, tank, series of tanks, container, tower, conduit, and the like. The reactor may be equipped with one or more of controllers to control temperature sensor, pressure sensor, control mechanisms, inert gas injector, etc. to monitor, control, and/or facilitate the reaction. Since all the reactors contain aqueous brine, e.g. aq. sodium bromide, the reactors are made from corrosion resistant materials.

In some embodiments, the reactor system may be a series of reactors connected to each other as shown in the figures. The reaction vessel may be a stirred tank. The stirring may increase the mass transfer rate of propylene or ethylene into the aqueous phase accelerating the reaction to form the one or more products. The reactors for the bromination reaction as well as the oxybromination reaction need to be made of material that is compatible with the aqueous or the saltwater streams containing metal ions flowing between the systems. In some embodiments, the electrochemical system, the hydrolysis reactor, the oxidation reactor, the bromination reactor, the neutralization reactor, and/or the oxybromination reactor are made of corrosion resistant materials that are compatible with metal ion containing water, such materials include, titanium, steel etc.

The reactor effluent gases may be quenched with water in the prestressed (e.g., brick-lined) packed tower. The liquid leaving the tower maybe cooled further and separated into the aqueous phase and organic phase. The aqueous phase may be split part being recycled to the tower as quench water and the remainder may be recycled to the reactor or the electrochemical system. The organic product may be cooled further and flashed to separate out more water and dissolved propylene or ethylene. This dissolved propylene or ethylene may be recycled back to the reactor. The uncondensed gases from the quench tower may be recycled to the reactor, except for the purge stream to remove inerts. The purge stream may go through the propylene or ethylene recovery system to keep the over-all utilization of propylene or ethylene high, e.g., as high as 95%. Experimental determinations may be made of flammability limits for propylene or ethylene gas at actual process temperature, pressure and compositions. The construction material of the plant or the systems may include prestressed brick linings, Hastealloys B and C, inconel, dopant grade titanium (e.g. AKOT, Grade II), tantalum, Kynar, Teflon, PEEK, glass, or other polymers or plastics. The reactor may also be designed to continuously flow the anode electrolyte in and out of the reactor.

In some embodiments, the reaction between the metal bromide with metal ion in higher oxidation state and propylene or ethylene is carried out in the reactor provided herein under reaction conditions including, but not limited to, the temperature of between 40-200° C. or between 40-175° C. or between 40-100° C. or between 100-185° C. or between 100-175° C. or between 70-110° C.; pressure of between 10-500 psig or between 10-400 psig or between 10-300 psig or between 10-200 psig or between 10-100 psig or between 50-350 psig or between 200-300 psig, or combinations thereof depending on the desired product. The reactor provided herein is configured to operate at the temperature of between 40-200° C. or between 40-185° C. or between 40-100° C. or between 100-200° C. or between 100-175° C.; pressure of between 10-500 psig or between 10-400 psig or between 10-300 psig or between 50-350 psig or between 200-300 psig, or combinations thereof depending on the desired product. In some embodiments, the reactor provided herein may operate under reaction conditions including, but not limited to, the temperature and pressure in the range of between 35-180° C., or between 35-175° C., or between 40-180° C., or between 40-170° C., or between 40-160° C., or between 50-180° C., or between 50-170° C., or between 50-160° C., or between 55-165° C., or 40° C., or 50° C., or 60° C., or 70° C. and 10-300 psig depending on the desired product. In some embodiments, the reactor provided herein may operate under reaction conditions including, but not limited to, the temperature and pressure in the range of between 35-180° C., or between 35-175° C., or between 40-180° C., or between 40-170° C., or between 40-160° C., or between 50-180° C. and 10-100 psig depending on the desired product.

One or more of the reaction conditions include, such as, but not limited to, temperature of the bromination mixture, incubation time, total bromide concentration in the bromination mixture, and/or concentration of the metal bromide in the higher oxidation state can be set to assure high selectivity, high yield, and/or high STY operation.

Reaction heat may be removed by vaporizing water or by using heat exchange units. In some embodiments, a cooling surface may not be required in the reactor and thus no temperature gradients or close temperature control may be needed.

In some embodiments, the systems may include one reactor or a series of multiple reactors connected to each other or operating separately. The reactor may be a packed bed such as, but not limited to, a hollow tube, pipe, column or other vessel filled with packing material. The reactor may be a trickle-bed reactor. In some embodiments, the packed bed reactor includes a reactor configured such that the aqueous medium containing the metal ions and propylene or ethylene flow counter-currently in the reactor or includes the reactor where the aqueous alkali metal bromide containing the metal ions flows in from the top of the reactor and the propylene or ethylene gas is pressured in from the bottom at e.g., but not limited to, 200 psi or above, such as, for example, 250 psi, 300 psi or 600 psi. In some embodiments, in the latter case, the propylene or ethylene gas may be pressured in such a way that only when the propylene or ethylene gas gets consumed and the pressure drops, that more propylene or ethylene gas flows into the reactor. The trickle-bed reactor includes a reactor where the saltwater such as aqueous alkali metal bromide containing the metal ions and propylene or ethylene flow co-currently in the reactor. In some embodiments, the reactor may be a tray column or a spray tower. Any of the configurations of the reactor described herein may be used to carry out the methods provided herein.

Efficient bromination may be dependent upon achieving intimate contact between the feedstock and the metal bromide in solution and the bromination reaction may be carried out by a technique designed to improve or maximize such contact. The metal ion solution may be agitated by stirring or shaking or any desired technique, e.g. the reaction may be carried out in a column, such as a packed column, or a trickle-bed reactor or reactors described herein. For example, where propylene or ethylene is gaseous, a counter-current technique may be employed wherein the propylene or the ethylene is passed upwardly through a column or reactor and the metal bromide solution is passed downwardly through the column or reactor. In addition to enhancing contact of the propylene or the ethylene and the metal bromide in the solution, the techniques described herein may also enhance the rate of dissolution of the propylene or the ethylene in the solution, as may be desirable in the case where the solution is aqueous and the water-solubility of the propylene or ethylene is low. Dissolution of the feedstock may also be assisted by higher pressures.

A variety of packing material of various shapes, sizes, structure, wetting characteristics, form, and the like may be used in the packed bed or trickle bed reactor, described herein. The packing material includes, but not limited to, polymer (e.g. only Teflon PTFE), ceramic, glass, metal, natural (wood or bark), or combinations thereof. In some embodiments, the packing can be structured packing or loose or unstructured or random packing or combination thereof. The structured packing includes unflowable corrugated metal plates or gauzes. In some embodiments, the structured packing material individually or in stacks fits fully in the diameter of the reactor. The unstructured packing or loose packing or random packing includes flow able void filling packing material.

Examples of loose or unstructured or random packing material include, but not limited to, Raschig rings (such as in ceramic material), pall rings (e.g. in metal and plastic), lessing rings, Michael Bialecki rings (e.g. in metal), berl saddles, intalox saddles (e.g. in ceramic), super intalox saddles, Tellerette® ring (e.g. spiral shape in polymeric material), etc.

Examples of structured packing material include, but not limited to, thin corrugated metal plates or gauzes (honeycomb structures) in different shapes with a specific surface area. The structured packing material may be used as a ring or a layer or a stack of rings or layers that have diameter that may fit into the diameter of the reactor. The ring may be an individual ring or a stack of rings fully filling the reactor. In some embodiments, the voids left out by the structured packing in the reactor are filled with the unstructured packing material.

Examples of structured packing material includes, without limitation, Flexipac®, Intalox®, Flexipac® HC®, etc. In a structured packing material, corrugated sheets may be arranged in a crisscross pattern to create flow channels for the vapor phase. The intersections of the corrugated sheets may create mixing points for the liquid and vapor phases. The structured packing material may be rotated about the column (reactor) axis to provide cross mixing and spreading of the vapor and liquid streams in all directions. The structured packing material may be used in various corrugation sizes and the packing configuration may be optimized to attain the highest efficiency, capacity, and pressure drop requirements of the reactor. The structured packing material may be made of a material of construction including, but not limited to, titanium, stainless steel alloys, carbon steel, aluminum, nickel alloys, copper alloys, zirconium, thermoplastic, etc. The corrugation crimp in the structured packing material may be of any size, including, but not limited to, Y designated packing having an inclination angle of 45° from the horizontal or X designated packing having an inclination angle of 60° from the horizontal. The X packing may provide a lower pressure drop per theoretical stage for the same surface area. The specific surface area of the structured packing may be between 50-800 m²/m³; or between 75-350 m²/m³; or between 200-800 m²/m³; or between 150-800 m²/m³; or between 500-800 m²/m³.

In some embodiments, the structured or the unstructured packing material as described above is used in the distillation or flash column described herein for separation and purification of the products.

### Bromination to Form DBP and Hydrolyze DBP to PBH and Propanal or to Form DBE and Hydrolyze DBE to BE and Optionally Bromoacetaldehyde

As described above, DBP may be another product formed after the bromination of propylene. The “1,2-dibromopropane” or “dibromopropane” or “propylene dibromide” or “DBP” or “PDB” can be used interchangeably herein. Similarly, dibromoethane (DBE) may be another product formed after the bromination of ethylene. The “1,2-dibromoethane” or “dibromoethane” or “ethylene dibromide” or “DBE” or “EDB” can be used interchangeably herein.

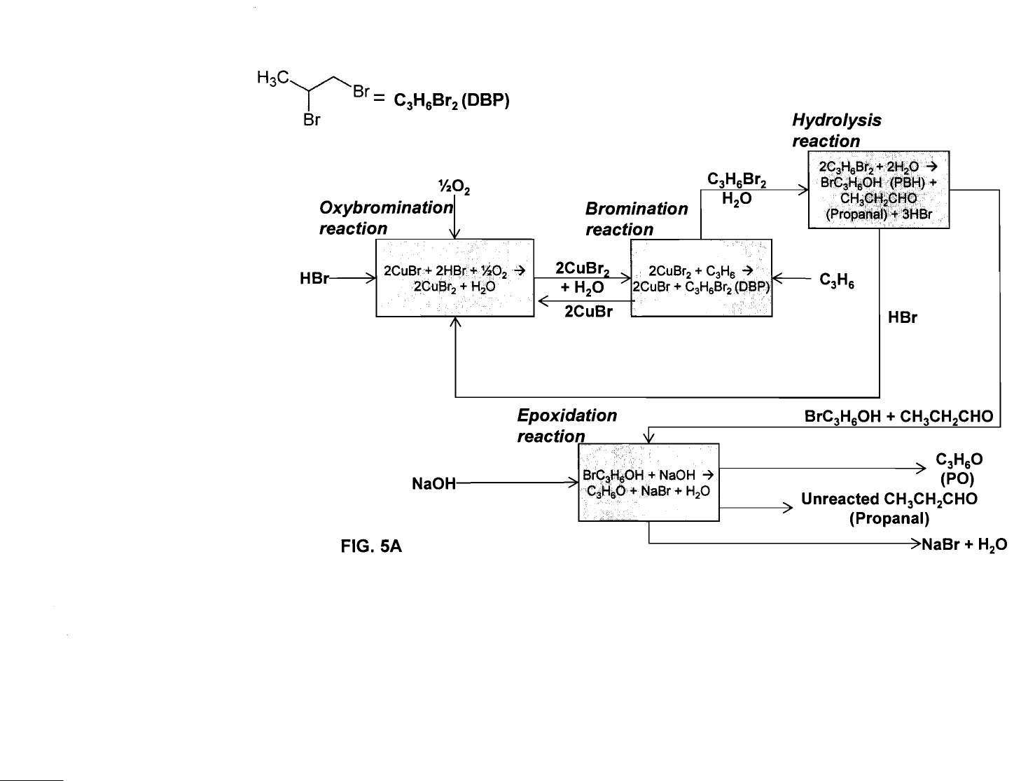

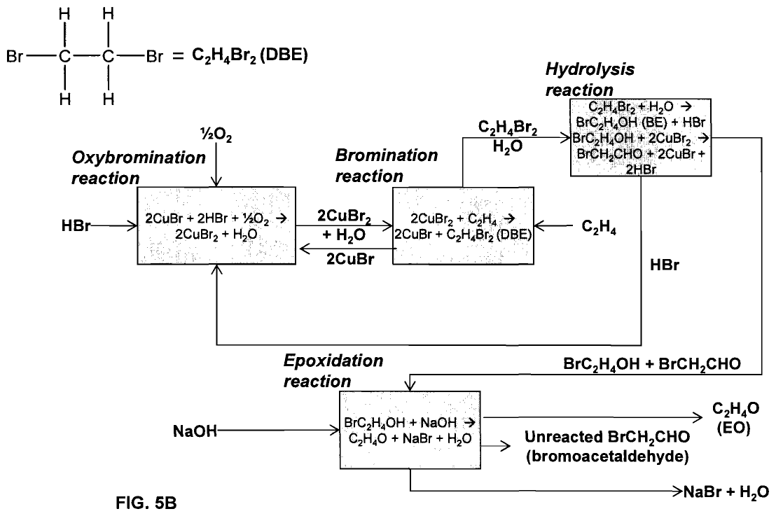

In some embodiments, there are provided methods and systems to convert the DBP to the PBH or the DBE to BE in the same or a separate reactor. In some embodiments, the DBP or DBE may be formed as a side product and in one aspect, there are provided methods and systems to convert the DBP to the PBH or the DBE to BE in the same or a separate reactor. The hydrolysis reactions have been illustrated in FIGS. 1A, 1B, 5A, and 5B.

In some embodiments, the conversion of the DBP to the PBH is a hydrolysis reaction:

BrCH₂CH(Br)CH₃+H₂O→BrCH₂CH(OH)CH₃+HBr

BrCH₂CH(Br)CH₃+H₂O→HOCH₂CH(Br)CH₃+HBr

In reactions above, the DBP is hydrolyzed by water into two isomers of the PBH: 1-bromo-2-propanol and 2-bromo-1-propanol. The conversion of the DBP to the PBH is slow at room temperature. In some embodiments, there are provided efficient methods to convert the DBP to the PBH by hydrolysis.

As described earlier, the amount of DBP formed from the propylene in the bromination reaction/reactor may be considerably lower and the amount of PBH may be considerably higher compared to the metal chloride methods and systems where higher amount of dichloropropane is formed. It has been observed that the conversion and selectivity of the reaction transforming the DBP to the PBH is higher than the conversion and selectivity of the reaction transforming the dichloropropane to the PCH. For example, the DBP to the PBH yields a selectivity of approximately 75% or more; 80% or more; 85% or more; 90% or more; or 92%; or between 70-95%; or between 75-85%; or between 90-95%; or between 90-99%; or between 95-99%. Furthermore, in addition to the improved selectivity, it has been found that the DBP to PBH formation reaction could be performed using the metal bromide solution as the catalytic solution rather than requiring a second catalyst system, which significantly reduces process complexity especially with regard to the recovery and reuse of the resulting acid HBr.

In one aspect, there are provided methods that include brominating propylene in an aqueous medium comprising metal bromide with metal ion in higher oxidation state, metal bromide with metal ion in lower oxidation state, and saltwater under reaction conditions to result in one or more products comprising DBP, and the metal bromide with the metal ion in lower oxidation state; and hydrolyzing the DBP under one or more reaction conditions to result in hydrolysis products comprising PBH and propanal (as illustrated in FIGS. 1A and 5A, propanal or CH₃CH₂CHO is illustrated in FIG. 1A). In some embodiments of the foregoing aspect, the method further comprises epoxidizing the hydrolysis products comprising PBH and propanal to PO and unreacted propanal. In some embodiments of the foregoing aspect and embodiments, the unreacted propanal is isolated from the PO.

In some embodiments of the foregoing aspect, the one or more products further comprise PBH. In some embodiments of the aforementioned embodiment, the methods comprise brominating propylene with an aqueous medium comprising metal bromide with metal ion in higher oxidation state, metal bromide with metal ion in lower oxidation state, and saltwater to result in one or more products comprising DBP and PBH and reduction of the metal bromide with the metal ion in the higher oxidation state to the metal bromide with the metal ion in the lower oxidation state; epoxidizing the one or more products comprising DBP and PBH with a base to form PO and unreacted DBP; and hydrolyzing the unreacted DBP under one or more reaction conditions to result in hydrolysis products comprising PBH and propanal. This embodiment is illustrated in FIG. 1A.

In one aspect, there are provided methods that include brominating ethylene in an aqueous medium comprising metal bromide with metal ion in higher oxidation state, metal bromide with metal ion in lower oxidation state, and saltwater under reaction conditions to result in one or more products comprising DBE, and the metal bromide with the metal ion in lower oxidation state; and hydrolyzing the DBE under one or more reaction conditions to result in hydrolysis products comprising BE and optionally bromoacetaldehyde (illustrated in FIGS. 1B and 5B, bromoacetaldehyde is illustrated in FIG. 1B). In some embodiments of the foregoing aspect, the method further comprises epoxidizing the hydrolysis products comprising BE and optionally bromoacetaldehyde to EO and unreacted bromoacetaldehyde. In some embodiments of the foregoing aspect and embodiments, the unreacted bromoacetaldehyde is isolated from the EO.

In some embodiments of the foregoing aspect, the one or more products further comprise BE. In some embodiments, the methods comprise brominating ethylene with an aqueous medium comprising metal bromide with metal ion in higher oxidation state, metal bromide with metal ion in lower oxidation state, and saltwater to result in one or more products comprising DBE and BE and reduction of the metal bromide with the metal ion in the higher oxidation state to the metal bromide with the metal ion in the lower oxidation state; epoxidizing the one or more products comprising DBE and BE with a base to form EO and unreacted DBE; and hydrolyzing the unreacted DBE under one or more reaction conditions to result in hydrolysis products comprising BE and optionally bromoacetaldehyde (BrCH₂CHO). This embodiment is illustrated in FIG. 1B.

In some embodiments of the foregoing aspect and embodiments, the method comprises one or more of (A) hydrolyzing the DBP to the PBH in situ; and/or (B) separating the DBP from the aqueous medium and/or from the PBH (when both DBP and PBH are formed in the bromination reaction) and hydrolyzing the DBP to the PBH and the propanal and/or epoxidizing the PBH to PO; and/or (C) hydrolyzing the DBP to the PBH and the propanal without the separation of the DBP from the PBH and/or from the aqueous medium, to increase the yield of the PBH.

In some embodiments of the foregoing aspect and embodiments, the method comprises one or more of (A) hydrolyzing the DBE to the BE in situ; and/or (B) separating the DBE from the aqueous medium and/or from the BE (when both DBE and BE are formed in the bromination reaction) and hydrolyzing the DBE to the BE and optionally bromoacetaldehyde and/or epoxidizing the BE to EO; and/or (C) hydrolyzing the DBE to the BE and optionally bromoacetaldehyde without the separation of the DBE from the BE and/or from the aqueous medium, to increase the yield of the BE.

In some embodiments of the systems described herein, the system further comprises a hydrolyzing chamber (configured to carry out the hydrolysis as described in the aforementioned methods) operably connected to the bromination reactor and configured to receive the DBP or DBE from the bromination reactor and hydrolyze the DBP to PBH and the propanal or hydrolyze the DBE to BE and optionally bromoacetaldehyde (illustrated in FIGS. 1A, 5A, 1B, and 5B).

In some embodiments, the hydrolyzing chamber is also operably connected to the epoxide reactor (as shown in FIGS. 1A, 5A, 1B, and 5B) and is configured to transfer the PBH and the propanal or the BE and optionally bromoacetaldehyde (and other bromo derivatives as described herein) to the epoxide reactor to form PO or EO, respectively. In the aforementioned embodiment, the hydrolyzing chamber or reactor may be connected to a separation chamber before connecting to the epoxide reactor such that the organics comprising the PBH and the propanal or the BE and optionally bromoacetaldehyde is separated from the aqueous medium before transferring the organics to the epoxide reactor. In some embodiments, the hydrolyzing chamber is operably connected to the epoxide reactor and is configured to receive the unreacted DBP or the unreacted DBE from the epoxide reactor. For example, in some embodiments, the DBP is used as an extraction solvent (described further herein) to extract the PBH from the aqueous solution after the bromination reaction. In such embodiments, the epoxidation is carried out by mixing the DBP solvent (containing the PBH) with NaOH. After the epoxidation reaction of the PBH to the PO, the unreacted DBP may be sent to the hydrolyzing chamber for the hydrolysis reaction of the DBP to the PBH and the propanal, before sending the PBH and the propanal back to the epoxide reactor (DBP “loop”). The DBP circulated from the epoxide reactor/reaction to the hydrolyzing reactor/reaction provides an efficient source of DBP as this DBP has minimum side products or the PBH.

In some embodiments of the above noted system, the system further comprises means for transferring HBr formed in the hydrolyzing chamber to the oxybromination reactor. Such means include any means for transferring liquids including, but not limited to, conduits, tanks, pipes, and the like.

The bromination reaction may take place after the electrochemical reaction and/or the oxybromination reaction (described further herein). Accordingly, in some embodiments there are provided methods that include (i) contacting an anode with an anode electrolyte in an electrochemical cell wherein the anode electrolyte comprises metal bromide with metal ion in lower oxidation state, metal bromide with metal ion in higher oxidation state, and saltwater; contacting a cathode with a cathode electrolyte in the electrochemical cell; applying voltage to the anode and the cathode and oxidizing the metal bromide with the metal ion in the lower oxidation state to the higher oxidation state at the anode; (ii) withdrawing the anode electrolyte from the electrochemical cell and brominating propylene with the anode electrolyte (also called aqueous medium) comprising the metal bromide with the metal ion in the higher oxidation state in the saltwater to result in one or more products comprising DBP and the metal bromide with the metal ion in the lower oxidation state; and (iii) hydrolyzing the DBP under one or more reaction conditions to result in hydrolysis products comprising PBH and propanal. In some embodiments of the foregoing aspect and embodiments, the one or more products further comprise PBH. In some embodiments of the foregoing aspect and embodiments, the method further comprises epoxidizing the hydrolysis products comprising PBH and propanal to form PO and unreacted propanal.

In some embodiments, there are provided methods that include (i) oxidizing metal bromide with metal ion in a lower oxidation state to a higher oxidation state in presence of an oxidant in an oxybromination reaction; (ii) withdrawing the metal bromide with metal ion in the higher oxidation state from the oxybromination reaction and brominating propylene with the metal bromide with metal ion in the higher oxidation state in saltwater to result in one or more products comprising DBP and the metal bromide with the metal ion in the lower oxidation state; and (iii) hydrolyzing the DBP under one or more reaction conditions to result in hydrolysis products comprising PBH and propanal. In some embodiments of the foregoing aspect and embodiments, the one or more products further comprise PBH. In some embodiments of the foregoing aspect and embodiments, the method further comprises epoxidizing the hydrolysis products comprising PBH and propanal to form PO and unreacted propanal.

In some embodiments there are provided methods that include (i) contacting an anode with an anode electrolyte in an electrochemical cell wherein the anode electrolyte comprises metal bromide with metal ion in lower oxidation state, metal bromide with metal ion in higher oxidation state, and saltwater; contacting a cathode with a cathode electrolyte in the electrochemical cell; applying voltage to the anode and the cathode and oxidizing the metal bromide with the metal ion in the lower oxidation state to the higher oxidation state at the anode; (ii) withdrawing the anode electrolyte from the electrochemical cell and brominating propylene with the anode electrolyte (also called aqueous medium) comprising the metal bromide with the metal ion in the higher oxidation state in the saltwater to result in one or more products comprising DBP and PBH and the metal bromide with the metal ion in the lower oxidation state; (iii) epoxidizing the one or more products comprising DBP and PBH with a base to form PO and unreacted DBP; and (iv) hydrolyzing the unreacted DBP under one or more reaction conditions to result in hydrolysis products comprising PBH and propanal.

In some embodiments, there are provided methods that include (i) oxidizing metal bromide with metal ion in a lower oxidation state to a higher oxidation state in presence of an oxidant in an oxybromination reaction; (ii) withdrawing the metal bromide with metal ion in the higher oxidation state from the oxybromination reaction and brominating propylene with the metal bromide with metal ion in the higher oxidation state in saltwater to result in one or more products comprising DBP and PBH and the metal bromide with the metal ion in the lower oxidation state; (iii) epoxidizing the one or more products comprising DBP and PBH with a base to form PO and unreacted DBP; and (iv) hydrolyzing the unreacted DBP under one or more reaction conditions to result in hydrolysis products comprising PBH and propanal.

In some embodiments of the foregoing aspect and embodiments, the method further comprises one or more of (A) hydrolyzing the DBP to the PBH in situ; and/or (B) separating the DBP from the aqueous medium and/or from the PBH and then hydrolyzing the DBP to the PBH; and/or (C) hydrolyzing the DBP to the PBH without the separation of the DBP from the PBH and/or the aqueous medium, to increase the yield of the PBH. In some embodiments of the aforementioned embodiments, the method further includes returning the saltwater (e.g. aq. NaBr) from the epoxidation reaction/reactor to the electrochemical reaction/cell and/or to the oxybromination reaction/reactor.

Accordingly, in some embodiments there are provided methods that include (i) contacting an anode with an anode electrolyte in an electrochemical cell wherein the anode electrolyte comprises metal bromide with metal ion in lower oxidation state, metal bromide with metal ion in higher oxidation state, and saltwater; contacting a cathode with a cathode electrolyte in the electrochemical cell; applying voltage to the anode and the cathode and oxidizing the metal bromide with metal ion in a lower oxidation state to a higher oxidation state at the anode; (ii) withdrawing the anode electrolyte from the electrochemical cell and brominating ethylene with the anode electrolyte (also called aqueous medium) comprising the metal bromide with the metal ion in the higher oxidation state in the saltwater to result in one or more products comprising DBE and the metal bromide with the metal ion in the lower oxidation state; and (iii) hydrolyzing the DBE under one or more reaction conditions to result in hydrolysis products comprising BE and bromoacetaldehyde.

In some embodiments, there are provided methods that include (i) oxidizing metal bromide with metal ion in a lower oxidation state to a higher oxidation state in presence of an oxidant in an oxybromination reaction; (ii) withdrawing the metal bromide with metal ion in the higher oxidation state from the oxybromination reaction and brominating ethylene with the metal bromide with metal ion in the higher oxidation state in saltwater to result in one or more products comprising DBE and the metal bromide with the metal ion in the lower oxidation state; and (iii) hydrolyzing the DBE under one or more reaction conditions to result in hydrolysis products comprising BE and bromoacetaldehyde. In some embodiments of the foregoing aspect and embodiments, the one or more products from bromination further comprise BE.

In some embodiments there are provided methods that include (i) contacting an anode with an anode electrolyte in an electrochemical cell wherein the anode electrolyte comprises metal bromide with metal ion in lower oxidation state, metal bromide with metal ion in higher oxidation state, and saltwater; contacting a cathode with a cathode electrolyte in the electrochemical cell; applying voltage to the anode and the cathode and oxidizing the metal bromide with the metal ion in the lower oxidation state to the higher oxidation state at the anode; (ii) withdrawing the anode electrolyte from the electrochemical cell and brominating ethylene with the anode electrolyte (also called aqueous medium) comprising the metal bromide with the metal ion in the higher oxidation state in the saltwater to result in one or more products comprising DBE and BE and the metal bromide with the metal ion in the lower oxidation state; (iii) epoxidizing the one or more products comprising DBE and BE with a base to form EO and unreacted DBE; and (iv) hydrolyzing the unreacted DBE under one or more reaction conditions to result in hydrolysis products comprising BE and bromoacetaldehyde.

In some embodiments, there are provided methods that include (i) oxidizing metal bromide with metal ion in a lower oxidation state to a higher oxidation state in presence of an oxidant in an oxybromination reaction; (ii) withdrawing the metal bromide with metal ion in the higher oxidation state from the oxybromination reaction and brominating ethylene with the metal bromide with metal ion in the higher oxidation state in saltwater to result in one or more products comprising DBE and BE and the metal bromide with the metal ion in the lower oxidation state; (iii) epoxidizing the one or more products comprising DBE and BE with a base to form EO and unreacted DBE; and (iv) hydrolyzing the unreacted DBE under one or more reaction conditions to result in hydrolysis products comprising BE and bromoacetaldehyde.

In some embodiments of the foregoing aspect and embodiments, the method further comprises one or more of (A) hydrolyzing the DBE to the BE in situ; and/or (B) separating the DBE from the aqueous medium and/or from the BE and then hydrolyzing the DBE to the BE; and/or (C) hydrolyzing the DBE to the BE without the separation of the DBE from the BE and/or the aqueous medium, to increase the yield of the BE. In some embodiments of the aforementioned embodiments, the method further includes returning the saltwater (e.g. aq. NaBr) from the epoxidation reaction/reactor to the electrochemical reaction/cell and/or to the oxybromination reaction/reactor.

Applicants surprisingly observed that the separation or without separation of the DBP from the aqueous medium or the separation or without separation of the DBE from the aqueous medium had a significant effect on the products formed after hydrolysis (Examples 5, 6, and 7).

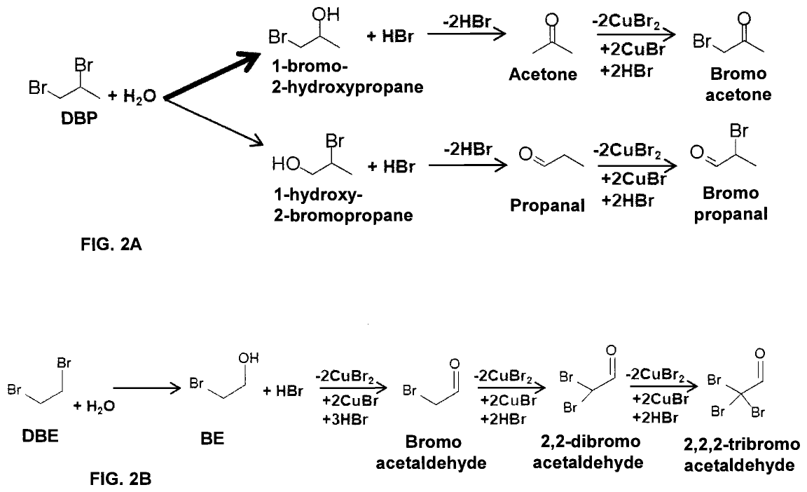

FIGS. 2A and 2B illustrate formation of various hydrolysis products depending on the absence or presence of metal bromide and salts in the reaction. As is illustrated in FIG. 2A, it is contemplated that the hydrolysis of DBP results in the formation of two isomers of PBH (1-bromo-2-hydroxy propane and 1-hydroxy-2-bromo propane) which undergo further elimination of HBr to form acetone and propanal. Without being limited by any theory, it is to be understood that while 1-bromo-2-hydroxy propane is shown to form acetone, the 1-hydroxy-2-bromo propane may undergo rearrangement and result in the formation of acetone or vice versa. FIGS. 2A and 2B illustrate only one of the routes for the formation of the products. As such, all the mechanisms to form acetone and propanal from the PBH are within the scope of the disclosure. In some embodiments, when the DBP is not separated from the aqueous medium comprising metal bromide and salt, the presence of the metal bromide in the hydrolysis reaction may further facilitate formation of bromo derivatives and result in bromoacetone and/or bromopropanal. The bromoacetone may further undergo bromination to form dibromo and/or tribromo acetone. The bromopropanal may further undergo bromination to form dibromopropanal and/or tribromopropanal.

Similarly, as is illustrated in FIG. 2B, the hydrolysis of DBE results in the formation of BE which undergoes oxidation in the presence of metal salts to form bromoacetaldehyde, dibromoacetaldehyde, and/or tribromoacetaldehyde.

Applicants observed that the hydrolysis reaction and the product formation may be affected by organic:aqueous ratio in the hydrolysis reaction. Based on the observations, the reaction may occur, if not entirely, in the aqueous phase of the reaction. As a result, the amount of PBH may increase as the amount of water increases (and the amount of DBP decreases at constant volume). As shown in the Examples 5 or 6 herein, the amount of propanal and acetone increases with a decrease in the organic:aqueous ratio. As a result, the organic:aqueous ratio may be used as a means to selectively produce propanal and/or acetone.

In some embodiments of the aspects provide herein, the one or more reaction conditions in the hydrolysis reaction comprise organic:aqueous ratio between 0.5:10-10:0.5; or between 0.5:8-8:0.5; or between 0.5:6-6:0.5; or between 0.5:5-5:0.5; or between 0.5:4-4:0.5; or between 0.5:3-3:0.5; or between 0.5:2-2:0.5; or between 0.5:1-1:0.5; or between 2:1-1:2; or between 3:1-1:3 or 5:1 or 4:1 or 3:1 or 2:1 or 1.5:1 or 1:1.

In one aspect, there is provided a system comprising (i) an electrochemical cell comprising an anode chamber comprising an anode and an anode electrolyte wherein the anode electrolyte comprises metal bromide with metal ion in a lower oxidation state, metal bromide with metal ion in a higher oxidation state, and saltwater and anode is configured to oxidize the metal bromide with the metal ion in the lower oxidation state to the higher oxidation state; a cathode chamber comprising a cathode and a cathode electrolyte; and a voltage source configured to apply voltage to the anode and the cathode; (ii) a bromination reactor operably connected to the anode chamber of the electrochemical cell and configured to obtain the anode electrolyte and brominate propylene with the anode electrolyte comprising the metal bromide with the metal ion in the higher oxidation state in the saltwater to result in one or more products comprising DBP and PBH and the metal bromide with the metal ion in the lower oxidation state; (iii) a hydrolysis reactor operably connected to the bromination reactor and/or an epoxidation reactor and configured to obtain the one or more products comprising DBP and PBH from the bromide reactor and/or unreacted DBP from the epoxidation reactor, with or without the saltwater comprising metal bromide configured to hydrolyze the DBP to the PBH and propanal; and (iv) an epoxidation reactor operably connected to the hydrolysis reactor and configured to obtain the solution comprising PBH and propanal and epoxidize the PBH to PO and unreacted propanal in presence of a base and/or an epoxidation reactor operably connected to the bromination reactor and configured to obtain the solution comprising DBP and PBH and epoxidize the PBH to PO and unreacted DBP in presence of a base. In some embodiments, the system further comprises an oxybromination reactor operably connected to the bromination reactor and/or the electrochemical cell, and the hydrolysis reactor and configured to obtain aqueous medium from the bromination reactor and/or the electrochemical cell comprising the metal bromide with metal ion in the lower oxidation state and the higher oxidation state and obtain HBr produced in the hydrolysis reactor and is configured to oxidize the metal bromide with metal ion in the lower oxidation state to the higher oxidation state using an oxidant comprising the HBr and oxygen, or hydrogen peroxide (or any other oxidant as described herein). In some embodiments, the system further comprises the epoxidation reactor operably connected to the electrochemical cell.

In one aspect, there is provided a system comprising (i) an electrochemical cell comprising an anode chamber comprising an anode and an anode electrolyte wherein the anode electrolyte comprises metal bromide with metal ion in a lower oxidation state, metal bromide with metal ion in a higher oxidation state, and saltwater and anode is configured to oxidize the metal bromide with the metal ion in the lower oxidation state to the higher oxidation state; a cathode chamber comprising a cathode and a cathode electrolyte; and a voltage source configured to apply voltage to the anode and the cathode; (ii) a bromination reactor operably connected to the anode chamber of the electrochemical cell and configured to obtain the anode electrolyte and brominate ethylene with the anode electrolyte comprising the metal bromide with the metal ion in the higher oxidation state in the saltwater to result in one or more products comprising DBE and BE and the metal bromide with the metal ion in the lower oxidation state; (iii) a hydrolysis reactor operably connected to the bromination reactor and/or an epoxidation reactor and configured to obtain the one or more products comprising DBE and BE from the bromide reactor and/or unreacted DBE from the epoxidation reactor, with or without the saltwater comprising metal bromide configured to hydrolyze the DBE to BE and optionally bromoacetaldehyde; and (iv) an epoxidation reactor operably connected to the hydrolysis reactor and configured to obtain the solution comprising BE and optionally bromoacetaldehyde and epoxidize the BE to EO and optionally unreacted bromoacetaldehyde in presence of a base and/or an epoxidation reactor operably connected to the bromination reactor and configured to obtain the solution comprising DBE and BE and epoxidize the BE to EO and unreacted DBE in presence of a base. In some embodiments, the system further comprises an oxybromination reactor operably connected to the bromination reactor and/or the electrochemical cell, and the hydrolysis reactor and configured to obtain aqueous medium from the bromination reactor and/or the electrochemical cell comprising the metal bromide with metal ion in the lower oxidation state and the higher oxidation state and obtain HBr produced in the hydrolysis reactor and is configured to oxidize the metal bromide with metal ion in the lower oxidation state to the higher oxidation state using an oxidant comprising the HBr and oxygen, or hydrogen peroxide (or any other oxidant as described herein). In some embodiments, the system further comprises the epoxidation reactor operably connected to the electrochemical cell.

In one aspect, the oxybromination reactor is used independent of the electrochemical cell (as illustrated in FIGS. 5A and 5B). In some embodiments, there is provided a system comprising (i) oxybromination reactor configured to oxidize metal bromide with metal ion in lower oxidation state to higher oxidation state using an oxidant comprising oxygen or hydrogen peroxide and optionally HBr (or any other oxidant as described herein); (ii) a bromination reactor operably connected to the oxybromination reactor and configured to obtain the metal bromide with the metal ion in the higher oxidation state and brominate propylene or ethylene with the metal bromide with the metal ion in the higher oxidation state in saltwater to result in one or more products comprising DBP or DBE, respectively, and the metal bromide with the metal ion in the lower oxidation state; (iii) a hydrolysis reactor operably connected to the bromination reactor and configured to obtain the one or more products comprising DBP or DBE from the bromination reactor with or without the saltwater comprising metal bromide and configured to hydrolyze the DBP to the PBH and propanal or the DBE to the BE and optionally bromoacetaldehyde; and (iv) an epoxidation reactor operably connected to the hydrolysis reactor and configured to obtain the solution comprising PBH and propanal or BE and optionally bromoacetaldehyde and epoxidize the PBH to PO and unreacted propanal or BE to EO and optionally unreacted bromoacetaldehyde, in presence of a base. In some embodiments, the oxybromination reactor is also operably connected to the bromination reactor and the hydrolysis reactor and is configured to obtain the aqueous medium from the bromination reactor comprising the metal bromide with metal ion in the lower oxidation state and the higher oxidation state and is optionally configured to obtain HBr produced in the hydrolysis reactor.

In some embodiments of the aforementioned embodiments, the bromination reactor may be operably connected to the epoxide reactor directly (as shown in the FIGS. 1A and 1B) and is configured to transfer the one or more products comprising PBH and DBP or BE and DBE to the epoxide reactor to epoxidize the PBH and DBP to PO and unreacted DBP, or BE and DBE to EO and unreacted DBE, respectively, in presence of the base. The epoxide reactor may in turn be operably connected to the hydrolysis reactor to transfer the unreacted DBP or the unreacted DBE to the hydrolysis reactor (DBP loop or DBE loop, as described herein) for hydrolysis. The unreacted propanal may be isolated and commercially sold.

Therefore, any number of combinations of the electrochemical cell/reaction, oxybromination reactor/reaction, bromination reactor/reaction, hydrolysis reactor/reaction, and epoxide reactor/reactions are possible and are within the scope of the invention.

In some embodiments, the reaction conditions listed in the foregoing section also aid in (A) the hydrolysis of the DBP to the PBH and optionally propanal in situ (e.g. during bromination reaction in the bromination reactor). The DBP may be hydrolyzed to the PBH in situ by increasing the available free water during the reaction. Because water is a reactant in the hydrolysis of the DBP to the PBH and propanal, the presence of free water may lead to the conversion of the DBP to the PBH and propanal during the bromination.

In some embodiments, the DBP may be formed in high yield and may then be hydrolyzed to the PBH and propanal. In such embodiments, some amount of PBH may be formed in the bromination reaction which may or may not be separated from the DBP. There may be a number of options to increase the rate and/or selectivity of the DBP formation. These options include highly concentrated salt solutions which reduce the available free water. Because water is a reactant in the hydrolysis of the DBP to the PBH and propanal, the presence of free water may lead to the conversion of the DBP to the PBH and propanal. The high concentrations of salt may be accomplished through the addition of the copper bromide salts (such as CuBr₂, CuBr or in combination) or through other salts such as NaBr. There are also a number of process conditions which can be optimized to provide higher STY and better selectivity for the DBP or DBE production including temperature, pressure (e.g. pressures under which the propylene may form a liquid or supercritical phase), and residence time.

In one aspect, the conversion of the DBP to the PBH and propanal may be executed in a second reaction step downstream (in a separate reactor) of the propylene bromination, illustrated as the hydrolysis reactor in FIGS. 1A and 5A. The DBP may be hydrolyzed to the PBH and propanal by (B) separating the DBP from the aqueous medium and/or from the PBH (when both DBP and PBH are formed in the bromination reaction) and then hydrolyzing the DBP to the PBH and propanal; and/or (C) hydrolyzing the DBP to the PBH and propanal without the separation of the DBP from the PBH and/or the aqueous medium, to increase the yield of the PBH. When the hydrolysis is done in a second step, the hydrolysis of the DBP to the PBH and propanal may utilize the aqueous stream leaving the bromination reaction/reactor (containing the aqueous metal bromide, e.g. aqueous copper bromide) as part of a circulating loop (embodiment C above related to hydrolysis without the separation of the DBP from the aqueous medium). Illustrated in FIGS. 1A, 1B, 5A, and 5B is the aspect where the DBP is converted to the PBH and propanal or the DBE is converted to the BE and bromoacetaldehyde in a hydrolysis reaction/reactor after the bromination reaction/reactor.

To leverage the process economics of the conversion of the DBP to the PBH and propanal in an optimum way, the process may recover at least some of the HBr by-product from the hydrolysis of the DBP to the PBH and propanal. This HBr can be reused in the oxybromination unit within the process to generate additional PO.

In some embodiments, use of Lewis acid in the hydrolysis reaction can result in high yield and high selectivity of the PBH from the DBP or the BE from the DBE. The “Lewis acid” as used herein includes any conventional Lewis acid capable of accepting an electron pair. Without limitation, Lewis acids herein include hard acids and soft acids. Examples include, but are not limited to, silicon bromide, e.g. SiBr₄; germanium bromide, e.g. GeBr₄; tin bromide, e.g. SnBr₄; boron bromide, e.g. BBr₃; aluminum bromide, e.g. AlBr₃; gallium bromide, e.g. GaBr₃; indium bromide, e.g. InBr₃; thallium bromide, e.g. TlBr₃; phosphorus bromide, e.g. PBr₃; antimony bromide, e.g. SbBr₃; arsenic bromide, e.g. AsBr₃; copper bromide, e.g. CuBr₂; zinc bromide, e.g. ZnBr₂; titanium bromide, e.g. TiBr₃ or TiBr₄; vanadium bromide, e.g. VBr₄; chromium bromide, e.g. CrBr₂; manganese bromide, e.g. MnBr₂; iron bromide, e.g. FeBr₂ or FeBr₃; cobalt bromide, e.g. CoBr₂; or nickel bromide, e.g. NiBr₂. The Lewis acid also includes, but is not limited to, lanthanide bromide selected from lanthanum bromide, cerium bromide, praseodymium bromide, neodymium bromide, promethium bromide, samarium bromide, europium bromide, gadolinium bromide, terbium bromide, dysprosium bromide, holmium bromide, erbium bromide, thulium bromide, ytterbium bromide, or lutetium bromide. The Lewis acid also includes, but is not limited to, triflates, e.g. scandium triflate, e.g. Sc(OTf)₃ or zinc triflate, e.g. Zn(OTf)₂—where Tf=triflate; SO₃CF₃.

In some embodiments, the Lewis acid is selected from silicon bromide; germanium bromide; tin bromide; boron bromide; aluminum bromide; gallium bromide; indium bromide; thallium bromide; phosphorus bromide; antimony bromide; arsenic bromide; copper bromide; zinc bromide; titanium bromide; vanadium bromide; chromium bromide; manganese bromide; iron bromide; cobalt bromide; nickel bromide; lanthanide bromide; and triflate. In some embodiments, the Lewis acid is selected from SiBr₄; GeBr₄; SnBr₄; BBr₃; AlBr₃; GaBr₃; InBr₃; TlBr₃; PBr₃; SbBr₃; AsBr₃; CuBr₂; ZnBr₂; TiBr₃; TiBr₄; VBr₄; CrBr₂; MnBr₂; FeBr₂; FeBr₃; CoBr₂; NiBr₂; LaBr₃; Zn(OTf)₂; and Sc(OTf)₃. In some embodiments, the Lewis acid is selected from BBr₃; AlBr₃; GaBr₃; InBr₃; TlBr₃; CuBr₂; ZnBr₂; SnBr₄; TiBr₃; TiBr₄; and LaBr₃. In some embodiments, the Lewis acid is AlBr₃; GaBr₃; CuBr₂; SnBr₄; or ZnBr₂. In some embodiments, the Lewis acid is ZnBr₂ or SnBr₄.

In some embodiments, the Lewis acid may be replaced by Bronsted acid for the hydrolysis of the DBP to the PBH or DBE to BE. The “Bronsted acid” as used herein, includes any compound that can transfer a proton to any other compound. Examples of the Bronsted acid, include, but are not limited to, heteropoly acids, such has, H₃PMo₁₂O₄₀; H₃PW₁₂O₄₀; H₃PMo₆V₆O₄₀; H₄XM₁₂O₄₀ where X=Si or Ge and M=Mo or W; H₃XM₁₂O₄₀ where X=P or As and M=Mo or W; or H₆X₂M₁₈O₆₂ where X=P or As and M=Mo or W. The symbols of the chemical elements are well known in the art. All the aspects and embodiments related to the Lewis acid can be applied to the Bronsted acid and as such all are within the scope of the invention.

In some embodiments of the foregoing aspect and embodiments, the Lewis acid herein is used as an aqueous solution of the Lewis acid. Accordingly, in some embodiments of the foregoing aspects and embodiments, there are provided methods to form PBH, comprising: hydrolyzing DBP to PBH in an aqueous solution comprising Lewis acid. There are also provided methods to form PBH and propanal, comprising: hydrolyzing DBP to PBH and propanal in an aqueous solution comprising Lewis acid. There are also provided methods to form BE, comprising: hydrolyzing DBE to BE in an aqueous solution comprising Lewis acid. There are also provided methods to form BE and bromoacetaldehyde, comprising: hydrolyzing DBE to BE and bromoacetaldehyde in an aqueous solution comprising Lewis acid. In some embodiments, the Lewis acid concentration is in a range of about 0.1-6 mol/kg of the solution. In some embodiments, the Lewis acid is in a concentration in a range of about 0.1-6 mol/kg; or about 0.1-5.5 mol/kg; or about 0.1-5 mol/kg; or about 0.1-4.5 mol/kg; or about 0.1-4 mol/kg; or about 0.1-3.5 mol/kg; or about 0.1-3 mol/kg; or about 0.1-2.5 mol/kg; or about 0.1-2 mol/kg; or about 0.1-1.5 mol/kg; or about 0.1-1 mol/kg; or about 0.1-0.5 mol/kg; or about 0.5-6 mol/kg; or about 0.5-5 mol/kg; or about 0.5-4 mol/kg; or about 0.5-3 mol/kg; or about 0.5-2 mol/kg; or about 0.5-1 mol/kg; or about 1-6 mol/kg; or about 1-5 mol/kg; or about 1-4 mol/kg; or about 1-3 mol/kg; or about 1-2 mol/kg; or about 2-6 mol/kg; or about 2-5 mol/kg; or about 2-4 mol/kg; or about 2-3 mol/kg; or about 3-6 mol/kg; or about 3-5.5 mol/kg; or about 3-5 mol/kg; or about 3-4.5 mol/kg; or about 3-4 mol/kg; or about 4-6 mol/kg; or about 4-5.5 mol/kg; or about 4-5 mol/kg; or about 5-6 mol/kg of the solution. For example only, in some embodiments, the Lewis acid selected from SiBr₄; GeBr₄; SnBr₄; BBr₃; AlBr₃; GaBr₃; InBr₃; TlBr₃; PBr₃; SbBr₃; AsBr₃; CuBr₂; ZnBr₂; TiBr₃; TiBr₄; VBr₄; CrBr₂; MnBr₂; FeBr₂; FeBr₃; CoBr₂; NiBr₂; LaBr₃; and Sc(OTf)₃ is in a concentration in a range of about 0.1-6 mol/kg of the solution.

In some embodiments of the foregoing aspects and embodiments, hydrobromic acid (HBr) can improve the yield and/or the selectivity of the PBH during the hydrolysis of the DBP using Lewis acid. In some embodiments of the foregoing aspects and embodiments, addition of the HBr can improve the recovery of the HBr from the solution. Accordingly, in some embodiments of the foregoing aspects and embodiments, there are provided methods to form PBH, comprising: hydrolyzing DBP to PBH and propanal in an aqueous solution comprising Lewis acid and HBr. In some embodiments of the foregoing aspects and embodiments, there are provided methods to form BE, comprising: hydrolyzing DBE to BE and bromoacetaldehyde in an aqueous solution comprising Lewis acid and HBr. The HBr may be added to the hydrolysis reaction/reactor (the “other HBr” as explained herein) in addition to the co-produced HBr that is retained in the reactor. The hydrolysis reaction may be carried out in the presence of between about 1-20 wt % HBr; or between about 2-20 wt % HBr; or between about 5-20 wt % HBr; or between about 8-20 wt % HBr; or between about 10-20 wt % HBr; or between about 15-20 wt % HBr; or between about 10-15 wt % HBr; or between about 3-15 wt % HBr; or between about 4-10 wt % HBr.

In some embodiments, there are provided methods to form PBH or BE, comprising: hydrolyzing the DBP to the PBH and propanal or the DBE to the BE and bromoacetaldehyde in an aqueous solution comprising Lewis acid in concentration of between about 0.1-6 mol/kg of the solution and HBr in concentration of between about 1-20 wt %. In some embodiments, there are provided methods to form PBH or BE, comprising: hydrolyzing the DBP to the PBH and propanal or the DBE to the BE and bromoacetaldehyde in an aqueous solution comprising ZnBr₂ in concentration of between about 0.1-6 mol/kg of the solution and HBr in concentration of between about 1-20 wt %. In some embodiments, there are provided methods to form PBH or BE, comprising: hydrolyzing the DBP to the PBH and propanal or the DBE to the BE and bromoacetaldehyde in an aqueous solution comprising SnBr₄ in concentration of between about 0.1-6 mol/kg of the solution and HBr in concentration of between about 1-20 wt %.

In some embodiments, there are provided methods to form PBH or BE, comprising: hydrolyzing the DBP to the PBH and propanal or the DBE to the BE and bromoacetaldehyde in an aqueous solution comprising Lewis acid in concentration of about 0.1-6 mol/kg; or about 0.1-5.5 mol/kg; or about 0.1-5 mol/kg; or about 0.1-4.5 mol/kg; or about 0.1-4 mol/kg; or about 0.1-3.5 mol/kg; or about 0.1-3 mol/kg; or about 0.1-2.5 mol/kg; or about 0.1-2 mol/kg; or about 0.1-1.5 mol/kg; or about 0.1-1 mol/kg; or about 0.1-0.5 mol/kg; or about 0.5-6 mol/kg; or about 0.5-5 mol/kg; or about 0.5-4 mol/kg; or about 0.5-3 mol/kg; or about 0.5-2 mol/kg; or about 0.5-1 mol/kg; or about 1-6 mol/kg; or about 1-5 mol/kg; or about 1-4 mol/kg; or about 1-3 mol/kg; or about 1-2 mol/kg; or about 2-6 mol/kg; or about 2-5 mol/kg; or about 2-4 mol/kg; or about 2-3 mol/kg; or about 3-6 mol/kg; or about 3-5.5 mol/kg; or about 3-5 mol/kg; or about 3-4.5 mol/kg; or about 3-4 mol/kg; or about 4-6 mol/kg; or about 4-5.5 mol/kg; or about 4-5 mol/kg; or about 5-6 mol/kg, of the solution; and HBr in concentration of between about 1-20 wt %; or between about 2-20 wt %; or between about 5-20 wt %; or between about 8-20 wt %; or between about 10-20 wt %; or between about 15-20 wt %; or between about 10-15 wt %; or between about 3-15 wt %; or between about 4-10 wt % HBr. Any combination of the concentration of the Lewis acid and the HBr can be employed and all are within the scope of the invention.

In some embodiments of the foregoing aspect and embodiments, the hydrolysis reaction of the DBP to make the PBH and propanal or the DBE to the BE and bromoacetaldehyde using the Lewis acid is carried out in conditions that allow for the recovery of the HBr. For example, the recovered HBr can be recycled to facilitate other chemical processes such as oxybromination of CuBr to CuBr₂, which can then be used for further conversion of the propylene (described in detail herein). To recover the co-produced HBr in an economic manner it may be recoverable in a concentrated form such that the produced HBr can be removed through vaporization without significant cost (as the HBr may be recovered from the vapor leaving the reactor). Because the HBr and water may form a high boiling azeotrope, it may be valuable to find a reactor composition whereby the vapor phase concentration of the HBr is near or above this threshold. This may be accomplished by two variables: elevated HBr concentration and/or elevated bromide salt concentration. Increasing HBr concentration in the aqueous phase can increase the HBr concentration in the vapor phase. As described above, the HBr may be added to the hydrolysis reaction/reactor in addition to the co-produced HBr that is retained in the reactor.

The bromide salts (or salt), as noted above, may bind to free water molecules so that the vapor phase HBr concentration may increase. The high bromide salt concentration may be achieved by using high Lewis acid concentration when the Lewis acid is a bromide salt (e.g. zinc bromide, tin bromide, aluminum bromide etc.). In some embodiments, one or more bromide salt(s) may be added to the hydrolysis reaction. The “bromide salt” as used herein includes alkali metal bromide or alkaline earth metal bromide. Examples include, without limitation, sodium bromide, lithium bromide, potassium bromide, calcium bromide, magnesium bromide, barium bromide, strontium bromide, etc.

In some embodiments of the foregoing aspect and embodiments, there are provided methods to form PBH or BE, comprising: hydrolyzing DBP to PBH and propanal or DBE to BE and bromoacetaldehyde in an aqueous solution comprising Lewis acid and one or more bromide salts. In some embodiments, the aqueous solution comprising Lewis acid and one or more bromide salts, further comprises the HBr.

In some embodiments, there are provided methods to form PBH or BE, comprising: hydrolyzing DBP to PBH and propanal or DBE to BE and bromoacetaldehyde in an aqueous solution comprising Lewis acid in concentration of between about 0.1-6 mol/kg of the solution; and one or more bromide salts in concentration of between about 1-30 wt %. In some embodiments, there are provided methods to form PBH or BE, comprising: hydrolyzing DBP to PBH and propanal or DBE to BE and bromoacetaldehyde in an aqueous solution comprising Lewis acid in concentration of between about 0.1-6 mol/kg of the solution; HBr in concentration of between about 1-20 wt % or 2-20 wt %; and one or more bromide salts in concentration of between about 1-30 wt %.

In some embodiments, there are provided methods to form PBH or BE, comprising: hydrolyzing DBP to PBH and propanal or DBE to BE and bromoacetaldehyde in an aqueous solution comprising ZnBr₂ in concentration of between about 0.1-6 mol/kg of the solution; and one or more bromide salts in concentration of between about 1-30 wt %. In some embodiments, there are provided methods to form PBH or BE, comprising: hydrolyzing DBP to PBH and propanal or DBE to BE and bromoacetaldehyde in an aqueous solution comprising ZnBr₂ in concentration of between about 0.1-6 mol/kg of the solution; HBr in concentration of between about 1-20 wt % or 2-20 wt %; and one or more bromide salts in concentration of between about 1-30 wt %.

In some embodiments, there are provided methods to form PBH or BE, comprising: hydrolyzing DBP to PBH and propanal or DBE to BE and bromoacetaldehyde in an aqueous solution comprising SnBr₄ in concentration of between about 0.1-6 mol/kg of the solution; and one or more bromide salts in concentration of between about 1-30 wt %. In some embodiments, there are provided methods to form PBH or BE, comprising: hydrolyzing DBP to PBH and propanal or DBE to BE and bromoacetaldehyde in an aqueous solution comprising SnBr₄ in concentration of between about 0.1-6 mol/kg of the solution; HBr in concentration of between about 1-20 wt % or 2-20 wt %; and one or more bromide salts in concentration of between about 1-30 wt %.

In some embodiments, there are provided methods to form PBH or BE, comprising: hydrolyzing DBP to PBH and propanal or DBE to BE and bromoacetaldehyde in an aqueous solution comprising Lewis acid in concentration of between about 0.1-6 mol/kg of the solution; and an alkaline earth metal bromide e.g. calcium bromide or alkali metal bromide, e.g. sodium bromide in concentration of between about 1-30 wt %. In some embodiments, there are provided methods to form PBH or BE, comprising: hydrolyzing DBP to PBH and propanal or DBE to BE and bromoacetaldehyde in an aqueous solution comprising Lewis acid in concentration of between about 0.1-6 mol/kg of the solution; HBr in concentration of between about 1-20 wt %; and an alkaline earth metal bromide e.g. calcium bromide or alkali metal bromide, e.g. sodium bromide in concentration of between about 1-30 wt %.

In some embodiments, there are provided methods to form PBH or BE, comprising: hydrolyzing DBP to PBH and propanal or DBE to BE and bromoacetaldehyde in an aqueous solution comprising ZnBr₂ in concentration of between about 0.1-6 mol/kg of the solution; and an alkaline earth metal bromide e.g. calcium bromide or alkali metal bromide, e.g. sodium bromide in concentration of between about 1-30 wt %. In some embodiments, there are provided methods to form PBH or BE, comprising: hydrolyzing DBP to PBH and propanal or DBE to BE and bromoacetaldehyde in an aqueous solution comprising ZnBr₂ in concentration of between about 0.1-6 mol/kg of the solution; HBr in concentration of between about 1-20 wt %; and an alkaline earth metal bromide e.g. calcium bromide or alkali metal bromide, e.g. sodium bromide in concentration of between about 1-30 wt %.

In some embodiments, there are provided methods to form PBH or BE, comprising: hydrolyzing DBP to PBH and propanal or DBE to BE and bromoacetaldehyde in an aqueous solution comprising SnBr₄ in concentration of between about 0.1-6 mol/kg of the solution; and an alkaline earth metal bromide e.g. calcium bromide or alkali metal bromide, e.g. sodium bromide in concentration of between about 1-30 wt %. In some embodiments, there are provided methods to form PBH or BE, comprising: hydrolyzing DBP to PBH and propanal or DBE to BE and bromoacetaldehyde in an aqueous solution comprising SnBr₄ in concentration of between about 0.1-6 mol/kg of the solution; HBr in concentration of between about 1-20 wt %; and an alkaline earth metal bromide e.g. calcium bromide or alkali metal bromide, e.g. sodium bromide in concentration of between about 1-30 wt %.

In some embodiments, the one or more bromide salts (for example only, sodium bromide and/or calcium bromide) in the hydrolysis reaction include between about 1-30 wt % salt; or between 1-25 wt % salt; or between 1-20 wt % salt; or between 1-10 wt % salt; or between 5-30 wt % salt; or between 5-20 wt % salt; or between 5-10 wt % salt; or between about 8-30 wt % salt; or between about 8-25 wt % salt; or between about 8-20 wt % salt; or between about 8-15 wt % salt; or between about 10-30 wt % salt; or between about 10-25 wt % salt; or between about 10-20 wt % salt; or between about 10-15 wt % salt; or between about 15-30 wt % salt; or between about 15-25 wt % salt; or between about 15-20 wt % salt; or between about 20-30 wt % salt; or between about 20-25 wt % salt.

In some embodiments of the foregoing aspect and embodiments, reaction conditions for the hydrolysis reaction comprise temperature between 120-160° C., pressure between 125-350 psig or 0-350 psig, or combination thereof. In some embodiments, the temperature of the hydrolysis reaction/reactor is between 20° C.-200° C. or between 90° C.-160° C.

In some embodiments, the water in the hydrolysis reaction is between 5-50%; or 5-40%; or 5-30%; or 5-20%; or 5-10%; or 50-75%; or 50-70%; or 50-65%; or 50-60% by weight.

In some embodiments, the hydrolysis reaction conditions in the methods to form the PBH and propanal comprise varying the residence time of the hydrolysis solution. The “incubation time” or “residence time” or “mean residence time” as used herein includes the time period for which the hydrolysis solution is left in the reactor at the above noted temperatures before being taken out for the separation of the product. In some embodiments, the residence time for the hydrolysis solution is few seconds or between about 1 sec-1 hour; or 1 sec-5 hours; or 10 min-5 hours or more depending on the temperature of the hydrolysis solution. This residence time may be in combination with other reaction conditions such as, e.g. the temperature ranges and/or bromide concentrations provided herein. In some embodiments, the residence time for the hydrolysis solution is between about 1 sec-3 hour; or between about 1 sec-2.5 hour; or between about 1 sec-2 hour; or between about 1 sec-1.5 hour; or between about 1 sec-1 hour; or 10 min-3 hour; or between about 10 min-2.5 hour; or between about 10 min-2 hour; or between about 10 min-1.5 hour; or between about 10 min-1 hour; or between about 10 min-30 min; or between about 20 min-3 hour; or between about 20 min-2 hour; or between about 20 min-1 hour; or between about 30 min-3 hour; or between about 30 min-2 hour; or between about 30 min-1 hour; or between about 1 hour-2 hour; or between about 1 hour-3 hour; or between about 2 hour-3 hour, to form the PBH and propanal or BE and bromoacetaldehyde (or other bromo derivatives) as noted herein. In some embodiments, the residence time in the hydrolysis reaction/reactor is less than two hours or less than one hour.

In some embodiments of the foregoing aspect and embodiments, the hydrolysis of the DBP to the PBH and propanal or the DBE to the BE and bromoacetaldehyde in the aqueous Lewis acid solution and optionally HBr and/or one or more bromide salts, may be maximized if the aqueous medium can be saturated with the DBP or DBE. In some embodiments, the DBP or DBE may be present in excess amount in order to facilitate efficient hydrolysis. In some embodiments, the DBP or DBE may be as high as 10-95% by volume; or 10-90% by volume; or 10-80% by volume; or 10-70% by volume; or 10-60% by volume; or 10-50% by volume; or 10-40% by volume; or 10-30% by volume; or 10-20% by volume; or 25-95% by volume; or 25-90% by volume; or 25-80% by volume; or 25-70% by volume; or 25-60% by volume; or 25-50% by volume; or 50-95% by volume; or 50-75% by volume; or 75-95% by volume, of the total solution volume.

The above noted DBP amount or the DBE amount can be obtained by using the DBP or the DBE stream from one bromination reaction or from several bromination reactions. Such bromination reactions have been described in detail herein. The above noted amount of the DBP or the DBE can form a second organic phase which may help ensure that a soluble concentration of the DBP or the DBE remains in the aqueous phase. In some embodiments, further derivatization of the PBH into other products (such as, but not limited to, acetone, propanal, bromopropanals, and/or propylene glycol) may be minimized as the PBH may preferentially partition into the DBP phase rather than the aqueous phase. In a continuous operation, the PBH and the propanal may be removed from the reactor in the organic phase with the un-reacted DBP. This last advantage may alleviate the need to separate the PBH from the aqueous solution by other techniques such as distillation.

In some embodiments, the PBH may be extracted from the hydrolysis solution using DBP as an extraction solvent (described in detail herein). By extracting the PBH with the DBP, the PBH can be removed from the bromination reactor by removing the DBP layer that is phase-separated from the aqueous layer. Similarly, In some embodiments, the BE may be extracted from the hydrolysis solution using DBE as an extraction solvent (described in detail herein). By extracting the BE with the DBE, the BE can be removed from the bromination reactor by removing the DBE layer that is phase-separated from the aqueous layer.

In some embodiments of the above noted aspect, the method comprises separating the DBP from the aqueous medium and/or from the PBH and then hydrolyzing the DBP to the PBH and propanal. Similarly, in some embodiments of the above noted aspect, the method comprises separating the DBE from the aqueous medium and/or from the BE and then hydrolyzing the DBE to the BE and bromoacetaldehyde. In such embodiments, a separation step takes place between the bromination and the hydrolysis. It is to be noted that some hydrolysis may take place during separation step itself.

In one aspect, there are provided methods to form PBH, comprising: (i) contacting an anode with an anode electrolyte in an electrochemical cell wherein the anode electrolyte comprises metal bromide with metal ion in a lower oxidation state, metal bromide with metal ion in a higher oxidation state, and saltwater; contacting a cathode with a cathode electrolyte in the electrochemical cell; applying voltage to the anode and the cathode and oxidizing the metal bromide with metal ion in a lower oxidation state to a higher oxidation state at the anode; (ii) withdrawing the anode electrolyte from the electrochemical cell and brominating propylene in the anode electrolyte comprising metal bromide with metal ion in higher oxidation state and the saltwater to result in one or more products comprising PBH and DBP, and the metal bromide with the metal ion in lower oxidation state; (iii) separating the PBH from the aqueous medium; and (iv) treating the aqueous medium comprising the metal bromide with metal ions in the higher oxidation state and the lower oxidation state and the DBP with water to hydrolyze the DBP to the PBH and propanal. In one aspect, there are provided methods to form BE, comprising: (i) contacting an anode with an anode electrolyte in an electrochemical cell wherein the anode electrolyte comprises metal bromide with metal ion in a lower oxidation state, metal bromide with metal ion in a higher oxidation state, and saltwater; contacting a cathode with a cathode electrolyte in the electrochemical cell; applying voltage to the anode and the cathode and oxidizing the metal bromide with metal ion in a lower oxidation state to a higher oxidation state at the anode; (ii) withdrawing the anode electrolyte from the electrochemical cell and brominating ethylene in the anode electrolyte comprising metal bromide with metal ion in higher oxidation state and the saltwater to result in one or more products comprising BE and DBE, and the metal bromide with the metal ion in lower oxidation state; (iii) separating the BE from the aqueous medium; and (iv) treating the aqueous medium comprising the metal bromide with metal ions in the higher oxidation state and the lower oxidation state and the DBE with water to hydrolyze the DBE to the BE and optionally bromoacetaldehyde.

In one aspect, there are provided methods to form PBH, comprising: (i) oxidizing metal bromide with metal ion in a lower oxidation state to a higher oxidation state in presence of an oxidant in an oxybromination reaction; (ii) withdrawing the metal bromide with metal ion in the higher oxidation state from the oxybromination reaction and brominating propylene with the metal bromide with the metal ion in the higher oxidation state in saltwater under reaction conditions to result in one or more products comprising PBH and DBP, and the metal bromide with the metal ion in lower oxidation state; (iii) separating the PBH from the aqueous medium; and (iv) treating the aqueous medium comprising the metal bromide with metal ions in the higher oxidation state and the lower oxidation state and the DBP with water to hydrolyze the DBP to the PBH and propanal. In one aspect, there are provided methods to form BE, comprising: (i) oxidizing metal bromide with metal ion in a lower oxidation state to a higher oxidation state in presence of an oxidant in an oxybromination reaction; (ii) withdrawing the metal bromide with metal ion in the higher oxidation state from the oxybromination reaction and brominating ethylene with the metal bromide with the metal ion in the higher oxidation state in saltwater under reaction conditions to result in one or more products comprising BE and DBE, and the metal bromide with the metal ion in lower oxidation state; (iii) separating the BE from the aqueous medium; and (iv) treating the aqueous medium comprising the metal bromide with metal ions in the higher oxidation state and the lower oxidation state and the DBE with water to hydrolyze the DBE to the BE and bromoacetaldehyde.

In some embodiments of the foregoing aspects and embodiments, the methods further include (v) epoxidizing the PBH with a base to form PO. In some embodiments of the foregoing aspects and embodiments, the methods further include (v) epoxidizing the BE with a base to form the EO.

The PBH and propanal or the BE and bromoacetaldehyde may be separated from the aqueous stream and/or from DBP or DBE, respectively, alone or in combination, using various separation techniques, including, but not limited to, reactive separation, distillation, molecular sieve, membrane, extraction, etc. It is to be understood that some amount of DBP may be converted to the PBH or some amount of DBE may be converted to the BE during the separation step (also called reactive separation).

In one aspect, both the DBP and the PBH are separated from the aqueous stream and the DBP is hydrolyzed to the PBH in the absence of the metal salts used in the bromination of the propylene (e.g. metal bromides used in the bromination of propylene). Similarly, in one aspect, both the DBE and the BE are separated from the aqueous stream and the DBE is hydrolyzed to the BE in the absence of the metal salts used in the bromination of the ethylene (e.g. metal bromides used in the bromination of ethylene).

Accordingly, in one aspect, there are provided methods to form PBH, comprising: (i) contacting an anode with an anode electrolyte in an electrochemical cell wherein the anode electrolyte comprises metal bromide with metal ion in a lower oxidation state, metal bromide with metal ion in a higher oxidation state, and saltwater; contacting a cathode with a cathode electrolyte in the electrochemical cell; applying voltage to the anode and the cathode and oxidizing the metal bromide with metal ion in a lower oxidation state to a higher oxidation state at the anode; (ii) withdrawing the anode electrolyte from the electrochemical cell and brominating propylene in the anode electrolyte comprising metal bromide with metal ion in higher oxidation state to result in one or more products comprising PBH and DBP, and the metal bromide with the metal ion in lower oxidation state; (iii) separating organics comprising the PBH and the DBP from the aqueous medium comprising the metal bromide with metal ions in the higher oxidation state and the lower oxidation state; and (iv) hydrolyzing the DBP (also containing PBH) with water to form the PBH and propanal. In one aspect, there are provided methods to form BE, comprising: (i) contacting an anode with an anode electrolyte in an electrochemical cell wherein the anode electrolyte comprises metal bromide with metal ion in a lower oxidation state, metal bromide with metal ion in a higher oxidation state, and saltwater; contacting a cathode with a cathode electrolyte in the electrochemical cell; applying voltage to the anode and the cathode and oxidizing the metal bromide with metal ion in a lower oxidation state to a higher oxidation state at the anode; (ii) withdrawing the anode electrolyte from the electrochemical cell and brominating ethylene in the anode electrolyte comprising metal bromide with metal ion in higher oxidation state to result in one or more products comprising BE and DBE, and the metal bromide with the metal ion in lower oxidation state; (iii) separating organics comprising the BE and the DBE from the aqueous medium comprising the metal bromide with metal ions in the higher oxidation state and the lower oxidation state; and (iv) hydrolyzing the DBE (also containing BE) with water to form the BE.

In one aspect, there are provided methods to form PBH, comprising: (i) oxidizing metal bromide with metal ion in a lower oxidation state to a higher oxidation state in presence of an oxidant in an oxybromination reaction; (ii) withdrawing the metal bromide with metal ion in the higher oxidation state from the oxybromination reaction and brominating propylene with the metal bromide with the metal ion in the higher oxidation state in saltwater under reaction conditions to result in one or more products comprising PBH and DBP, and the metal bromide with the metal ion in lower oxidation state; (iii) separating organics comprising the PBH and the DBP from the aqueous medium comprising the metal bromide with metal ions in the higher oxidation state and the lower oxidation state; and (iv) hydrolyzing the DBP (also containing PBH) with water to form the PBH and propanal. In one aspect, there are provided methods to form BE, comprising: (i) oxidizing metal bromide with metal ion in a lower oxidation state to a higher oxidation state in presence of an oxidant in an oxybromination reaction; (ii) withdrawing the metal bromide with metal ion in the higher oxidation state from the oxybromination reaction and brominating ethylene with the metal bromide with the metal ion in the higher oxidation state in saltwater under reaction conditions to result in one or more products comprising BE and DBE, and the metal bromide with the metal ion in lower oxidation state; (iii) separating organics comprising the BE and the DBE from the aqueous medium comprising the metal bromide with metal ions in the higher oxidation state and the lower oxidation state; and (iv) hydrolyzing the DBE (also containing BE) with water to form the BE.

In some embodiments of the foregoing aspects, the DBP is separated from the PBH or the DBE is separated from the BE before the hydrolysis step. In some embodiments of the foregoing aspects, the method further includes epoxidizing the PBH with a base to form PO. In some embodiments of the foregoing aspects, the method further includes epoxidizing the BE with a base to form EO. In some embodiments of the foregoing aspects, the method further includes returning the salt from the epoxidation reaction to the electrochemical reaction and/or oxybromination reaction.

In some embodiments, the hydrolysis step forms HBr and the method further comprises recirculating the HBr to the oxybromination step where the metal bromide with the metal ion in the lower oxidation state is converted to the metal bromide with the metal ion in the higher oxidation state in presence of the HBr and oxygen, or hydrogen peroxide, or any other oxidant described herein.

In some embodiments, the bromination reaction may be run in reaction conditions, as described earlier. In such embodiments, both the PBH and the DBP may be separated from the aqueous medium comprising metal bromide as stated above.

In some embodiments, the step of separating the one or more products comprising DBP or DBE from the bromination reaction comprises any separation method known in the art. In some embodiments, the one or more products comprising DBP and optionally the PBH or the one or more products comprising DBE and optionally the BE, may be separated from the bromination reaction as a vapor stream. The separated vapors may be cooled and/or compressed and subjected to the hydrolysis reaction and/or epoxide reaction. Other separation methods include, without limitation, distillation and/or flash distillation using the distillation column or flash distillation drum/column or combinations thereof. The remaining one or more products comprising DBP and optionally the PBH or the one or more products comprising DBE and optionally the BE, in the aqueous medium may be further separated using methods such as, decantation, extraction, or combination thereof. Various examples of the separation methods are described in detail in U.S. patent application Ser. No. 14/446,791, filed Jul. 30, 2014, which is incorporated herein by reference in its entirety.

In one aspect, DBP may be used as an extraction solvent that extracts the DBP and the PBH from the aqueous stream from the bromination reaction/reactor. In one aspect, DBE may be used as an extraction solvent that extracts the DBE and the BE from the aqueous stream from the bromination reaction/reactor. The DBP or the DBE used as the extraction solvent can be the DBP or the DBE from the same process that has been separated and recirculated and/or is the other DBP or the other DBE from another source. The extraction solvent can be any organic solvent that removes the DBP and/or the PBH (or the DBE and/or the BE) from the aqueous metal ion solution. Applicants found that in some embodiments, the use of DBP or the DBE as the extraction solvent may ensure that the hydrolysis reaction, which occurs in an aqueous solution with metal bromides (aspect above) or without metal bromides (another aspect above), can have the maximum rate as the aqueous medium can be saturated with the DBP or the DBE. In some embodiments, the DBP or the DBE may be present in excess amount in order to facilitate efficient hydrolysis. In some embodiments, the mol % of the DBP is equal to or greater than the mol % of the PBH. In some embodiments, the mol % of the DBE is equal to or greater than the mol % of the BE. In some embodiments, the DBP or the DBE may be as high as 10-99.99% by volume; or 10-99% by volume; or 10-95% by volume; or 10-90% by volume; or 10-80% by volume; or 10-70% by volume; or 10-60% by volume; or 10-50% by volume; or 10-40% by volume; or 10-30% by volume; or 10-20% by volume, of the total organic solution volume. There may be several benefits to the use of DBP or the DBE as the extraction solvent.

The DBP or the DBE can form a second organic phase which may help ensure that a soluble concentration of DBP or the DBE remains in the aqueous phase. In some embodiments, further derivatization of the PBH into other products (such as, but not limited to, acetone and/or propanal) or the BE into other products, may be minimized as the PBH may preferentially partition into the DBP phase (or BE may preferentially partition into the DBE phase) rather than the aqueous phase. In a continuous operation, the PBH or the BE may be removed from the reactor in the organic phase with the un-reacted DBP or the un-reacted DBE, respectively. This last advantage may alleviate the need to separate the PBH or the BE from the aqueous solution by other techniques such as distillation. By extracting the PBH with the DBP, the PBH can be removed from the bromination reactor by removing the DBP layer that is phase-separated from the aqueous layer. Similarly, by extracting the BE with the DBE, the BE can be removed from the bromination reactor by removing the DBE layer that is phase-separated from the aqueous layer.

The PBH recovered from these reactors along with the DBP and propanal may be then sent to epoxidation, where the PBH is converted to the PO and the unreacted DBP stream is recirculated to the hydrolysis reaction/reactor. The unreacted propanal may be isolated. In this configuration, any DBP made in the propylene bromination reactor may be balanced by conversion to the PBH in the hydrolysis reactor. The order of operations may be determined by process economics. The epoxidation of the PBH to the PO in the presence of the DBP and propanal has been described herein in detail. Similarly, the BE recovered from these reactors along with the DBE (and other bromo derivatives such as bromoacetaldehyde, if any) may be then sent to epoxidation, where the BE is converted to the EO and the unreacted DBE stream is recirculated to the hydrolysis reaction/reactor. In this configuration, any DBE made in the ethylene bromination reactor may be balanced by conversion to the BE in the hydrolysis reactor. The order of operations may be determined by process economics. The epoxidation of the BE to the EO in the presence of the DBE and optionally bromoacetaldehyde has been described herein in detail.

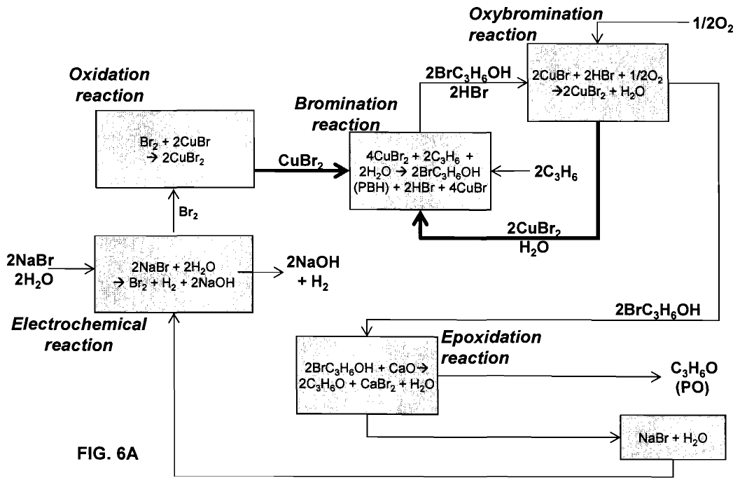

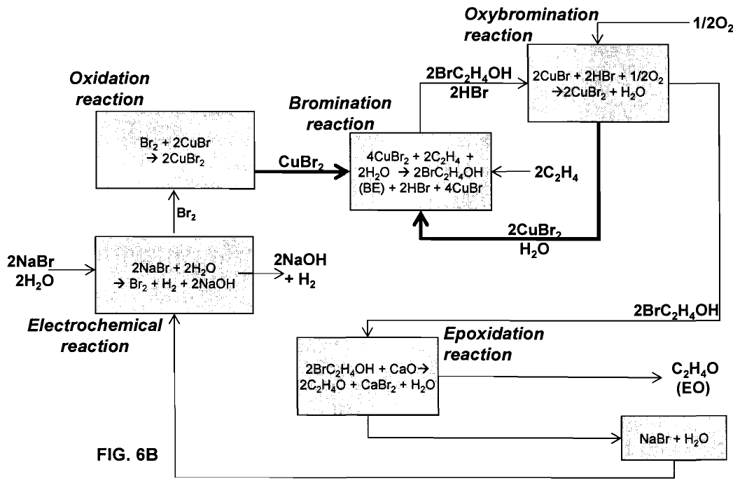

In some embodiments, the DBP or the DBE as the extraction solvent is the DBP or the DBE separated and recirculated from the same process and/or is other DBP or other DBE from other sources. In this embodiment, new or existing sources of bromine to make the DBP via direct bromination of the propylene or the DBE via direct bromination of the ethylene, shown in FIGS. 6A and 6B, are connected to the bromination reactor and/or the hydrolysis reactor for the DBP to be converted to the PBH and ultimately to the PO or for the DBE to be converted to the BE and ultimately to the EO. The HBr formed as a by-product from the conversion to the PBH or the BE would then be captured and reused. The direct bromination of the propylene or the ethylene with the bromine may replace or supplement the electrochemical and/or the oxybromination processes provided herein.

Accordingly, in one aspect, there are provided methods to form PBH, comprising: (i) contacting an anode with an anode electrolyte in an electrochemical cell wherein the anode electrolyte comprises metal bromide with metal ion in a lower oxidation state, metal bromide with metal ion in a higher oxidation state, and saltwater; contacting a cathode with a cathode electrolyte in the electrochemical cell; applying voltage to the anode and the cathode and oxidizing the metal bromide with metal ion in a lower oxidation state to a higher oxidation state at the anode; (ii) withdrawing the anode electrolyte from the electrochemical cell and brominating propylene in the anode electrolyte comprising metal bromide with metal ion in higher oxidation state to result in one or more products comprising PBH and DBP, and the metal bromide with the metal ion in lower oxidation state; (iii) extracting the one or more products comprising PBH and DBP from the aqueous medium by extracting with DBP as an extraction solvent; and (iv) hydrolyzing the DBP with water to form the PBH and propanal and/or epoxidizing the PBH to PO and form unreacted propanal (if epoxidation takes place after the hydrolysis) or unreacted DBP (if epoxidation takes place after the extraction but before the hydrolysis).

Accordingly, in one aspect, there are provided methods to form BE, comprising: (i) contacting an anode with an anode electrolyte in an electrochemical cell wherein the anode electrolyte comprises metal bromide with metal ion in a lower oxidation state, metal bromide with metal ion in a higher oxidation state, and saltwater; contacting a cathode with a cathode electrolyte in the electrochemical cell; applying voltage to the anode and the cathode and oxidizing the metal bromide with metal ion in a lower oxidation state to a higher oxidation state at the anode; (ii) withdrawing the anode electrolyte from the electrochemical cell and brominating ethylene in the anode electrolyte comprising metal bromide with metal ion in higher oxidation state to result in one or more products comprising BE and DBE, and the metal bromide with the metal ion in lower oxidation state; (iii) extracting the one or more products comprising BE and DBE from the aqueous medium by extracting with DBE as an extraction solvent; and (iv) hydrolyzing the DBE with water to form the BE and optionally bromoacetaldehyde and/or epoxidizing the BE to EO and form unreacted bromoacetaldehyde (if epoxidation takes place after the hydrolysis) or unreacted DBE (if epoxidation takes place after the extraction but before the hydrolysis).

In one aspect, there are provided methods to form PBH, comprising: (i) oxidizing metal bromide with metal ion in a lower oxidation state to a higher oxidation state in presence of an oxidant in an oxybromination reaction; (ii) withdrawing the metal bromide with metal ion in the higher oxidation state from the oxybromination reaction and brominating propylene with the metal bromide with the metal ion in the higher oxidation state in saltwater under reaction conditions to result in one or more products comprising PBH and DBP, and the metal bromide with the metal ion in lower oxidation state; (iii) extracting the one or more products comprising PBH and DBP from the aqueous medium by extracting with DBP as an extraction solvent; and (iv) hydrolyzing the DBP with water to form the PBH and propanal and/or epoxidizing the PBH to PO and form unreacted propanal (if epoxidation takes place after the hydrolysis) or unreacted DBP (if epoxidation takes place after the extraction but before the hydrolysis).

In one aspect, there are provided methods to form BE, comprising: (i) oxidizing metal bromide with metal ion in a lower oxidation state to a higher oxidation state in presence of an oxidant in an oxybromination reaction; (ii) withdrawing the metal bromide with metal ion in the higher oxidation state from the oxybromination reaction and brominating ethylene with the metal bromide with the metal ion in the higher oxidation state in saltwater under reaction conditions to result in one or more products comprising BE and DBE, and the metal bromide with the metal ion in lower oxidation state; (iii) extracting the one or more products comprising BE and DBE from the aqueous medium by extracting with DBE as an extraction solvent; and (iv) hydrolyzing the DBE with water to form the BE and optionally bromoacetaldehyde and/or epoxidizing the BE to EO and form unreacted bromoacetaldehyde (if epoxidation takes place after the hydrolysis) or unreacted DBE (if epoxidation takes place after the extraction but before the hydrolysis).

It is to be understood that in all the aspects and embodiments provided herein, the anode electrolyte withdrawn from the electrochemical cell and/or the metal bromide with metal ion in the higher oxidation state withdrawn from the oxybromination reaction, comprise both the metal bromide with the metal ion in the lower oxidation state as well as the metal bromide with the metal ion in the higher oxidation state (e.g. CuBrₓ).

In some embodiments, the method further includes after extraction, transferring aqueous medium comprising the metal bromide with metal ions in the higher oxidation state and the lower oxidation state to the oxybrominating reaction/reactor; to the hydrolysis reaction/reactor; to the bromination reaction/reactor; and/or to the electrochemical reaction/cell.

In some embodiments, the temperature and the residence time in the hydrolysis reaction/reactor may be different from the one in the bromination reaction/reactor. For example, in some embodiments, the hydrolysis reaction may be run at a higher temperature than the bromination reaction. In some embodiments, the temperature in the hydrolysis reaction or the hydrolyzing reactor include, but not limited to, between about 20-200° C.; or between about 20-150° C.; or between about 20-100° C.; or between about 20-50° C.; or between about 50-200° C.; or between about 50-150° C.; or between about 50-100° C.; or between about 100-200° C.; or between about 100-150° C.; or between about 110-150° C.; or between about 120-150° C.; or between about 130-150° C.; or between about 140-150° C.; or between about 90-160° C.; or between about 100-140° C.; or between about 110-140° C.; or between about 120-140° C.; or between about 120-160° C.; or between about 130-140° C.; or between about 100-130° C.; or between about 110-130° C.; or between about 120-130° C.; or between about 100-120° C.; or between about 110-120° C.

In some embodiments, the residence time in the hydrolysis reaction may be longer than that in the bromination reaction. The extraction method may be such that once the one or more products comprising DBP and PBH are extracted from the aqueous medium using the DBP as an extraction solvent (or the products comprising DBE and BE are extracted from the aqueous medium using the DBE as an extraction solvent), the organics are transferred to the hydrolysis reaction and/or the epoxidation reaction; the aqueous stream comprising metal bromide with metal ions in the higher oxidation state and the lower oxidation state is added back to the hydrolysis reaction; and the reaction is run at higher temperature and/or longer residence time so that the DBP or the DBE is hydrolyzed to the PBH and propanal or the BE and bromoacetaldehyde, respectively. It is to be understood that the extracted PBH or the extracted BE from the bromination reactor/reaction may be sent directly to the epoxidation reactor/reaction and/or may be sent to the hydrolyzing reactor/reaction or both (as described herein). In some embodiments, the extracted PBH or the extracted BE from the bromination reactor/reaction is sent directly to the epoxidation reactor/reaction without the intermediate step of the hydrolysis reaction or hydrolyzing reactor. The unreacted DBP or the unreacted DBE from the epoxidation reactor/reaction can be then sent to the hydrolysis reaction or hydrolyzing reactor (DBP or DBE loop as described herein).

In some embodiments of the above noted aspect, the method or system further includes transferring the organic medium comprising PBH, propanal, and DBP (remaining if any, after the hydrolyis) from the hydrolysis reaction/reactor to epoxidation reaction/reactor; and epoxidizing the PBH with a base to form PO in the presence of the DBP and propanal (described in detail further herein below). In some embodiments of the above noted aspect, the method further includes transferring the organic medium comprising BE, bromoacetaldehyde, and DBE (remaining if any, after the hydrolyis) from the hydrolysis reaction/reactor to epoxidation reaction/reactor; and epoxidizing the BE with a base to form EO in the presence of the DBE and bromoacetaldehyde (described in detail further herein below). In some embodiments of the foregoing aspects, the method further includes returning the salt from the epoxidation reaction to the electrochemical reaction. Other bromo derivatives from the propylene bromination reaction or the hydrolysis reaction such as bromopropanal, dibromopropanal, or tribromopropanal may also be present in the organic medium. Similarly, other bromo derivatives from the ethylene bromination reaction or the hydrolysis reaction such as bromoacetaldehyde, dibromoacetaldehyde, or tribromoacetaldehyde may also be present in the organic medium.

In some embodiments of the above noted aspects and embodiments, the methods further comprise extracting the PBH and the propanal formed after the hydrolysis step from the aqueous medium using the DBP as an extraction solvent. In some embodiments, where the DBP is used as an extraction solvent for the PBH, the DBP may be separated from the PBH and the separated DBP may be recirculated to the separation reaction/reactor and/or to the hydrolysis reaction/reactor. In some embodiments of the above noted aspects and embodiments, the methods further comprise extracting the BE formed after the hydrolysis step from the aqueous medium using the DBE as an extraction solvent. In some embodiments, where the DBE is used as an extraction solvent for the BE, the DBE may be separated from the BE and the separated DBE may be recirculated to the separation reaction/reactor and/or to the hydrolysis reaction/reactor.

In some embodiments of the foregoing aspect and embodiments, the one or more products after the reaction of propylene further comprise isopropanol and/or isopropyl bromide. In some embodiments of the foregoing aspect and embodiments, the method further comprises converting the isopropanol and/or the isopropyl bromide back to the propylene, DBP, and/or PBH. In some embodiments, other isopropanol and/or other isopropyl bromide (waste streams from other processes or sources) may be used in this process and are converted to more valuable propylene, DBP, and/or PBH.

The selectivity and the STY of the PBH or the BE formed by the methods and systems provided herein, have been described earlier.

### Electrochemical Reaction/Cell

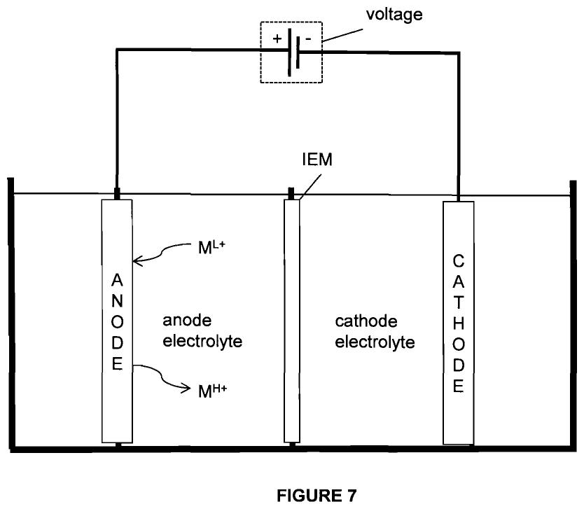

The electrochemical cell or system may be any electrochemical cell that oxidizes metal ions at the anode. Illustrated in FIG. 7 is an electrochemical system having an anode and a cathode separated by an ion exchange membrane. The anode electrolyte contains metal ions in the lower oxidation state (represented as M^{L+}) which are converted by the anode to metal ions in the higher oxidation state (represented as M^{H+}). As used herein “lower oxidation state” represented as L+ in M^{L+} includes the lower oxidation state of the metal. For example, lower oxidation state of the metal ion may be 1+, 2+, 3+, 4+, or 5+. As used herein “higher oxidation state” represented as H+ in M^{H+} includes the higher oxidation state of the metal. For example, higher oxidation state of the metal ion may be 2+, 3+, 4+, 5+, or 6+.

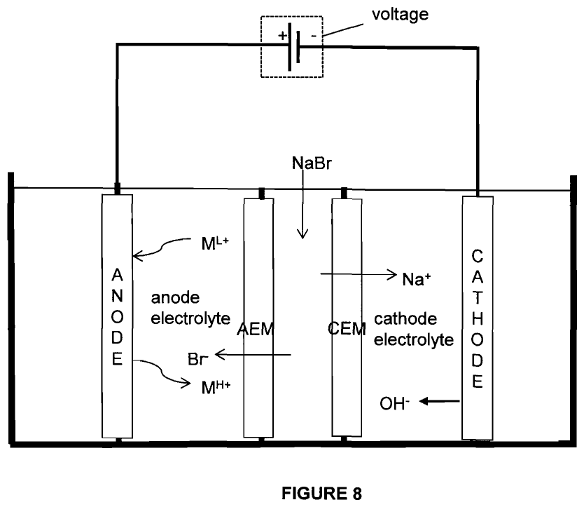

Illustrated in FIG. 8 is an electrochemical system having an anode and a cathode separated by one or more ion exchange membranes, e.g. anion exchange membrane and cation exchange membrane creating a third middle chamber containing a third electrolyte, such as saltwater, e.g. alkali metal bromide or alkali earth metal bromide including but not limited to, sodium bromide; potassium bromide; lithium bromide; magnesium bromide; calcium bromide; strontium bromide, or barium bromide etc. The anode chamber includes the anode and an anode electrolyte in contact with the anode. In some embodiments, the anode electrolyte comprises saltwater and metal bromide. The saltwater comprises alkali metal ions such as, for example only, alkali metal bromide or alkaline earth metal ions such as, for example only, alkaline or alkali earth metal bromide, as described above. The cathode chamber includes the cathode and a cathode electrolyte in contact with the cathode. The cathode electrolyte may also contain saltwater containing alkali metal ions such as, for example only, alkali metal bromide or alkaline earth metal ions such as, for example only, alkaline earth metal bromide, as described above. A combination of the alkali metal bromide and the alkaline earth metal bromide may also be present in anode electrolyte, cathode electrolyte, and/or middle chamber. The cathode electrolyte may also contain alkali metal hydroxide. The metal ion of the metal bromide is oxidized in the anode chamber of the electrochemical cell from the lower oxidation state M^{L+} to the higher oxidation state M^{H+}. The electron(s) generated at the anode are used to drive the reaction at the cathode. The cathode reaction may be any reaction known in the art. The anode chamber and the cathode chamber separated by the ion exchange membrane (IEM) allows the passage of ions, such as, but not limited to, sodium ions in some embodiments to the cathode electrolyte if the anode electrolyte comprises saltwater such as, alkali metal ions (in addition to the metal ions such as metal bromide), such as, sodium bromide. The sodium ions combine with hydroxide ions in the cathode electrolyte to form sodium hydroxide. It is to be understood that while the metal ion of the metal bromide is oxidized from the lower to the higher oxidation state (electrochemical and oxybromination reactions) or reduced from the higher to the lower oxidation state (bromination reaction) in the systems herein, there always is a mixture of the metal bromide with the metal ion in the lower oxidation state and the higher oxidation state in each of the systems. It is also to be understood that the figures presented herein are for illustration purposes only and only illustrate few modes of the systems. The detailed embodiments of each of the systems are described herein and all the combinations of such detailed embodiments can be combined to carry out the invention.

In the electrochemical cells, cathode reaction may be any reaction that does or does not form an alkali in the cathode chamber. Such cathode consumes electrons and carries out any reaction including, but not limited to, the reaction of water to form hydroxide ions and hydrogen gas or reaction of oxygen gas and water to form hydroxide ions or reduction of protons from an acid such as hydrobromic acid to form hydrogen gas or reaction of protons from hydrobromic acid and oxygen gas to form water. In some embodiments, the electrochemical cells may include production of alkali in the cathode chamber of the cell. The alkali generated in the cathode chamber may be used for epoxidation of PBH to PO, epoxidation of BE to EO, or may be used for neutralization of HBr as described herein.

In the embodiments herein, all the methods/systems including electrochemical, bromination, and oxybromination methods/systems comprise metal bromide in saltwater. Various examples of saltwater have been described herein. Further, in the embodiments herein, all the methods/systems including electrochemical, bromination, and oxybromination methods/systems comprise metal bromide in lower oxidation state and higher oxidation state in saltwater. For example only, in the embodiments herein, all the methods/systems including electrochemical, bromination, and oxybromination methods/systems comprise copper bromide in saltwater. In the embodiments herein, the oxidation of the aqueous solution of the metal bromide with the metal ion oxidized from the lower oxidation state to the higher oxidation state in the electrochemical reaction or the oxybromination reaction or the reduction of the aqueous solution of the metal bromide with the metal ion reduced from the higher oxidation state to the lower oxidation state in the bromination reaction is all carried out in the aqueous medium such as saltwater. Examples of saltwater include water comprising alkali metal ions such as alkali metal bromide or alkaline earth metal ions such as alkaline earth metal bromide. Examples include, without limitation, sodium bromide, potassium bromide, lithium bromide, calcium bromide, magnesium bromide etc.

In some embodiments, the temperature of the anode electrolyte in the electrochemical cell/reaction is between 70-100° C., the temperature of the solution in the bromination reactor/reaction is between 40-110° C., the temperature of the solution in the oxybromination reactor/reaction is between 60-90° C., and/or the temperature of the solution in the epoxidation reactor/reaction is between 40-90° C., depending on the configuration of the electrochemical cell/reaction, the bromination reactor/reaction, the oxybromination reactor/reaction, and the epoxidation reactor/reaction. In some embodiments, the lower temperature of the liquid or liquid/gas phase oxybromination provided herein as compared to high temperatures of solid/gas phase oxybromination, may provide economic benefits such as, but not limited to lower capital and operating expenses.

In one aspect, there are provided methods that include

(i) contacting an anode with an anode electrolyte in an electrochemical cell wherein the anode electrolyte comprises metal bromide with metal ion in a lower oxidation state, metal bromide with metal ion in a higher oxidation state, and saltwater; contacting a cathode with a cathode electrolyte in the electrochemical cell; applying a voltage to the anode and the cathode and oxidizing the metal bromide with metal ion in a lower oxidation state to a higher oxidation state at the anode;

(ii) withdrawing the anode electrolyte from the electrochemical cell and brominating propylene with the anode electrolyte comprising the metal bromide with the metal ion in the higher oxidation state in the saltwater under reaction conditions to result in one or more products comprising propylene bromohydrin (PBH) and the metal bromide with the metal ion in the lower oxidation state; or withdrawing the anode electrolyte from the electrochemical cell and brominating ethylene with the anode electrolyte comprising the metal bromide with the metal ion in the higher oxidation state in the saltwater under reaction conditions to result in one or more products comprising bromoethanol (BE) and the metal bromide with the metal ion in the lower oxidation state; and

(iii) epoxidizing the PBH or the BE with a base to form propylene oxide (PO) or ethylene oxide (EO), respectively.

As described herein, in some embodiments, the one or more products comprise DBP and the method further comprises hydrolyzing DBP under one or more reaction conditions to form hydrolysis products comprising PBH and propanal. It is to be understood that one or more combinations of these steps may be carried out together. For example, the step (iii) in series with the step (ii) and the step (i) in series or in parallel with the step (ii) and/or (iii). The steps may be integrated in a single unit or may be more than one separate units running in a plant. Similarly, other combinations may be carried out in a single unit or as separate units in one plant.

In some embodiments, there are provided systems that carry out the methods described herein.

In some embodiments, there are provided systems that include

an electrochemical cell comprising an anode in contact with an anode electrolyte wherein the anode electrolyte comprises metal bromide with metal ion in a lower oxidation state, metal bromide with metal ion in a higher oxidation state, and saltwater; a cathode in contact with a cathode electrolyte; and a voltage source configured to apply a voltage to the anode and the cathode wherein the anode is configured to oxidize the metal bromide with the metal ion from a lower oxidation state to a higher oxidation state;

a bromination reactor operably connected to the electrochemical cell wherein the bromination reactor is configured to receive the metal bromide with the metal ion in the higher oxidation state from the electrochemical cell and brominate propylene or ethylene with the metal bromide with the metal ion in the higher oxidation state under reaction conditions to result in one or more products comprising PBH or one or more products comprising BE, respectively, and the metal bromide solution with the metal ion in the lower oxidation state; and

an epoxide reactor operably connected to the bromination reactor and configured to epoxidize PBH or BE with a base to form PO or EO, respectively.

As described herein, in some embodiments, the one or more products comprise DBP and the system further comprises hydrolyzing reactor operably connected to the bromination reactor and/or the epoxide reactor and configured to hydrolyse DBP (and/or unreacted DBP) under one or more reaction conditions to form hydrolysis products comprising PBH and propanal.

The “metal ion” or “metal” or “metal ion of the metal bromide” as used herein, includes any metal ion capable of being converted from lower oxidation state to higher oxidation state. Examples of metal ions in the corresponding metal bromide include, but not limited to, iron, chromium, copper, tin, silver, cobalt, uranium, lead, mercury, vanadium, bismuth, titanium, ruthenium, osmium, europium, zinc, cadmium, gold, nickel, palladium, platinum, rhodium, iridium, manganese, technetium, rhenium, molybdenum, tungsten, niobium, tantalum, zirconium, hafnium, and combination thereof. In some embodiments, the metal ion in the corresponding metal bromide include, but not limited to, iron, copper, tin, chromium, or combination thereof. In some embodiments, the metal ion in the corresponding metal bromide is copper. In some embodiments, the metal ion in the corresponding metal bromide is tin. In some embodiments, the metal ion in the corresponding metal bromide is iron. In some embodiments, the metal ion in the corresponding metal bromide is chromium. In some embodiments, the metal ion in the corresponding metal bromide is platinum.

The “oxidation state” as used herein, includes degree of oxidation of an atom in a substance. For example, in some embodiments, the oxidation state is the net charge on the ion. Some examples of the reaction of the metal ions at the anode are as shown in Table I below (SHE is standard hydrogen electrode). The theoretical values of the anode potential are also shown. It is to be understood that some variation from these voltages may occur depending on conditions, pH, concentrations of the electrolytes, etc and such variations are well within the scope of the invention.

TABLE I

| | Anode |
|---|---|
| | Potential |
| Anode Reaction | (V vs. SHE) |
| Ag⁺ → Ag²⁺ + e⁻ | −1.98 |
| Co²⁺ → Co³⁺ + e⁻ | −1.82 |
| Pb²⁺ → Pb⁴⁺ + 2e⁻ | −1.69 |
| Ce³⁺ → Ce⁴⁺ + e⁻ | −1.44 |
| 2Cr³⁺ + 7H₂O → Cr₂O₇²⁻ + 14H⁺ + 6e⁻ | −1.33 |
| Ti⁺ → Ti³⁺ + 2e⁻ | −1.25 |
| Hg₂²⁺ → 2Hg²⁺ + 2e⁻ | −0.91 |
| Fe²⁺ → Fe³⁺ + e⁻ | −0.77 |
| V³⁺ + H₂O → VO²⁺ + 2H⁺ + e⁻ | −0.34 |
| U⁴⁺ + 2H₂O → UO²⁺ + 4H⁺ + e⁻ | −0.27 |
| Bi⁺ → Bi³⁺ + 2e⁻ | −0.20 |
| Ti³⁺ + H₂O → TiO²⁺ + 2H⁺ + e⁻ | −0.19 |
| Cu⁺ → Cu²⁺ + e⁻ | −0.16 |
| UO₂⁺ → UO₂²⁺ + e⁻ | −0.16 |
| Sn²⁺ → Sn⁴⁺ + 2e⁻ | −0.15 |
| Ru(NH₃)₆²⁺ → Ru(NH₃)₆³⁺ + e⁻ | −0.10 |
| V²⁺ → V³⁺ + e⁻ | +0.26 |
| Eu²⁺ → Eu³⁺ + e⁻ | +0.35 |
| Cr²⁺ → Cr³⁺ + e⁻ | +0.42 |
| U³⁺ → U⁴⁺ + e⁻ | +0.52 |

The metal bromide may be present as a compound of the metal or an alloy of the metal or combination thereof. In some embodiments, the anion attached to the metal is same as the anion of the electrolyte. For example, for sodium or potassium bromide used as an electrolyte, a metal bromide, such as, but not limited to, iron bromide, copper bromide, tin bromide, chromium bromide etc. is used as the metal compound. In such embodiments, it may be desirable to have sufficient concentration of bromide ions in the electrolyte to dissolve the metal salt but not high enough to cause undesirable ionic speciation. As stated earlier, the concentration of metal bromide effective for high yield and selectivity of PBH is much lower than that required for metal chloride, thereby resulting in improved solubility and workability.

In some embodiments, the metal ions of the metal bromide described herein, may be chosen based on the solubility of the metal in the anode electrolyte and/or cell voltages desired for the metal oxidation from the lower oxidation state to the higher oxidation state.

It is to be understood that the metal bromide with the metal ion in the lower oxidation state and the metal bromide with the metal ion in the higher oxidation state are both present in the anode electrolyte. The anode electrolyte exiting the anode chamber contains higher amount of the metal bromide in the higher oxidation state than the amount of the metal bromide in the higher oxidation state entering the anode chamber. Owing to the oxidation of the metal bromide from the lower oxidation state to the higher oxidation state at the anode, the ratio of the metal bromide in the lower and the higher oxidation state is different in the anode electrolyte entering the anode chamber and exiting the anode chamber. Suitable ratios of the metal ion in the lower and higher oxidation state in the anode electrolyte have been described herein. The mixed metal ion in the lower oxidation state with the metal ion in the higher oxidation state may assist in lower voltages in the electrochemical systems and high yield and selectivity in corresponding bromination reaction with the propylene or ethylene.

In some embodiments, the metal ion in the anode electrolyte is a mixed metal ion. For example, the anode electrolyte containing the copper ion in the lower oxidation state and the copper ion in the higher oxidation state may also contain another metal ion such as, but not limited to, iron. In some embodiments, the presence of a second metal ion in the anode electrolyte may be beneficial in lowering the total energy of the electrochemical reaction in combination with the catalytic reaction.

Some examples of the metal compounds or metal bromide that may be used in the systems and methods of the invention include, but are not limited to, copper (I) bromide, copper (II) bromide, iron (II) bromide, tin (II) bromide, chromium (II) bromide, zinc (II) bromide, etc.

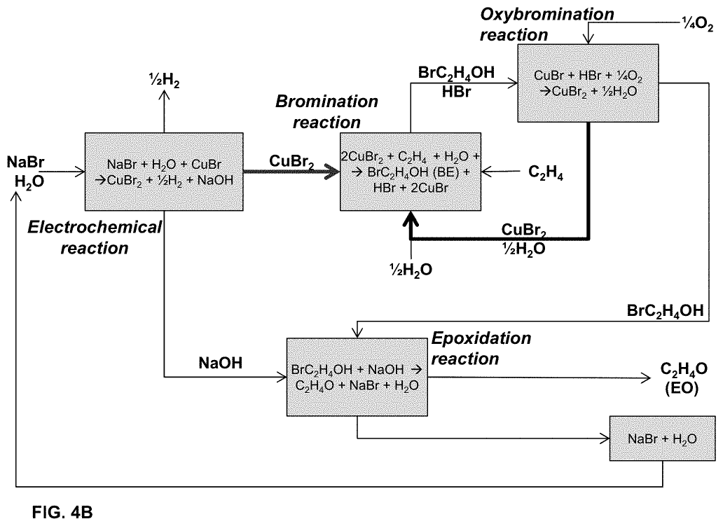

Above noted aspects are as illustrated in FIGS. 3A, 4A, 3B, and 4B. In the electrochemical reaction or cell, a metal bromide, e.g. CuBr is oxidized at the anode to higher oxidation state CuBr₂ in saltwater (illustrated as sodium bromide (NaBr)) when sodium hydroxide (NaOH) and hydrogen gas are formed at the cathode. It is to be understood that the metal bromide illustrated as CuBr and CuBr₂, saltwater illustrated as NaBr, and the cathode reaction to form NaOH and H₂ gas, in all the figures herein, are for illustration purposes only and other variations of the metal bromide, any other salt, and other cathode reactions are well within the scope of the invention some of which have been described in detail herein. The anode electrolyte comprising NaBr and CuBr₂ is withdrawn from the electrochemical cell and is subjected to bromination of propylene in the bromination reaction/reactor when propylene (C₃H₆) is brominated to propylene bromohydrin (PBH), as illustrated in FIG. 3A (or bromination of ethylene (C₂H₄) to bromoethanol (BE) as illustrated in FIG. 3B) and CuBr₂ is reduced to CuBr (metal ion from the higher oxidation state to the lower oxidation state). In the figures illustrated herein, the PBH is illustrated as 1-bromo-2-hydroxy form, however, 2-bromo-1-hydroxy form may also be formed in combination or in isolation. Without being limited by any theory, both isomers may be formed and both may be subsequently converted to the PO. The explicit declaration of one isomer may not be construed as the absence of the other. As described earlier, some amount of the DBP is formed along with the PBH. The DBP can also be hydrolyzed to the PBH as described herein.

In some embodiments of the above noted aspect and embodiments, the one or more products in the bromination reaction further comprise hydrobromic acid (HBr). In some embodiments of the above noted aspect and embodiments, the method further comprises forming sodium hydroxide in the cathode electrolyte and using the sodium hydroxide to neutralize the HBr (shown as neutralization step in FIGS. 3A and 3B).

As illustrated in FIGS. 3A and 3B, two copper bromides may be converted in the electrochemical reaction for every propylene oxide or ethylene oxide that is produced. Since the propylene oxide or ethylene oxide does not contain any bromide, these bromides are ultimately neutralized by 2NaOH molecules (also generated in the electrochemical reaction). In the above method, the OpEx savings compared to a chlor-alkali process (commercial process that electrochemically produces chlorine gas which is then used for chlorination reaction) may be derived from the lower operating voltage of the cell. For example only, compared to a chlor-alkali unit operating at 3V (to generate Cl₂ for chlorination), the electrochemical cell in FIGS. 3A and 3B may effectively be operating at about 2.2-2.8V.

During the bromination reaction, hydrobromic acid is formed (HBr) which is neutralized with NaOH formed at the cathode in the neutralization reaction/reactor. Another mole of NaOH from the cathode electrolyte may be used to epoxidize PBH to propylene oxide (PO) or to epoxidize BE to ethylene oxide (EO) in the epoxidation reaction/reactor. After the bromination reaction, the one or more products comprising PBH from the propylene or one or more products comprising BE from the ethylene may be separated from the aqueous medium (water containing metal bromide and salts and optionally HBr) using various separation techniques described further herein below. The separated one or more products may or may not be subjected to purification before the PBH is epoxidized to PO or before BE is epoxidized to EO in the epoxidation reaction/reactor. As described earlier, due to the boiling point difference between the brominated side products and PO, the separation techniques such as distillation are quite effective in purifying PO. Some or all of the water comprising metal bromides and salts, e.g. NaBr may be recirculated back from the epoxide reaction/reactor to the electrochemical cell for further oxidation of the metal ions at the anode (as shown in the figures).

In some embodiments of the above noted aspect and embodiments, the method further comprises forming sodium hydroxide in the cathode electrolyte and using the sodium hydroxide as the base to form the propylene oxide or ethylene oxide in the epoxidation reaction/reactor and/or using the sodium hydroxide to neutralize the HBr in the neutralization reaction/reactor.

In some embodiments of the above noted system, the system further comprises means for transferring NaOH formed in the cathode chamber of the electrochemical cell to the neutralizing chamber for neutralizing HBr formed in the bromination reactor and/or means for transferring NaOH formed in the cathode chamber of the electrochemical cell to the epoxidation reactor for the epoxidation of PBH to PO or BE to EO. Such means include any means for transferring liquids including, but not limited to, conduits, tanks, pipes, and the like.

All the electrochemical and reactor systems and methods described herein can be carried out in more than 5 wt % water or more than 6 wt % water or aqueous alkali metal bromide. The aqueous alkali metal bromide has been described herein.

The electrochemical cells in the methods and systems provided herein are membrane electrolyzers. The electrochemical cell may be a single cell or may be a stack of cells connected in series or in parallel. The electrochemical cell may be a stack of 5 or 6 or 50 or 100 or more electrolyzers connected in series or in parallel. Each cell comprises an anode, a cathode, and an ion exchange membrane.

In some embodiments, the electrolyzers provided herein are monopolar electrolyzers. In the monopolar electrolyzers, the electrodes may be connected in parallel where all anodes and all cathodes are connected in parallel. In such monopolar electrolyzers, the operation takes place at high amperage and low voltage. In some embodiments, the electrolyzers provided herein are bipolar electrolyzers. In the bipolar electrolyzers, the electrodes may be connected in series where all anodes and all cathodes are connected in series. In such bipolar electrolyzers, the operation takes place at low amperage and high voltage. In some embodiments, the electrolyzers are a combination of monopolar and bipolar electrolyzers and may be called hybrid electrolyzers.

In some embodiments of the bipolar electrolyzers as described above, the cells are stacked serially constituting the overall electrolyzer and are electrically connected in two ways. In bipolar electrolyzers, a single plate, called bipolar plate, may serve as base plate for both the cathode and anode. The electrolyte solution may be hydraulically connected through common manifolds and collectors internal to the cell stack. The stack may be compressed externally to seal all frames and plates against each other which are typically referred to as a filter press design. In some embodiments, the bipolar electrolyzer may also be designed as a series of cells, individually sealed, and electrically connected through back-to-back contact, typically known as a single element design. The single element design may also be connected in parallel in which case it would be a monopolar electrolyzer.

In some embodiments, the cell size may be denoted by the active area dimensions. In some embodiments, the active area of the electrolyzers used herein may range from 0.5-1.5 meters tall and 0.4-3 meters wide. The individual compartment thicknesses may range from 0.5 mm-50 mm.

The electrolyzers used in the methods and systems provided herein, are made from corrosion resistant materials. Variety of materials was tested in metal solutions such as copper and at varying temperatures, for corrosion testing. The materials include, but not limited to, polyvinylidene fluoride, viton, polyether ether ketone, fluorinated ethylene propylene, fiber-reinforced plastic, halar, ultem (PEI), perfluoroalkoxy, tefzel, tyvar, fibre-reinforced plastic-coated with derakane 441-400 resin, graphite, akot, tantalum, hastelloy C2000, titanium Gr.7, titanium Gr.2, or combinations thereof. In some embodiments, these materials can be used for making the electrochemical cells and/or it components including, but not limited to, tank materials, piping, heat exchangers, pumps, reactors, cell housings, cell frames, electrodes, instrumentation, valves, and all other balance of plant materials. In some embodiments, the material used for making the electrochemical cell and its components include, but not limited to, titanium Gr.2.

In some embodiments, the anode may contain a corrosion stable, electrically conductive base support. Such as, but not limited to, amorphous carbon, such as carbon black, fluorinated carbons like the specifically fluorinated carbons described in U.S. Pat. No. 4,908,198 and available under the trademark SFC™ carbons. Other examples of electrically conductive base materials include, but not limited to, sub-stoichiometric titanium oxides, such as, Magneli phase sub-stoichiometric titanium oxides having the formula TiOₓ wherein x ranges from about 1.67 to about 1.9. Some examples of titanium sub-oxides include, without limitation, titanium oxide Ti₄O₇. The electrically conductive base materials also include, without limitation, metal titanates such as MₓTi_{y}O_{z} such as MₓTi₄O₇, etc. In some embodiments, carbon based materials provide a mechanical support or as blending materials to enhance electrical conductivity but may not be used as catalyst support to prevent corrosion.

In some embodiments, the anode is not coated with an electrocatalyst. In some embodiments, the anode is made of an electro conductive base metal such as titanium coated with or without electrocatalysts. Some examples of electrically conductive base materials include, but not limited to, sub-stoichiometric titanium oxides, such as, Magneli phase sub-stoichiometric titanium oxides having the formula TiOₓ wherein x ranges from about 1.67 to about 1.9. Some examples of titanium sub-oxides include, without limitation, titanium oxide Ti₄O₇. The electrically conductive base materials also include, without limitation, metal titanates such as MₓTi_{y}O_{z} such as MₓTi₄O₇, etc. Examples of electrocatalysts have been described herein and include, but not limited to, highly dispersed metals or alloys of the platinum group metals, such as platinum, palladium, ruthenium, rhodium, iridium, or their combinations such as platinum-rhodium, platinum-ruthenium, titanium mesh coated with PtIr mixed metal oxide or titanium coated with galvanized platinum; electrocatalytic metal oxides, such as, but not limited to, IrO₂; gold, tantalum, carbon, graphite, organometallic macrocyclic compounds, and other electrocatalysts well known in the art. The electrodes may be coated with electrocatalysts using processes well known in the art.

In some embodiments, the electrodes described herein, relate to porous homogeneous composite structures as well as heterogeneous, layered type composite structures wherein each layer may have a distinct physical and compositional make-up, e.g. porosity and electroconductive base to prevent flooding, and loss of the three phase interface, and resulting electrode performance.

In some embodiments, the electrodes provided herein may include anodes and cathodes having porous polymeric layers on or adjacent to the anolyte or catholyte solution side of the electrode which may assist in decreasing penetration and electrode fouling. Stable polymeric resins or films may be included in a composite electrode layer adjacent to the anolyte comprising resins formed from non-ionic polymers, such as polystyrene, polyvinyl chloride, polysulfone, etc., or ionic-type charged polymers like those formed from polystyrenesulfonic acid, sulfonated copolymers of styrene and vinylbenzene, carboxylated polymer derivatives, sulfonated or carboxylated polymers having partially or totally fluorinated hydrocarbon chains and aminated polymers like polyvinylpyridine. Stable microporous polymer films may also be included on the dry side to inhibit electrolyte penetration. In some embodiments, the gas-diffusion cathodes includes such cathodes known in the art that are coated with high surface area coatings of precious metals such as gold and/or silver, precious metal alloys, nickel, and the like.

Any of the cathodes provided herein can be used in combination with any of the anodes described above. In some embodiments, the cathode used in the electrochemical systems of the invention, is a hydrogen gas producing cathode.

Following are the reactions that take place at the cathode and the anode:

H₂O+e→½H₂+OH⁻ (cathode)

M^{L+}→M^{H+}+xe⁻ (anode where x=1-3)

For example, Fe²⁺→Fe³⁺+e⁻ (anode)

Cr²⁺→Cr³⁺+e⁻ (anode)

Sn²⁺→Sn⁴⁺+2e⁻ (anode)

Cu⁺→Cu²⁺+e⁻ (anode)

The hydrogen gas formed at the cathode may be vented out or captured and stored for commercial purposes. The M^{H+} formed at the anode combines with bromide ions to form metal bromide in the higher oxidation state such as, but not limited to, FeBr₃, CrBr₃, SnBr₄, or CuBr₂ etc. The hydroxide ion formed at the cathode combines with sodium ions to form sodium hydroxide. In some embodiments, the cathode used in the electrochemical systems of the invention, is a hydrogen gas producing cathode that does not form an alkali. Following are the reactions that take place at the cathode and the anode:

2H⁺+2e⁻→H₂ (cathode)

M^{L+}→M^{H+}+xe⁻ (anode where x=1-3)

For example, Fe²⁺→Fe³⁺+e⁻ (anode)

Cr²⁺→Cr³⁺+e⁻ (anode)

Sn²⁺→Sn⁴⁺+2e⁻ (anode)

Cu⁺→Cu²⁺+e⁻ (anode)

The hydrogen gas may be vented out or captured and stored for commercial purposes. The M^{H+} formed at the anode combines with bromide ions to form metal bromide in the higher oxidation state such as, but not limited to, FeBr₃, CrBr₃, SnBr₄, or CuBr₂ etc.

In some embodiments, the cathode in the electrochemical systems of the invention may be a gas-diffusion cathode. In some embodiments, the cathode in the electrochemical systems of the invention may be a gas-diffusion cathode forming an alkali at the cathode. As used herein, the “gas-diffusion cathode,” or “gas-diffusion electrode,” or other equivalents thereof include any electrode capable of reacting a gas to form ionic species. In some embodiments, the gas-diffusion cathode, as used herein, is an oxygen depolarized cathode (ODC). Such gas-diffusion cathode may be called gas-diffusion electrode, oxygen consuming cathode, oxygen reducing cathode, oxygen breathing cathode, oxygen depolarized cathode, and the like.

Following are the reactions that may take place at the anode and the cathode.

H₂O+½O₂+2e⁻→2OH⁻ (cathode)

M^{L+}→M^{H+}+xe⁻ (anode where x=1-3)

For example, 2Fe²⁺→2Fe³⁺+2e⁻ (anode)

2Cr²⁺→2Cr³⁺+2e⁻ (anode)

Sn²⁺→Sn⁴⁺+2e⁻ (anode)

2Cu⁺→2Cu²⁺+2e⁻ (anode)

The M^{H+} formed at the anode combines with bromide ions to form metal bromide MBrₙ such as, but not limited to, FeBr₃, CrBr₃, SnBr₄, or CuBr₂ etc. The hydroxide ion formed at the cathode reacts with sodium ions to form sodium hydroxide. The oxygen at the cathode may be atmospheric air or any commercial available source of oxygen.

The methods and systems containing the gas-diffusion cathode or the ODC, as described herein may result in voltage savings as compared to methods and systems that include the hydrogen gas producing cathode. The voltage savings in-turn may result in less electricity consumption and less carbon dioxide emission for electricity generation.

While the methods and systems containing the gas-diffusion cathode or the ODC result in voltage savings as compared to methods and systems containing the hydrogen gas producing cathode, both the systems i.e. systems containing the ODC and the systems containing hydrogen gas producing cathode of the invention, show significant voltage savings as compared to chlor-alkali system conventionally known in the art. The voltage savings in-turn may result in less electricity consumption and less carbon dioxide emission for electricity generation. In some embodiments, the electrochemical system of the invention (2 or 3-compartment cells with hydrogen gas producing cathode or ODC) has a theoretical voltage savings of more than 0.5V, or more than 1V, or more than 1.5V, or between 0.5-3V, as compared to chlor-alkali process. In some embodiments, this voltage saving is achieved with a cathode electrolyte pH of between 7-15, or between 7-14, or between 6-12, or between 7-12, or between 7-10.

In some embodiments, the cathode in the electrochemical systems of the invention may be a gas-diffusion cathode that reacts HBr and oxygen gas to form water.

Following are the reactions that may take place at the anode and the cathode.

2H⁺+½O₂+2e⁻→H₂O (cathode)

M^{L+}→M^{H+}+xe⁻ (anode where x=1-3)

For example, 2Fe²⁺→2Fe³⁺+2e⁻ (anode)

2Cr²⁺→2Cr³⁺+2e⁻ (anode)

Sn²⁺→Sn⁴⁺+2e⁻ (anode)

2Cu⁺→2Cu²⁺+2e⁻ (anode)

The M^{H+} formed at the anode combines with bromide ions to form metal bromide MBrₙ such as, but not limited to, FeBr₃, CrBr₃, SnBr₄, or CuBr₂ etc. The oxygen at the cathode may be atmospheric air or any commercial available source of oxygen.

The cathode electrolyte containing the alkali maybe withdrawn from the cathode chamber. The purity of the alkali formed in the methods and systems may vary depending on the end use requirements. For example, methods and systems provided herein that use an electrochemical cell equipped with membranes may form a membrane quality alkali which may be substantially free of impurities. In some embodiments, a less pure alkali may also be formed by avoiding the use of membranes. In some embodiments, the alkali may be separated from the cathode electrolyte using techniques known in the art, including but not limited to, diffusion dialysis. In some embodiments, the alkali formed in the cathode electrolyte is more than 2% w/w or more than 5% w/w or between 5-50% w/w.

In some embodiments, the cathode electrolyte and the anode electrolyte are separated in part or in full by an ion exchange membrane. In some embodiments, the ion exchange membrane is an anion exchange membrane or a cation exchange membrane. In some embodiments, the cation exchange membranes in the electrochemical cell, as disclosed herein, are conventional and are available from, for example, Asahi Kasei of Tokyo, Japan; or from Membrane International of Glen Rock, N.J., or DuPont, in the USA. Examples of CEM include, but are not limited to, N2030WX (Dupont), F8020/F8080 (Flemion), and F6801 (Aciplex). CEMs that are desirable in the methods and systems of the invention have minimal resistance loss, greater than 90% selectivity, and high stability in concentrated caustic. AEMs, in the methods and systems of the invention are exposed to concentrated metallic salt anolytes and saturated brine stream. It is desirable for the AEM to allow passage of salt ion such as bromide ion to the anolyte but reject the metallic ion species from the anolyte.

In some embodiments, the AEM used in the methods and systems provided herein, is also substantially resistant to the organic compounds such that AEM does not interact with the organics and/or the AEM does not react or absorb metal ions. In some embodiments, this can be achieved, for example only, by using a polymer that does not contain a free radical or anion available for reaction with organics or with metal ions. For example only, a fully quarternized amine containing polymer may be used as an AEM.

Examples of cationic exchange membranes include, but not limited to, cationic membrane consisting of a perfluorinated polymer containing anionic groups, for example sulphonic and/or carboxylic groups. However, it may be appreciated that in some embodiments, depending on the need to restrict or allow migration of a specific cation or an anion species between the electrolytes, a cation exchange membrane that is more restrictive and thus allows migration of one species of cations while restricting the migration of another species of cations may be used as, e.g., a cation exchange membrane that allows migration of sodium ions into the cathode electrolyte from the anode electrolyte while restricting migration of other ions out of the catholyte or from the anode electrolyte into the cathode electrolyte, may be used. Similarly, in some embodiments, depending on the need to restrict or allow migration of a specific anion species between the electrolytes, an anion exchange membrane that is more restrictive and thus allows migration of one species of anions while restricting the migration of another species of anions may be used as, e.g., an anion exchange membrane that allows migration of bromide ions into the anode electrolyte while restricting migration of other ions out of the anolyte or from the cathode electrolyte into the anode electrolyte, may be used. Such restrictive cation exchange membranes and anion exchange membranes are commercially available and can be selected by one ordinarily skilled in the art.

In some embodiments, the membranes may be selected such that they can function in an acidic and/or basic electrolytic solution as appropriate. Other desirable characteristics of the membranes include high ion selectivity, low ionic resistance, high burst strength, and high stability in an acidic electrolytic solution in a temperature range of room temperature to 150° C. or higher, or a alkaline solution in similar temperature range may be used. In some embodiments, it is desirable that the ion exchange membrane prevents the transport of the metal ion from the anolyte to the catholyte. In some embodiments, a membrane that is stable in the range of 0° C. to 150° C.; 0° C. to 100° C.; 0° C. to 90° C.; or 0° C. to 80° C.; or 0° C. to 70° C.; or 0° C. to 60° C.; or 0° C. to 50° C.; or 0° C. to 40° C., or 0° C. to 30° C., or 0° C. to 20° C., or 0° C. to 10° C., or higher may be used. For other embodiments, it may be useful to utilize an ion-specific ion exchange membranes that allows migration of one type of cation but not another; or migration of one type of anion and not another, to achieve a desired product or products in an electrolyte. In some embodiments, the membrane may be stable and functional for a desirable length of time in the system, e.g., several days, weeks or months or years at temperatures in the range of 0° C. to 90° C. In some embodiments, for example, the membranes may be stable and functional for at least 1 day, at least 5 days, 10 days, 15 days, 20 days, 100 days, 1000 days, 5-10 years, or more in electrolyte temperatures at 100° C., 90° C., 80° C., 70° C., 60° C., 50° C., 40° C., 30° C., 20° C., 10° C., 5° C. and more or less.

The ohmic resistance of the membranes may affect the voltage drop across the anode and cathode, e.g., as the ohmic resistance of the membranes increase, the voltage across the anode and cathode may increase, and vice versa. Membranes that can be used include, but are not limited to, membranes with relatively low ohmic resistance and relatively high ionic mobility; and membranes with relatively high hydration characteristics that increase with temperatures, and thus decreasing the ohmic resistance. By selecting membranes with lower ohmic resistance known in the art, the voltage drop across the anode and the cathode at a specified temperature can be lowered.

In some embodiments, the aqueous electrolyte including the catholyte or the cathode electrolyte and/or the anolyte or the anode electrolyte, or the third electrolyte disposed between AEM and CEM, in the systems and methods provided herein include, but not limited to, saltwater or fresh water. The saltwater has been described herein. In some embodiments, the depleted saltwater withdrawn from the electrochemical cells is replenished with salt (i.e. alkali metal bromide) and re-circulated back in the electrochemical cell. In some embodiments, the depleted saltwater withdrawn from the electrochemical cell is replenished with saltwater from the epoxidation step and re-circulated back in the electrochemical cell. In still other embodiments, the depleted saltwater withdrawn from the electrochemical cells is replenished with salt (i.e. alkali metal bromide) and saltwater from the epoxidation step and re-circulated back in the electrochemical cell.

In some embodiments, the electrolyte including the cathode electrolyte and/or the anode electrolyte and/or the third electrolyte, such as, saltwater includes water containing alkali metal bromide with more than 1% bromide content, such as, NaBr or KBr or LiBr; or more than 10% NaBr or KBr or LiBr; or more than 25% NaBr or KBr or LiBr; or more than 50% NaBr or KBr or LiBr; or more than 70% NaBr or KBr or LiBr; or between 1-99% NaBr or KBr or LiBr; or between 1-70% NaBr or KBr or LiBr; or between 1-50% NaBr or KBr or LiBr; or between 1-25% NaBr or KBr or LiBr; or between 1-10% NaBr or KBr or LiBr; or between 10-99% NaBr or KBr or LiBr; or between 10-50% NaBr or KBr or LiBr; or between 20-99% NaBr or KBr or LiBr; or between 20-50% NaBr or KBr or LiBr; or between 30-99% NaBr or KBr or LiBr; or between 30-50% NaBr or KBr or LiBr; or between 40-99% NaBr or KBr or LiBr; or between 40-50% NaBr or KBr or LiBr; or between 50-90% NaBr or KBr or LiBr; or between 60-99% NaBr or KBr or LiBr; or between 70-99% NaBr or KBr or LiBr; or between 80-99% NaBr or KBr or LiBr; or between 90-99% NaBr or KBr or LiBr; or between 90-95% NaBr or KBr or LiBr. The percentages recited herein include wt % or wt/wt % or wt/v %.

The amount of the alkali metal bromide in the anode electrolyte or in water used in the reactions herein, may be between 0.01-5M; between 0.01-4M; or between 0.01-3M; or between 0.01-2M; or between 0.01-1M; or between 0.1-4M; or between 0.1-3M; or between 0.1-2M.

In some embodiments of the methods and systems described herein, the anode electrolyte may contain an acid. The acid may be added to the anode electrolyte to bring the pH of the anolyte to 1 or 2 or less. The acid may be hydrobromic acid.

In some embodiments of the methods and systems described herein, the amount of total metal ion in the anode electrolyte or the amount of metal bromide in the anode electrolyte or the amount of copper bromide in the anode electrolyte or the amount of iron bromide in the anode electrolyte or the amount of chromium bromide in the anode electrolyte or the amount of tin bromide in the anode electrolyte or the amount of platinum bromide or the amount of metal ion that is contacted with propylene or the amount of total metal ion and the alkali metal ions (salt) in the anode electrolyte is between 0.1-12M; or between 0.1-11M; or between 0.1-10M; or between 0.1-9M; or between 0.1-8M; or between 0.1-7M; or between 0.1-6M; or between 0.1-5M; or between 0.1-4M; or between 0.1-3M; or between 0.1-2M; or between 0.1-0.5M; 1-12M; or between 1-11M; or between 1-10M; or between 1-9M; or between 1-8M; or between 1-7M; or between 1-6M; or between 1-5M; or between 1-4M; or between 1-3M; or between 1-2M; or between 2-12M; or between 2-11M; or between 2-10M; or between 2-9M; or between 2-8M; or between 2-7M; or between 2-6M; or between 2-5M; or between 2-4M; or between 2-3M; or between 3-12M; or between 3-11M; or between 3-10M; or between 3-9M; or between 3-8M; or between 3-7M; or between 3-6M; or between 3-5M; or between 3-4M; or between 4-12M; or between 4-11M; or between 4-10M; or between 4-9M; or between 4-8M; or between 4-7M; or between 4-6M; or between 4-5M; or between 5-12M; or between 5-11M; or between 5-10M; or between 5-9M; or between 5-8M; or between 5-7M; or between 5-6M; or between 6-13M; or between 6-12M; or between 6-11M; or between 6-10M; or between 6-9M; or between 6-8M; or between 6-7M; or between 7-12M; or between 7-11M; or between 7-10M; or between 7-9M; or between 7-8M; or between 8-12M; or between 8-11M; or between 8-10M; or between 8-9M; or between 9-12M; or between 9-11M; or between 9-10M; or between 10-12M; or between 10-11M; or between 11-12M. In some embodiments, the amount of total ion in the anode electrolyte, as described above, is the amount of the metal ion in the lower oxidation state plus the amount of the metal ion in the higher oxidation state plus the alkali metal bromide; or the total amount of the metal ion in the higher oxidation state; or the total amount of the metal ion in the lower oxidation state.

In some embodiments, the depleted saltwater (or the aqueous alkali metal bromide) from the cell may be circulated back to the cell. In some embodiments, the cathode electrolyte includes 1-90%; or 1-50%; or 1-40%; or 1-30%; or 1-20%; or 1-15%; or 1-10%; or 5-90%; or 5-50%; or 5-40%; or 5-30%; or 5-20%; or 5-15%; or 5-10%; or 10-90%; or 10-50%; or 10-40%; or 10-30%; or 10-20%; or 10-15%; or 15-20%; or 15-30%; or 20-30%, of the sodium hydroxide solution. In some embodiments, the anode electrolyte includes 0.5-5M; or 0.5-4.5M; or 0.5-4M; or 0.5-3.5M; or 0.5-3M; or 0.5-2.5M; or 0.5-2M; or 0.5-1.5M; or 2-5M; or 2-4.5M; or 2-4M; or 2-3.5M; or 2-3M; or 2-2.5M; or 3-5M; or 3-4.5M; or 3-4M; or 3-3.5M; or 4-5M total metal ion solution. In some embodiments, the anode does not form an oxygen gas. In some embodiments, the anode does not form a bromine gas.

Depending on the degree of alkalinity desired in the cathode electrolyte, the pH of the cathode electrolyte may be adjusted and in some embodiments is maintained between 6 and 12; or between 7 and 14 or greater; or between 7 and 13; or between 7 and 12; or between 7 and 11; or between 10 and 14 or greater; or between 10 and 13; or between 10 and 12; or between 10 and 11. In some embodiments, the pH of the cathode electrolyte may be adjusted to any value between 7 and 14 or greater, a pH less than 12, a pH 7.0, 7.5, 8.0, 8.5, 9.0, 9.5, 10.0, 10.5, 11.0, 11.5, 12.0, 12.5, 13.0, 13.5, 14.0, and/or greater.

Similarly, in some embodiments of the system, the pH of the anode electrolyte is adjusted and is maintained between 0-7; or between 0-6; or between 0-5; or between 0-4; or between 0-3; or between 0-2; or between 0-1; or less than 0. As the voltage across the anode and cathode may be dependent on several factors including the difference in pH between the anode electrolyte and the cathode electrolyte (as can be determined by the Nernst equation well known in the art), in some embodiments, the pH of the anode electrolyte may be adjusted to a value between 0 and 7, including 0, 0.5, 1.0, 1.5, 2.0, 2.5, 3.0, 3.5, 4.0, 4.5, 5.0, 5.5, 6.0, 6.5 and 7, or to a value less than 0 depending on the desired operating voltage across the anode and cathode. Thus, in equivalent systems, where it is desired to reduce the energy used and/or the voltage across the anode and cathode, e.g., as in the chlor-alkali process, the carbon dioxide or a solution containing dissolved carbon dioxide can be added to the cathode electrolyte to achieve a desired pH difference between the anode electrolyte and cathode electrolyte.

In some embodiments, the systems provided herein result in low to zero voltage systems that generate alkali as compared to chlor-alkali process or chlor-alkali process with ODC or any other process that oxidizes metal ions from lower oxidation state to the higher oxidation state in the anode chamber. In some embodiments, the electrochemical systems described herein run at voltage of less than 2.8V; or less than 2.5V; or less than 2V; or less than 1.2V; or less than 1.1V; or less than 1V; or less than 0.9V; or less than 0.8V; or less than 0.7V; or less than 0.6V; or less than 0.5V; or less than 0.4V; or less than 0.3V; or less than 0.2V; or less than 0.1V; or at zero volts; or between 0-1.2V; or between 0-1V; or between 0-0.5 V; or between 0.5-1V; or between 0.5-2V; or between 0-0.1 V; or between 0.1-1V; or between 0.1-2V; or between 0.01-0.5V; or between 0.01-1.2V; or between 1-1.2V; or between 0.2-1V; or 0V; or 0.5V; or 0.6V; or 0.7V; or 0.8V; or 0.9V; or 1V.

As used herein, the “voltage” includes a voltage or a bias applied to or drawn from an electrochemical cell that drives a desired reaction between the anode and the cathode in the electrochemical cell. In some embodiments, the desired reaction may be the electron transfer between the anode and the cathode such that an alkaline solution, water, or hydrogen gas is formed in the cathode electrolyte and the metal ion is oxidized at the anode. In some embodiments, the desired reaction may be the electron transfer between the anode and the cathode such that the metal ion in the higher oxidation state is formed in the anode electrolyte from the metal ion in the lower oxidation state. The voltage may be applied to the electrochemical cell by any means for applying the current across the anode and the cathode of the electrochemical cell. Such means are well known in the art and include, without limitation, devices, such as, electrical power source, fuel cell, device powered by sun light, device powered by wind, and combination thereof. The type of electrical power source to provide the current can be any power source known to one skilled in the art. For example, in some embodiments, the voltage may be applied by connecting the anodes and the cathodes of the cell to an external direct current (DC) power source. The power source can be an alternating current (AC) rectified into DC. The DC power source may have an adjustable voltage and current to apply a requisite amount of the voltage to the electrochemical cell.

In some embodiments, the current applied to the electrochemical cell is at least 50 mA/cm²; or at least 100 mA/cm²; or at least 150 mA/cm²; or at least 200 mA/cm²; or at least 500 mA/cm²; or at least 1000 mA/cm²; or at least 1500 mA/cm²; or at least 2000 mA/cm²; or at least 2500 mA/cm²; or between 100-2500 mA/cm²; or between 100-2000 mA/cm²; or between 100-1500 mA/cm²; or between 100-1000 mA/cm²; or between 100-500 mA/cm²; or between 200-2500 mA/cm²; or between 200-2000 mA/cm²; or between 200-1500 mA/cm²; or between 200-1000 mA/cm²; or between 200-500 mA/cm²; or between 500-2500 mA/cm²; or between 500-2000 mA/cm²; or between 500-1500 mA/cm²; or between 500-1000 mA/cm²; or between 1000-2500 mA/cm²; or between 1000-2000 mA/cm²; or between 1000-1500 mA/cm²; or between 1500-2500 mA/cm²; or between 1500-2000 mA/cm²; or between 2000-2500 mA/cm².

In some embodiments, the cell runs at voltage of between 0-3V when the applied current is 100-250 mA/cm² or 100-150 mA/cm² or 100-200 mA/cm² or 100-300 mA/cm² or 100-400 mA/cm² or 100-500 mA/cm² or 150-200 mA/cm² or 200-150 mA/cm² or 200-300 mA/cm² or 200-400 mA/cm² or 200-500 mA/cm² or 150 mA/cm² or 200 mA/cm² or 300 mA/cm² or 400 mA/cm² or 500 mA/cm² or 600 mA/cm². In some embodiments, the cell runs at between 0-1V. In some embodiments, the cell runs at between 0-1.5V when the applied current is 100-250 mA/cm² or 100-150 mA/cm² or 150-200 mA/cm² or 150 mA/cm² or 200 mA/cm². In some embodiments, the cell runs at between 0-1V at an amperic load of 100-250 mA/cm² or 100-150 mA/cm² or 150-200 mA/cm² or 150 mA/cm² or 200 mA/cm². In some embodiments, the cell runs at 0.5V at a current or an amperic load of 100-250 mA/cm² or 100-150 mA/cm² or 150-200 mA/cm² or 150 mA/cm² or 200 mA/cm².

The systems provided herein are applicable to or can be used for any of one or more methods described herein. In some embodiments, the systems provided herein further include an oxygen gas supply or delivery system operably connected to the cathode chamber. The oxygen gas delivery system is configured to provide oxygen gas to the gas-diffusion cathode. In some embodiments, the oxygen gas delivery system is configured to deliver gas to the gas-diffusion cathode where reduction of the gas is catalyzed to hydroxide ions. In some embodiments, the oxygen gas and water are reduced to hydroxide ions; un-reacted oxygen gas in the system is recovered; and re-circulated to the cathode. The oxygen gas may be supplied to the cathode using any means for directing the oxygen gas from the external source to the cathode. Such means for directing the oxygen gas from the external source to the cathode or the oxygen gas delivery system are well known in the art and include, but not limited to, pipe, duct, conduit, and the like. In some embodiments, the system or the oxygen gas delivery system includes a duct that directs the oxygen gas from the external source to the cathode. It is to be understood that the oxygen gas may be directed to the cathode from the bottom of the cell, top of the cell or sideways. In some embodiments, the oxygen gas is directed to the back side of the cathode where the oxygen gas is not in direct contact with the catholyte. In some embodiments, the oxygen gas may be directed to the cathode through multiple entry ports. The source of oxygen that provides oxygen gas to the gas-diffusion cathode, in the methods and systems provided herein, includes any source of oxygen known in the art. Such source include, without limitation, ambient air, commercial grade oxygen gas from cylinders, oxygen gas obtained by fractional distillation of liquefied air, oxygen gas obtained by passing air through a bed of zeolites, oxygen gas obtained from electrolysis of water, oxygen obtained by forcing air through ceramic membranes based on zirconium dioxides by either high pressure or electric current, chemical oxygen generators, oxygen gas as a liquid in insulated tankers, or combination thereof. In some embodiments, the oxygen from the source of oxygen gas may be purified before being administered to the cathode chamber. In some embodiments, the oxygen from the source of oxygen gas is used as is in the cathode chamber.

### Oxybromination Reaction/Reactor

In some embodiments of the above noted aspect and embodiments, the method further comprises oxybrominating the metal bromide with the metal ion in the lower oxidation state to the higher oxidation state in presence of oxidant, such as, but not limited to, oxygen (or other oxidants listed herein) optionally in the presence of HBr.

Accordingly, there are provided methods that include

(i) contacting an anode with an anode electrolyte in an electrochemical cell wherein the anode electrolyte comprises metal bromide with metal ion in a lower oxidation state, metal bromide with metal ion in a higher oxidation state, and saltwater; contacting a cathode with a cathode electrolyte in the electrochemical cell; applying voltage to the anode and the cathode and oxidizing the metal bromide with metal ion in a lower oxidation state to a higher oxidation state at the anode;

(ii) withdrawing the anode electrolyte from the electrochemical cell and brominating propylene with the anode electrolyte comprising the metal bromide with the metal ion in the higher oxidation state in the saltwater under reaction conditions to result in one or more products comprising PBH and the metal bromide with the metal ion in the lower oxidation state; or withdrawing the anode electrolyte from the electrochemical cell and brominating ethylene with the anode electrolyte comprising the metal bromide with the metal ion in the higher oxidation state in the saltwater under reaction conditions to result in one or more products comprising BE and the metal bromide with the metal ion in the lower oxidation state;

(iii) oxybrominating the metal bromide with the metal ion in the lower oxidation state in the saltwater to the higher oxidation state in presence of oxygen and, optionally HBr; and

(iv) epoxidizing the PBH or the BE with a base to form PO or EO, respectively.

The above noted aspect may further comprise hydrolysis reaction as described herein.

The “oxybromination” or its grammatical equivalent, as used herein, includes a reaction in which an oxidant oxidizes the metal ion of the metal bromide from the lower oxidation state to the higher oxidation state. In some embodiments, the oxidation of the metal ion from the lower oxidation state to the higher oxidation state may occur separately from the formation of the metal bromide with the metal in the higher oxidation state. For example, the metal bromide with the metal in the lower oxidation state may be oxidized to a metal hydroxybromide with the metal in the higher oxidation state. The metal hydroxybromide may then be converted to a metal bromide with the metal in a higher oxidation state through the addition of a source of bromine such as HBr. The metal hydroxybromide has been described in detail further herein.

The oxidant includes one or more oxidizing agents that oxidize the metal ion of the metal bromide from the lower to the higher oxidation state. Examples of oxidant include, without limitation, Oxygen or HBr gas and/or HBr solution in combination with gas comprising oxygen or ozone. Other oxidants that may be used to supplement the foregoing oxidants or used independently include, without limitation, hydrogen peroxide, HBrO or salt thereof, HBrO₃ or salt thereof, HBrO₄ or salt thereof, or combinations thereof.

The gas comprising oxygen can be any gas comprising more than 1% oxygen; or more than 5% oxygen; or more than 10% oxygen; or more than 20% oxygen; or more than 30% oxygen; or more than 40% oxygen; or more than 50% oxygen; or between 1-30% oxygen; or between 1-25% oxygen; or between 1-20% oxygen; or between 1-15% oxygen; or between 1-10% oxygen; or is atmospheric air (about 21% oxygen). In some embodiments, when oxygen depolarizing cathode (ODC) is used in the cathode chamber of the electrochemical cell (described in detail below), then the oxygen introduced in the cathode chamber may also be used for the oxybromination reaction. In some embodiments, the oxygen that exits the cathode chamber after being used at the ODC, may be collected and transferred to the oxybromination reactor for the oxybromination reaction. In some embodiments, the cathode chamber may be operably connected to the oxybromination reactor for the circulation of the oxygen gas.

In some embodiments, when the oxidant is HBr gas and/or HBr solution in combination with air, the air deprived of the oxygen (after reaction in the oxybromination reactor) and rich in nitrogen may be collected, optionally compressed, and sold in the market. In some embodiments, the air rich in nitrogen is replenished with oxygen and returned to the oxybromination reaction.

In some embodiments, the gas may comprise ozone alone or in combination with oxygen gas. In some embodiments, the gas comprising ozone can be any gas comprising more than 0.1% ozone; or more than 1% ozone; or more than 10% ozone; or more than 20% ozone; or more than 30% ozone; or more than 40% ozone; or more than 50% ozone; or between 0.1-30% ozone; or between 0.1-25% ozone; or between 0.1-20% ozone; or between 0.1-15% ozone; or between 0.1-10% ozone.

In some embodiments, the concentration of the oxidant solution (e.g. HBr) is between about 0.1-10M; or 0.1-5M; or 0.1-1M; or 5-10M; or 1-5M.

In some embodiments, the ratio of the HBr gas and/or HBr solution (I) and the gas comprising oxygen or ozone (II), i.e. I:II is 1:1 or 2:1 or 3:1 or 2:0.5 or 2:0.1 or 1:0.1 or 1:0.5.

In some embodiments, the HBr gas or HBr solution used as an oxidant is obtained from the bromination process. For example, when the propylene is brominated with CuBr₂ to form the PBH, the bromination results in the formation of HBr. The HBr thus formed may optionally be separated from the organics and may be used in the oxybromination reaction.

In some embodiments, there are provided systems that carry out the above noted method described herein.

In some embodiments, there are provided systems that include

an electrochemical cell comprising an anode in contact with an anode electrolyte wherein the anode electrolyte comprises metal bromide with metal ion in a lower oxidation state, metal bromide with metal ion in a higher oxidation state, and saltwater; a cathode in contact with a cathode electrolyte; and a voltage source configured to apply voltage to the anode and the cathode wherein the anode is configured to oxidize the metal bromide with the metal ion from a lower oxidation state to a higher oxidation state;

a bromination reactor operably connected to the electrochemical cell wherein the bromination reactor is configured to receive the metal bromide with the metal ion in the higher oxidation state from the electrochemical cell and brominate propylene or ethylene with the metal bromide with the metal ion in the higher oxidation state under reaction conditions to result in one or more products comprising PBH or one or more products comprising BE, respectively, and the metal bromide solution with the metal ion in the lower oxidation state;

an oxybromination reactor operably connected to the bromination reactor and configured to oxybrominate the metal bromide with the metal ion from the lower oxidation state to the higher oxidation state in presence of oxygen and, optionally HBr; and

an epoxide reactor operably connected to the bromination reactor and/or the oxybromination reactor and configured to epoxidize the PBH or BE with a base to form PO or EO, respectively.

In some embodiments, in the system noted above, the one or more products further comprise DBP (from propylene) or DBE (from ethylene) and the systems further comprise hydrolysis reactor operably connected to the bromination reactor and configured to hydrolyze the DBP to PBH and propanal or DBE to BE and bromoacetaldehyde. In some embodiments, the epoxide reactor is also operably connected to the hydrolysis reactor and is configured to epoxidize the PBH and propanal from the hydrolysis reactor with a base to form PO and unreacted propanal. In some embodiments, the epoxide reactor is also operably connected to the hydrolysis reactor and is configured to epoxidize the BE and bromoacetaldehyde with a base to form EO and unreacted bromoacetaldehyde.

In some embodiments, when the oxidant is HBr gas and/or HBr solution in combination with gas comprising oxygen or ozone, the HBr gas and/or HBr solution as well as the gas comprising oxygen or ozone may be administered to the oxybromination reactor. The reactor may also receive the aqueous solution of metal bromide with the metal ion in the lower oxidation state. The solution may be the anode electrolyte comprising saltwater (e.g. aqueous NaBr) and the metal bromide from the electrochemical cell or the solution may be the saltwater from the bromination reactor (which contains HBr also). The oxybromination reactor may be any column, tube, tank, pipe, or reactors that can carry out the oxybromination reaction. The reactor may be fitted with various probes including temperature probe, pH probe, pressure probe, etc. to monitor the reaction. The reaction may be heated with means to heat the reaction mixture. The temperature of the reactor may be between about 40-200° C.; or between about 40-160° C.; or between about 60-150° C.; or between about 60-100° C.; or between about 50-150° C.; or between about 50-100° C.; or between about 60-90° C.; or between about 65-90° C.; or between about 60-85° C.; or between about 65-90° C.; or between about 50-90° C. The pressure in the oxybromination reactor may be between about 1-300 psig; or between about 1-200 psig; or between about 1-100 psig; or between about 1-75 psig; or between about 1-50 psig; or between about 1-30 psig; or between about 1-10 psig; or between about 10-100 psig; or between about 10-50 psig. In some embodiments, oxygen partial pressure in the feed to the oxybromination method and system is in a range between about 0.01-300 psia; or between about 0.01-200 psia; or between about 0.01-100 psia; or between about 0.01-50 psia; or between about 0.01-30 psia; or between about 0.1-300 psia; or between about 0.1-200 psia; or between about 0.1-100 psia; or between about 0.1-50 psia; or between about 0.1-30 psia; or between about 1-300 psia; or between about 1-200 psia; or between about 1-100 psia; or between about 1-50 psia; or between about 1-30 psia. The oxybromination reaction may be carried out for between about 1 min to a few hours. The oxybromination reactor may also be fitted with conduits for the entry and/or exit of the solutions and the gases. Other detailed descriptions of the reactor are provided herein.

In some embodiments of the above noted aspect and embodiments, reaction conditions for the oxybromination reaction comprise temperature between about 50-100° C.; pressure between about 1-100 psig; oxygen partial pressure in feed to the oxybromination in a range between about 0.01-100 psia; or combinations thereof.

In some embodiments of the above noted system, the system further comprises means for transferring the HBr and the metal bromide in the lower oxidation state, formed in the bromination reactor to the oxybromination reactor and/or means for transferring the metal bromide in the higher oxidation state from the oxybromination reactor to the bromination reactor. Such means include any means for transferring liquids including, but not limited to, conduits, tanks, pipes, and the like.

These aspects and embodiments are illustrated in FIGS. 4A and 4B, where the CuBr and HBr generated in the bromination reaction/reactor are subjected to oxybromination reaction/reactor in the presence of oxygen (or any other oxidizing gas) to oxidize CuBr back to CuBr₂. The CuBr₂ can then be recirculated back to the bromination reaction for the bromination of the propylene or the ethylene. As illustrated in FIGS. 4A and 4B, CuBr is oxidized to CuBr₂ in the anode chamber of the electrochemical cell. The saltwater from the anode chamber of the electrochemical cell containing the CuBr₂ is transferred to the bromination reaction/reactor where a reaction with the propylene (C₃H₆) or reaction with the ethylene (C₂H₄) produces one or more products comprising PBH or BE, respectively, and the CuBr₂ is reduced to the CuBr. The aqueous solution from the bromination reaction/reactor containing the CuBr (also containing CuBr₂) is separated from the PBH or the BE and is transferred to the oxybromination reaction/reactor where the HBr and oxygen (or any other oxidizing gas such as ozone) oxidizes the CuBr to CuBr₂. In some embodimnets, the aqueous solution from the bromination reaction/reactor containing the CuBr (also containing CuBr₂) is not separated from the PBH or the BE and the whole solution is transferred to the oxybromination reaction/reactor where the oxygen (or any other oxidizing gas such as ozone) oxidizes the CuBr to CuBr₂. The CuBr₂ solution (also containing CuBr) is then transferred from the oxybromination reaction/reactor back to the bromination reaction/reactor. The one or more products comprising PBH or the BE (may optionally include other organic products) are then transferred from the bromination reaction/reactor and/or the oxybromination reaction/reactor (after separation) to the epoxidation reaction/reactor.

The methods illustrated in FIGS. 4A and 4B use the HBr generated in the bromination reaction as a source of a bromide ion for an oxybromination step. The oxybromination step now regenerates half of the CuBr₂ for the bromination reaction, while the electrochemical cell regenerates the other half of CuBr₂. As a result, the electrochemical cell's power demand is cut in half when compared to the method illustrated in FIGS. 3A and 3B. For example only, compared to a chlor-alkali unit operating at about 3V (to generate Cl₂ for chlorination), the electrochemical cell in FIGS. 4A and 4B may effectively be operating at about 2.6V or between about 2.4-2.8V; or between about 2.4-2.5V; or between about 2.4-2.6V; or between about 2.4-2.7V; or between about 2.4-2.8V; or between about 2.5-2.6V; or between about 2.5-2.7V; or between about 2.5-2.8V; or between about 2.6-2.7V; or between about 2.6-2.8V; or between about 2.7-2.8V; or between about 2-3V, but half as many cells would be needed. In addition, there may be savings in salt demand and cell CapEx.

FIGS. 4A and 4B illustrate oxybromination using HBr and oxygen. Any other oxidant as listed herein, may be used for the oxybromination reaction. In some embodiments, the oxidant is HBr gas and/or HBr solution in combination with hydrogen peroxide. One example is as follows:

2CuBr+H₂O₂+2HBr→2CuBr₂+2H₂O

The oxidants have been described in U.S. patent application Ser. No. 15/963,637, filed Apr. 26, 2018, which is incorporated herein by reference in its entirety. The methods and systems provided herein can leverage the HBr in the oxybromination step as a mechanism to provide additional copper oxidation. The HBr can also be sourced from other reactions and may be referred to as “other HBr”. The incorporation of HBr from the bromination reaction or other reactions may lead to additional PO production by upgrading these streams to more valuable products. The reuse of the HBr in the oxybromination process allows for the reduction of the base consumption (e.g. NaOH) to neutralize the acid which may improve overall economics, especially in cases where the base could otherwise be sold.

In some embodiments of the above noted aspects and embodiments, the concentration of the metal bromide with the metal ion in the lower oxidation state entering the oxybromination reaction is between about 0.3-2M; or between about 0.3-1.5M; or between about 0.3-1M; or between about 0.3-0.5M; or between about 0.5-2M; or between about 0.5-1.5M; or between about 0.5-1M; or between about 0.5-0.5M; or between about 1-2M; or between about 1-1.5M.

In some embodiments, the above noted system further comprises a conduit or a pipe or a delivery system (fitted with valves etc.) operably connected between the bromination reactor and the oxybromination reactor configured for delivering the metal bromide with the metal ion in the lower oxidation state in the saltwater of the bromination reactor to the oxybromination reactor wherein the oxybromination reactor oxybrominates the metal bromide with the metal ion from the lower oxidation state to the higher oxidation state. In some embodiments, the system further comprises a conduit or a pipe or a delivery system (fitted with valves etc.) operably connected between the oxybromination reactor and the bromination reactor configured for delivering the metal bromide with the metal ion in the higher oxidation state in the saltwater of the oxybromination reactor to the bromination reactor. In some embodiments, the system further comprises a separator (not shown in the figures) operably connected to the bromination reactor and/or the oxybromination reactor configured to receive the solution of the one or more products and the metal bromide with the metal ion in the lower oxidation state from the bromination reactor, and to separate the one or more products from the metal bromide in the saltwater after the bromination reactor. In some embodiments, the separator is further configured to deliver the metal bromide with the metal ion in the lower oxidation state to the oxybromination reactor and/or the electrochemical cells and the one or more products comprising PBH or BE to the epoxidation reactor. The aqueous solution (or the saltwater) containing the metal bromide with the metal ion in the lower oxidation state separated from the one or more products further includes HBr for oxybromination. Various separation and purification methods and systems have been described in U.S. patent application Ser. No. 14/446,791, filed Jul. 30, 2014, which is incorporated herein by reference in its entirety in the present disclosure. Some examples of the separation techniques include without limitation, reactive distillation, adsorbents, liquid-liquid separation, liquid-vapor separation, etc.

It is to be understood that the processes, such as the electrochemical reaction, the bromination reaction, the hydrolysis reaction, and the oxybromination reaction, may each be individually carried out or may be in combination with one or more other processes. For example, the electrochemically generated CuBr₂ may be used in one reactor for the bromination of the propylene to the PBH and/or the DBP and the chemically generated CuBr₂ (via oxybromination) may be used in another propylene bromination reactor each with the option of making the PBH directly or making the DBP with subsequent conversion to the PBH in the hydrolysis reaction, all such configurations are within the scope of the present disclosure. The flow of copper bromide between the electrochemical, the bromination, the hydrolysis, and the oxybromination systems may be either clockwise or counter clockwise or in any other order. That is, the order of operations between the three units is flexible.

The examples of conduits include, without limitation, pipes, tubes, tanks, and other means for transferring the liquid solutions. In some embodiments, the conduits attached to the systems also include means for transferring gases such as, but not limited to, pipes, tubes, tanks, and the like. The gases include, for example only, the propylene gas or the ethylene gas to the bromination reactor, the oxygen or the ozone gas to the oxybromination reactor, or the oxygen gas to the cathode chamber of the electrochemical cell etc.

In one aspect, there is provided a method that includes

(i) oxybrominating a metal bromide with the metal ion in a lower oxidation state to a higher oxidation state in presence of oxygen and, optionally HBr;

(ii) withdrawing the metal bromide with the metal ion in the higher oxidation state and brominating propylene with the metal bromide with the metal ion in the higher oxidation state to result in one or more products comprising PBH and the metal bromide with the metal ion in the lower oxidation state; or withdrawing the metal bromide with the metal ion in the higher oxidation state and brominating ethylene with the metal bromide with the metal ion in the higher oxidation state to result in one or more products comprising BE and the metal bromide with the metal ion in the lower oxidation state; and

(iii) epoxidizing the PBH or the BE with a base to form PO or EO, respectively.

In some embodiments, the aforementioned one or more products further comprises DBP from propylene or DBE from ethylene and the method further comprises hydrolyzing the DBP under one or more reaction conditions to form hydrolysis products comprising PBH and propanal or hydrolyzing the DBE under one or more reaction conditions to form hydrolysis products comprising BE and bromoacetaldehyde. In some embodiments, the hydrolysis products comprising PBH and propanal or the hydrolysis products comprising BE and bromoacetaldehyde are transferred to the epoxidation reaction where the PBH and the propanal or the BE and the bromoacetaldehyde with a base form PO and unreacted propanal or EO and unreacted bromoacetaldehyde, respectively.

In some embodiments, there are provided systems that carry out the above noted method described herein.

In some embodiments, there are provided systems that include

an oxybromination reactor operably connected to a bromination reactor and configured to oxybrominate metal bromide with metal ion from lower oxidation state to higher oxidation state in presence of oxygen and, optionally HBr;

a bromination reactor operably connected to the oxybromination reactor wherein the bromination reactor is configured to receive the metal bromide with the metal ion in the higher oxidation state from the oxybromination reactor and brominates propylene or ethylene with the metal bromide with the metal ion in the higher oxidation state to result in one or more products comprising PBH or one or more products comprising BE, respectively, and the metal bromide solution with the metal ion in the lower oxidation state; and

an epoxide reactor operably connected to the bromination reactor and configured to epoxidize PBH or BE with a base to form PO or EO, respectively.

In some embodiments, the aforementioned one or more products further comprises DBP from propylene or BE from ethylene and the system further comprises hydrolysis reactor operably connected to the bromination reactor and/or the epoxide reactor and configured to hydrolyze the DBP under one or more reaction conditions to form hydrolysis products comprising PBH and propanal or hydrolyze the DBE under one or more reaction conditions to form hydrolysis products comprising BE and bromoacetaldehyde.

The above noted aspect and embodiments are illustrated in FIGS. 5A and 5B. The above noted aspect eliminates electrochemical reaction. The methods illustrated in FIGS. 5A and 5B, include formation of the DBP or the DBE in the bromination reaction and its subsequent hydrolysis to the PBH and propanal or the BE and bromoacetaldehyde respectively, in the hydrolysis step (described further herein below). It is to be understood that no DBP or no DBE may be formed, and the PBH or BE may be formed directly in the bromination reactor; or the DBP or DBE may convert to the PBH or BE respectively in situ in the presence of water; or the DBP or DBE may be separated and hydrolyzed to the PBH or BE respectively as illustrated in FIGS. 5A and 5B. All of these embodiments are well within the scope of the invention. In some embodiments, the HBr produced after hydrolysis is recirculated back to the oxybromination reaction.

In the method above, caustic may be purchased but would still be only half of the original PO or EO plants shown in FIGS. 3A and 3B. The above noted process eliminates the bromine purchase from a bromine production system (effectively debottlenecking any processes constrained by bromine capacity) and cuts the caustic consumption in half. The same amount of propylene or ethylene may still be consumed with a purchase of only one mole of HBr and half a mole of oxygen (O₂). The CapEx for this retrofit may be minimized because there are no cells to purchase.

Oxidation with Bromine Gas

In some embodiments of the above noted aspect, the electrochemical oxidation of the metal bromide with the metal ion in the lower oxidation state to the higher oxidation state, e.g. CuBr to CuBr₂, as illustrated in FIGS. 3A, 3B, 4A, 4B, 7 and 8, may be replaced by oxidation of the metal bromide with the metal ion in the lower oxidation state to the higher oxidation state, e.g. CuBr to CuBr₂ with elemental bromine (Br₂). For example, in some embodiments, traditional bromine generating processes, such as those which produce bromine from bromine containing brines, may be retro-fitted with the bromination, the oxybromination, and the epoxidation reactors of the invention in order to produce the PO from the propylene or the EO from the ethylene. In some situations, the operators may save on the investment cost by using the existing bromine production facility. Because the outlet of the epoxidation reaction produces a stream that is rich in NaBr, this stream may then be recycled to the inlet brine stream of a bromine generating facilities where the bromide ions would again be converted to molecular bromine. In this way, the process may not result in a net consumption of bromine. Those skilled in the art will readily recognize that existing chlor-alkali facilities would also have an opportunity to retrofit chlorine-producing facilities to generate EO and PO based on a similar process. In this case, the elemental chlorine would be used to generate molecular bromine (Br₂) from the sodium bromide (NaBr) generated in the epoxidation reaction through the displacement reaction 2NaBr+Cl₂→2NaCl+Br₂. The molecular bromine would then be used to convert CuBr to CuBr₂ and the process implemented as discussed above.

In one aspect, there is provided a method that includes

(i) contacting molecular bromine with a solution comprising metal bromide and oxidizing the metal bromide with metal ion in a lower oxidation state to a higher oxidation state with the bromine gas;

(ii) brominating propylene with the metal bromide with the metal ion in the higher oxidation state in the solution to result in one or more products comprising PBH and the metal bromide with the metal ion in the lower oxidation state; or brominating ethylene with the metal bromide with the metal ion in the higher oxidation state in the solution to result in one or more products comprising BE and the metal bromide with the metal ion in the lower oxidation state; and

(iii) epoxidizing the PBH or the BE with a base to form PO or EO, respectively.

In some embodiments, the aforementioned one or more products further comprises DBP from propylene or DBE from ethylene and the method further comprises hydrolyzing the DBP under one or more reaction conditions to form hydrolysis products comprising PBH and propanal or hydrolyzing the DBE under one or more reaction conditions to form hydrolysis products comprising BE and bromoacetaldehyde. The hydrolysis products may be then transferred to the epoxide reaction to form PO and unreacted propanal or EO and unreacted bromoacetaldehyde, respectively.

In some embodiments of the above noted aspect, the method further includes treating the brine (e.g. aq. NaBr) formed in the epoxidation reaction with chlorine to form molecular bromine and transferring the molecular bromine to step (i). In some embodiments of the above noted aspect and embodiments, the method further includes oxybrominating the metal bromide from the lower oxidation state to the higher oxidation state in the presence of the oxidant (as illustrated in FIGS. 6A and 6B).

In some embodiments, there are provided systems that carry out the above noted methods.

In some embodiments, there are provided systems that include

an oxidation reactor configured to oxidize metal bromide with metal ion from lower oxidation state to higher oxidation state in presence of molecular bromine;

a bromination reactor operably connected to the oxidation reactor wherein the bromination reactor is configured to receive the metal bromide with the metal ion in the higher oxidation state from the oxidation reactor and brominate propylene or ethylene with the metal bromide with the metal ion in the higher oxidation state to result in one or more products comprising PBH or one or more products comprising BE, respectively, and the metal bromide solution with the metal ion in the lower oxidation state; and

an epoxide reactor operably connected to the bromination reactor and configured to epoxidize PBH or BE with a base to form PO or EO, respectively.

In some embodiments, the aforementioned one or more products further comprises DBP from propylene or BE from ethylene and the system further comprises hydrolysis reactor operably connected to the bromination reactor and configured to hydrolyze the DBP under one or more reaction conditions to form hydrolysis products comprising PBH and propanal or hydrolysis reactor operably connected to the bromination reactor and configured to hydrolyze the DBE under one or more reaction conditions to form hydrolysis products comprising BE and bromoacetaldehyde. The hydrolysis products may be then transferred to the epoxide reactor to form PO and unreacted propanal or EO and unreacted bromoacetaldehyde, respectively.

In some embodiments of the above noted aspect, the system further comprises an oxybromination reactor operably connected to a bromination reactor and configured to oxybrominate metal bromide with metal ion from lower oxidation state to higher oxidation state in presence of HBr and oxygen. In some embodiments of the above noted aspect and embodiments, the system further includes a reactor operably connected to the epoxidation reactor and configured for treating the brine (e.g. aq. NaBr) formed in the epoxidation reactor with chlorine to form molecular bromine and further configured for transferring the molecular bromine to the oxidation reactor.

In some embodiments of the systems described herein, the system further comprises a hydrolyzing chamber operably connected to the bromination reactor and configured to receive the DBP or DBE from the bromination reactor and/or the epoxide reactor and hydrolyze the DBP to PBH and propanal or hydrolyze the DBE to BE and bromoacetaldehyde. In some embodiments, the hydrolyzing chamber is also operably connected to the epoxide reactor and is configured to transfer PBH or the BE to the epoxide reactor. The oxybromination reaction/reactor; the hydrolyzing reaction/chamber and epoxide reaction/reactor, have been all described in detail herein.

In some embodiments of the above noted system, the system further comprises means for transferring solutions in between the reactors. Such means include any means for transferring liquids including, but not limited to, conduits, tanks, pipes, and the like.

The above noted aspect is illustrated in FIGS. 6A and 6B. As explained, the above noted aspect eliminates electrochemical reaction of the invention but replaces it with the electrolyzer that produces bromine. The methods illustrated in FIGS. 6A and 6B, illustrate the electrochemical reaction of the electrolyzer that produces NaOH, H₂, and Br₂. In the oxidation reactor, the CuBr is converted to CuBr₂ by the direct addition of Br₂. This reaction may take place in a slurry reactor or in a liquid phase reactor where bromine is injected directly into the liquid or slurry. The outlet of this reactor may feed the bromination reactor where PBH or BE is generated from propylene or ethylene and CuBr₂. The PBH or BE may be then separated from the aqueous stream and sent to the epoxidation reactor. The residual aqueous copper bromide stream (liquid or slurry) then may feed the oxybromination reactor where CuBr may be converted to CuBr₂ via the reaction shown in FIGS. 6A and 6B. The oxybromination and the epoxidation reactions have been described in detail herein. The process to form bromine is shown as an illustrative example only; any source of bromine can be used to carry out the methods and systems provided herein. Furthermore, while CaO is illustrated as a base for the epoxidation reaction in FIGS. 6A and 6B, it is to be understood that the NaOH formed in the electrochemical reaction can also be used as the base for the epoxidation reaction and the aqueous brine stream exiting the epoxidation reaction may be fed back to the electrochemical cell. The embodiments that include this use of NaOH from the electrochemical cell/reaction to the epoxidation reaction/reactor have been illustrated in FIGS. 3A, 3B, 4A, and 4B.

Depending on the downstream usage, bromine produced in the electrolyzer may be dried or may be used directly without drying. In some embodiments, waste HBr from other processes may be provided to the oxybromination unit. Such chemical processes include, but not limited to, ethylene dibromide (EDB) cracking and phosgene based reactions where HBr may be generated as a by-product.

Although not shown in FIGS. 6A and 6B, the copper bromide stream may be fed from the oxybromination reactor to the oxidation reactor or vice versa.

In some of the above noted aspects and embodiments, the oxidizing, the brominating, the oxybrominating, and the epoxidizing steps are all carried out in saltwater.

In some embodiments of the method and system aspects and embodiments provided herein, the concentration of the metal bromide with the metal ion in the lower oxidation state, the concentration of the metal bromide with the metal ion in the higher oxidation state, and the concentration of the salt in the water (e.g. alkali metal bromide), each individually or collectively may affect the performance of each of the electrochemical cell/reaction, oxybromination reactor/reaction, and bromination reactor/reaction and also affect the STY (space time yield) and selectivity of PBH or BE. Since the electrochemical cell/reaction, oxybromination reactor/reaction, and bromination reactor/reaction are interconnected in various combinations in the present invention, it was found that the concentrations of the metal bromide with lower and higher oxidation state and the salt concentration exiting the systems/reactions and entering the systems/reactions may affect the performance, yield, selectivity, STY, and/or voltage as applicable to the systems.

In some of the above noted aspects and embodiments (as appropriate to the combination), concentration of the metal bromide with the metal ion in the lower oxidation state entering the oxybromination reaction is between about 0.3-2M; concentration of the metal bromide with the metal ion in the lower oxidation state entering the bromination reaction is between about 0.01-2M; concentration of the metal bromide with the metal ion in the lower oxidation state entering the electrochemical reaction is between about 0.3-2.5M; or combinations thereof.

In some of the above noted aspects and embodiments, the methods further comprise separating the metal bromide solution from the one or more products comprising PBH or BE after the brominating step and delivering the metal bromide solution back to the electrochemical reaction and/or the oxybromination reaction.

In some of the above noted aspects and embodiments, the yield of the PO is more than 90 wt % or more than 92 wt % or more than 95 wt % and/or the space time yield (STY) of the PO is more than 0.1, or more than 0.5, or 1 (mol/L/hr). In some of the above noted aspects and embodiments, the yield of the EO is more than 90 wt % or more than 92 wt % or more than 95 wt % and/or the space time yield (STY) of the EO is more than 0.1, or more than 0.5, or 1 (mol/L/hr).

In some embodiments of the aforementioned aspect, when the electrochemical cell, the bromination reactor and/or the oxybromination reactor are operably connected (depending on the combinations described herein) to the other systems, the systems further comprises a conduit or a pipe or a delivery system (fitted with valves etc.) operably connected between the reactors or systems configured to deliver the one or more products, the saltwater and the metal bromides from one reactor or system to the other. For example, in some embodiments, the system further comprises a conduit or a pipe or a delivery system (fitted with valves etc.) operably connected between the oxybromination reactor and the bromination reactor (e.g. in FIGS. 5A and 5B) and configured to deliver the metal bromide solution containing the metal ion in the higher oxidation state and the saltwater of the oxybromination reactor to the bromination reactor for the bromination of the propylene or ethylene to form the one or more products.

In some embodiments, the system further comprises a separator operably connected to the bromination reactor and configured to separate the one or more products from the metal bromide in the saltwater after the bromination reactor. In some embodiments, the separator is further configured to deliver the metal bromide solution with the metal ion in the lower oxidation state and the higher oxidation state to the electrochemical cell and/or the oxybromination reactor. In some embodiments, the system further comprises a conduit or a pipe or a delivery system (fitted with valves etc.) operably connected between the bromination reactor and the electrochemical cell/the oxybromination reactor and configured to recirculate back the saltwater after the bromination. Further, in some embodiments, the system further comprises a conduit or a pipe or a delivery system (fitted with valves etc.) operably connected between the oxybromination reactor or the bromination reactor and the epoxidation reactor and configured to deliver the PBH and propanal or the BE and bromoacetaldehyde after separation, to the epoxidation reactor for the formation of PO and unreacted propanal or EO and unreacted bromoacetaldehyde respectively. The examples of conduits include, without limitation, pipes, tubes, tanks, and other means for transferring the liquid solutions. In some embodiments, the conduits attached to the systems also include means for transferring gases such as, but not limited to, pipes, tubes, tanks, and the like. The gases include, for example only, the propylene or the ethylene to the bromination reactor, the oxygen or the ozone gas to the oxybromination reactor, or the oxygen gas to the cathode chamber of the electrochemical cell etc.

In all the systems provided herein, the solution in and out of the systems may be recirculated multiple times before sending the solution to the next system. For example, when the oxybromination reactor is operably connected to the bromination reactor, the saltwater from the oxybromination reactor may be sent back to the bromination reactor or is circulated between the oxybromination and the bromination reactor before the solution is taken out of the oxybromination system and sent to the bromination reactor or any other reactor.

In all the systems provided herein, the use of oxybromination may be varied with time throughout the day. For example, the oxybromination may be run during peak power price times as compared to electrochemical reaction thereby reducing the energy use. For example, oxybromination may be run in the day time while the electrochemical cell may be run in the night time in order to save the cost of energy.

In some embodiments, the saltwater containing the one or more products and the metal bromide may be subjected to washing step which may include rinsing with an organic solvent or passing the organic product through a column to remove the metal ions. In some embodiments, the organic products may be purified by distillation. In the methods and systems provided herein, the separation and/or purification may include one or more of the separation and purification of the organic products from the metal ion solution; the separation and purification of the organic products from each other; and separation and purification of the metal ion in the lower oxidation state from the metal ion in the higher oxidation state, to improve the overall yield of the organic product, improve selectivity of the organic product, improve purity of the organic product, improve efficiency of the systems, improve ease of use of the solutions in the overall process, improve reuse of the metal solution in the electrochemical and reaction process, and to improve the overall economics of the process. Various methods of separation/purification have been described in US Patent Application Publication No. 2015/0038750, filed Jul. 30, 2014, which is incorporated herein by reference in its entirety.

In some embodiments of the foregoing aspects and embodiments, the yield of PBH and PO or of the BE and EO obtained by using one or more aforementioned combinations of the electrochemical method/system, bromination method/system, oxybromination method/system, and/or epoxidation method/system is more than 10 wt % yield; or more than 20 wt % yield; or more than 30 wt % yield; or more than 40 wt % yield; or more than 50 wt % yield; or more than 60 wt % yield; or more than 70 wt % yield; or more than 80 wt % yield; or more than 90 wt % yield; or more than 95 wt % yield; or between 20-90 wt % yield; or between 40-90 wt % yield; or between 50-90 wt % yield, or between 50-99 wt % yield.

In some embodiments of the foregoing aspects and embodiments, the STY (space time yield) of PBH and PO or of the BE and EO, obtained by using one or more aforementioned combinations of the electrochemical method/system, bromination method/system, oxybromination method/system, and/or epoxidation method/system, is more than 0.1, or more than 0.5, or is 1, or more than 1, or more than 2, or more than 3, or more than 4, or more than 5, or between 0.1-3, or between 0.5-3, or between 0.5-2, or between 0.5-1, or between 3-5, or between 3-6, or between 3-8. As used herein the STY is yield per time unit per reactor volume (mol/L/hr). For example, the yield of product may be expressed in mol, the time unit in hour and the volume in liter. The volume may be the nominal volume of the reactor, e.g. in a packed bed reactor, the volume of the vessel that holds the packed bed is the volume of the reactor. The STY may also be expressed as STY based on the consumption of propylene or ethylene to form the product. For example only, in some embodiments, the STY of the product may be deduced from the amount of propylene or ethylene consumed during the reaction. The selectivity may be the mol of product/mol of the propylene or ethylene consumed (e.g. only, mol PBH made/mol propylene consumed or mol BE made/mol ethylene consumed). The yield may be the amount of the product isolated. The purity may be the amount of the product/total amount of all products (e.g. only, amount of PBH or BE/all the organic products formed).

Forming PO from PBH or EO from BE

In some embodiments of the foregoing aspect and embodiments, the methods further comprise reacting the PBH and propanal (and optionally DBP) or BE and bromoacetaldehyde (and optionally DBE) with a base to form the PO or EO, respectively. Various process configurations that lead to the epoxidation step (illustrated in FIGS. 1A, 1B, 3A, 3B, 4A, 4B, 5A, 5B, 6A, and 6B) have been described herein.

Industrial plants do not typically use a bromohydrin molecule to form PO because of prohibitively expensive economic as well as environmental cost of sodium bromide. Rather industrial formation of PO is via propylene chlorohydrin. In such cases, the conversion to the PO is a ring-closing reaction whereby the chlorohydrin molecule may be combined in a near stoichiometric ratio with a base such as e.g. sodium hydroxide (NaOH) or lime (CaO). The products are PO, the chloride salt of the base (e.g. NaCl or CaCl₂ respectively) and water. Because the PO may be a reactive molecule, it may need to be removed from the reaction media quickly. Typically, the short residence time requirement may be achieved by steam stripping the PO as it is formed in the reactor. However, because the PCH feeding the reactor may be diluted with a large excess of water due to upstream reaction selectivity considerations (described further herein below), the steam demand for PO stripping may be very high.

Applicants have devised a zero discharge system where the sodium bromide remaining in the epoxidation reactor/reaction is re-circulated back to the electrochemical cell/reaction (as shown in the figures). This zero discharge system circumvents the aforementioned disadvantage of the prohibitive cost of the sodium bromide discharge in the PO process. As described earlier herein, the bromide methods and systems described herein provide an added advantage of the ease of separation of the PO from the side products as the closest brominated C3 has a boiling point that is 13° C. away from the PO and that compound, e.g. 2-bromopropene is not made in detectable quantities.

In some aspects noted above, there are provided methods and systems comprising reacting the PBH with a base to form PO in presence of DBP and/or propanal or the methods and systems comprise reacting the solution of the PBH, the propanal, and the DBP with a base to form PO and unreacted propanal and/or unreacted DBP. Also provided are methods and systems comprising reacting the BE with a base to form EO in presence of DBE and bromoacetaldehyde (optionally other brominated derivatives may also be present) or the methods and systems comprise reacting the solution of the BE, bromoacetaldehyde and the DBE with a base to form EO and unreacted DBE and/or unreacted bromoacetaldehyde. In these aspects, the DBP (or DBE) is not separated from the PBH or propanal (or BE and bromoacetaldehyde) and the solution is directly subjected to epoxidation. In such embodiments, the separation of the DBP, the propanal, and the PBH step (or the separation of the DBE, the bromoacetaldehyde, and the BE step) may be combined with the epoxidation step such that when the base is added into the epoxidation reactor, the base reacts with the PBH to form the PO (or the base reacts with BE to form EO), which may leave the reactor as a vapor. In this process, some DBP may be converted to the PBH (or some DBE may be converted to the BE to further form EO) which would also form the PO. In some embodiments, the residual levels of unreacted PBH may leave the reactor in the DBP extraction solvent (DBP as an extraction solvent has been described before) and return to the process where appropriate. In some embodiments, the unreacted propanal or the unreacted bromoacetaldehyde may be isolated and commercially sold.

The methods and systems provided herein for converting the PBH to the PO in the presence of the DBP and propanal (where the mol % of the DBP may be equal to or greater than the mol % of the PBH) has a number of advantages. The advantages laid out here also apply to the conversion of the BE to the EO in the presence of the DBE and bromoacetaldehyde. First, it may obviate the need for separation of the PBH and the propanal from the DBP prior to the epoxidation. To maintain high selectivity of the PBH during the hydrolysis reaction, the DBP level may be in excess relative to the converted amount of the DBP as described above. The PBH may be separated from the DBP via a typical separation operation. If PBH were the lighter (lower boiling) component, distillation would be an option. However, because PBH is the heavier component, separation by distillation may require the excess DBP be removed in the overhead of the column which in turn may lead to prohibitive steam demand. Alternative separation technologies, such as absorption or selective permeation, may be equally prohibitive due to either capital equipment costs or operating costs. Second, because the PO may also be soluble in the DBP, the reactor may not require steam stripping inside the reactor. The PO can be removed from the reactor in the DBP phase if desired and separated downstream. Third, additional side reactions may be minimized because PO may react much more slowly in the organic (DBP) phase. Finally, the total waste water demand may be significantly reduced because the water leaving the reactor would primarily be that which came in with the caustic (and low levels of soluble water with the organic phase). In some embodiments, when using NaOH as the base for the PO formation, the resulting aqueous solution may be concentrated enough in NaBr to merit removing the waste organics and using the brine back in the electrochemical cell. Similarly, in other embodiments, when using other hydroxides such as KOH or LiOH as the base for the PO formation, the resulting aqueous solution may be concentrated enough in KBr or LiBr, respectively, to merit removing the waste organics and using the brine back in the electrochemical cell.

In addition to the advantages described above, the conversion of the PBH to the PO in the presence of DBP and propanal may also allow for process options that minimize by-product losses, such as, a single aqueous phase reactor that contains both reactants and products; minimizing by-product formation by running the reactor with a short residence time; step-wise addition of the NaOH; and recycling of the product stream back to the reactor. The step-wise addition of NaOH (e.g. along a length of pipe if the reaction is done in a continuous system) may reduce the by-product formation because the aqueous salt solutions resulting from the early additions may dilute the later additions. In this way, the caustic concentrations within the aqueous phase can be more easily managed along the reactor length. The recycling of the aqueous product stream back to the reactor inlet may also minimize the NaOH concentration in the aqueous phase. The recycling option has other advantages too. For example, the recycle stream may return salt-rich brine to the reactor. The presence of the salt may minimize the solubility of the PO in the aqueous phase which may improve reactor selectivity. Further, the highly concentrated salt may be advantageous because the resulting brine stream exiting the epoxidation unit may serve as a feedstock for electrolysis cells after removal of the residual, soluble organics. Furthermore, the recycle of reactor outlet may allow the reactor to run in such a way as to produce a high salt concentration outlet stream without having to feed a high concentration NaOH stream directly to the reactor. The other advantages of the high salt concentration outlet stream have also been described further herein.

In some embodiments of the foregoing aspect and embodiments, the base is an alkali metal hydroxide, such as e.g. NaOH, KOH, etc. or alkali metal oxide; alkali earth metal hydroxide or oxide, such as e.g. Ca(OH)₂ or CaO; or metal hydroxide bromide (for example only, Mₓⁿ⁺Br_{y}(OH)_{(nx-y)}). In some embodiments of the foregoing aspect and embodiments, metal in the metal hydroxybromide is same as metal in the metal bromide. In some embodiments of the foregoing aspect and embodiments, the method further comprises forming the metal hydroxybromide by oxybrominating the metal bromide with the metal ion in the lower oxidation state to the higher oxidation state in presence of water and oxygen (as explained herein).

Typically, in chlorohydrin processes for the production of propylene oxide, the NaOH may be combined and reacted with an approximately 4-5 wt % solution of propylene chlorohydrins. The propylene chlorohydrins are a mix of 1-chloro-2-propanol (approximately 85-90%) and 2-chloro-1-propanol (approximately 10-15%). The propylene oxide formation reaction is shown as below:

C₃H₆(OH)Cl+NaOH→C₃H₆O(PO)+NaCl+H₂O

Propylene oxide may be rapidly stripped from the solution in either a vacuum stripper or steam stripper. A primary disadvantage of the process may be the generation of a dilute NaCl brine stream with about 3-6 wt % NaCl with flow rate exceeding 40-45 tonnes of brine per tonne of propylene oxide. The large amount of dilute brine may result in large amount of waste water. The reason for the large volume of water may be that the reactor producing the propylene chlorohydrins must operate at dilute concentrations of about 4-5 wt % propylene chlorohydrin in order to achieve high selectivity.

Applicants have found that using the methods of the invention that produce PBH and propanal or BE and bromoacetaldehyde in high selectivity and high STY, the amount of dilute brine generated after the PO or EO formation can be eliminated or substantially reduced. In some embodiments of the foregoing aspect and embodiments, the reaction forms between about 0-40 tonnes of brine per tonne of PO or EO; or between about 0-30 tonnes of brine per tonne of PO or EO; or between about 0-20 tonnes of brine per tonne of PO or EO; or between about 0-10 tonnes of brine per tonne of PO or EO; or 3-40 tonnes of brine per tonne of PO or EO; or 3-30 tonnes of brine per tonne of PO or EO; or 3-20 tonnes of brine per tonne of PO or EO; or 3-10 tonnes of brine per tonne of PO or EO; or 0-5 tonnes of brine per tonne of PO or EO which is nil or substantially less brine compared to the brine generated in a typical PO or EO reaction. This brine may either be disposed of as waste water, recycled to the electrochemical cell, or utilized in another process such as the process to generate molecular bromine by displacement with chlorine.

In one aspect, there is provided a method to form PO, comprising brominating propylene in an aqueous medium comprising metal bromide with metal ion in higher oxidation state and salt to result in one or more products comprising between about 5-99.9 wt % PBH, and the metal bromide with the metal ion in lower oxidation state; and reacting the PBH with a base to form PO and brine in water, wherein the reaction forms between about 0-42 tonnes of brine per tonne of PO or any other range of brine per tonne of PO as provided herein. In one aspect, there is provided a method to form EO, comprising brominating ethylene in an aqueous medium comprising metal bromide with metal ion in higher oxidation state and salt to result in one or more products comprising between about 5-99.9 wt % BE, and the metal bromide with the metal ion in lower oxidation state; and reacting the BE with a base to form EO and brine in water, wherein the reaction forms between about 0-42 tonnes of brine per tonne of EO or any other range of brine per tonne of EO as provided herein.

In one aspect, there is provided a method to form PO, comprising brominating propylene in an aqueous medium comprising metal bromide with metal ion in higher oxidation state and salt to result in one or more products comprising DBP and PBH, and the metal bromide with the metal ion in lower oxidation state; extracting the DBP and the PBH with re-circulating DBP from the same process and/or the other DBP; hydrolyzing the DBP in the mixture of the DBP and the PBH to the PBH and propanal; and reacting the PBH and propanal in presence of remaining DBP with a base to form PO, unreacted propanal, and brine. In one aspect, there is provided a method to form EO, comprising brominating ethylene in an aqueous medium comprising metal bromide with metal ion in higher oxidation state and salt to result in one or more products comprising DBE and BE, and the metal bromide with the metal ion in lower oxidation state; extracting the DBE and the BE with re-circulating DBE from the same process and/or the other DBE; hydrolyzing the DBE in the mixture of the DBE and the BE to the BE and bromoacetaldehyde; and reacting the BE in presence of remaining DBE with a base to form EO, unreacted bromoacetaldehyde, and brine.

In some embodiments of the foregoing aspect, the reaction forms between about 0-42 or about 0-40 tonnes of brine per tonne of PO or EO. In some embodiments of the foregoing aspect, the selectivity of the PBH or the BE formed (after bromination and hydrolysis) is between about 10-99.9 wt %. In some embodiments of the foregoing aspect and embodiments, the base is between about 5-35 wt % or between about 8-25 wt %. The bases have been described herein and include without limitation, the alkali metal hydroxide e.g. sodium hydroxide or potassium hydroxide; alkali earth metal hydroxide e.g. calcium hydroxide or oxide e.g. CaO or MgO; or metal hydroxide bromide. The PO and EO formation has been illustrated in FIGS. 1A, 1B, 3A, 3B, 4A, 4B, 5A, 5B, 6A, and 6B.

In some embodiments of the aforementioned aspects, the PO or the EO formed is between about 5-50 wt %; or between about 5-40 wt %; or between about 5-30 wt %; or between about 5-20 wt %; or between about 5-10 wt %; or between about 10-50 wt %; or between about 10-40 wt %; or between about 10-30 wt %; or between about 10-20 wt %; or between about 20-50 wt %; or between about 20-40 wt %; or between about 20-30 wt %; or between about 30-50 wt %; or between about 30-40 wt %; or between about 40-50 wt %. In some embodiments of the aspects and embodiments provided herein, the PO or the EO formed is between about 1-25 wt %; or between about 2-20 wt %; or between about 3-15 wt %.

In some embodiments of the aspect and embodiments provided herein, the reaction forms between about 0-42 tonnes of brine per tonne of PO or EO; or between about 0-40 tonnes of brine per tonne of PO or EO; or between about 0-35 tonnes of brine per tonne of PO or EO; or between about 0-30 tonnes of brine per tonne of PO or EO; or between about 0-25 tonnes of brine per tonne of PO or EO; or between about 0-20 tonnes of brine per tonne of PO or EO; or between about 0-10 tonnes of brine per tonne of PO or EO; or between about 0-5 tonnes of brine per tonne of PO or EO; or between about 0-4 tonnes of brine per tonne of PO or EO; or between about 0-3 tonnes of brine per tonne of PO or EO; or between about 0-2 tonnes of brine per tonne of PO or EO; or between about 0-1 tonnes of brine per tonne of PO or EO; or between about 3-42 tonnes of brine per tonne of PO or EO; or between about 3-40 tonnes of brine per tonne of PO or EO; or between about 3-35 tonnes of brine per tonne of PO or EO; or between about 3-30 tonnes of brine per tonne of PO or EO; or between about 3-25 tonnes of brine per tonne of PO or EO; or between about 3-20 tonnes of brine per tonne of PO or EO; or between about 3-10 tonnes of brine per tonne of PO or EO; or between about 3-5 tonnes of brine per tonne of PO or EO; or between about 3-4 tonnes of brine per tonne of PO or EO; or between about 5-42 tonnes of brine per tonne of PO or EO; or between about 5-40 tonnes of brine per tonne of PO or EO; or between about 5-35 tonnes of brine per tonne of PO or EO; or between about 5-30 tonnes of brine per tonne of PO or EO; or between about 5-25 tonnes of brine per tonne of PO or EO; or between about 5-20 tonnes of brine per tonne of PO or EO; or between about 5-10 tonnes of brine per tonne of PO or EO. In some embodiments of the aspect and embodiments provided herein, the reaction forms between about 0-40 tonnes of brine per tonne of PO or EO; or between about 0-20 tonnes of brine per tonne of PO or EO; or between about 0-12 tonnes of brine per tonne of PO or EO; or between about 0-4 tonnes of brine per tonne of PO or EO. The “brine” as used herein is same as saltwater.

In some embodiments of the aspect and embodiments provided herein, the base is between about 5-50 wt %; or between about 5-40 wt %; or between about 5-30 wt %; or between about 5-20 wt %; or between about 5-10 wt %; or between about 10-50 wt %; or between about 10-40 wt %; or between about 10-30 wt %; or between about 10-20 wt %; or between about 20-50 wt %; or between about 20-40 wt %; or between about 20-30 wt %; or between about 30-50 wt %; or between about 30-40 wt %; or between about 40-50 wt %; or between about 8-15 wt %; or between about 10-15 wt %; or between about 12-15 wt %; or between about 14-15 wt %; or between about 8-10 wt %; or between about 8-12 wt %. In some embodiments of the aspect and embodiments provided herein, the base is between about 5-38 wt %; or between about 7-33 wt %; or between about 8-20 wt %. In some embodiments, the base concentration is optimized so that the resulting brine concentration is matched to the requirements for the electrochemical system.

In some embodiments, the reactor and/or separator components in the systems of the invention may include a control station, configured to control the amount of propylene or ethylene introduced into the bromination reactor, the amount of the anode electrolyte introduced into the bromination or the oxybromination reactor, the amount of the water containing the organics and the metal ions into the separator, the temperature and pressure conditions in the reactor and the separator, the flow rate in and out of the reactor and the separator, the time and the flow rate of the water going back to the electrochemical cell, etc.

The control station may include a set of valves or multi-valve systems which are manually, mechanically or digitally controlled, or may employ any other convenient flow regulator protocol. In some instances, the control station may include a computer interface, (where regulation is computer-assisted or is entirely controlled by computer) configured to provide a user with input and output parameters to control the amount and conditions, as described above.

The methods and systems of the invention may also include one or more detectors configured for monitoring the flow of propylene or ethylene or the concentration of the metal ion in the aqueous medium/water/saltwater or the concentration of the organics in the aqueous medium/water/saltwater, etc. Monitoring may include, but is not limited to, collecting data about the pressure, temperature and composition of the aqueous medium and gases. The detectors may be any convenient device configured to monitor, for example, pressure sensors (e.g., electromagnetic pressure sensors, potentiometric pressure sensors, etc.), temperature sensors (resistance temperature detectors, thermocouples, gas thermometers, thermistors, pyrometers, infrared radiation sensors, etc.), volume sensors (e.g., geophysical diffraction tomography, X-ray tomography, hydroacoustic surveyers, etc.), and devices for determining chemical makeup of the aqueous medium or the gas (e.g, IR spectrometer, NMR spectrometer, UV-vis spectrophotometer, high performance liquid chromatographs, inductively coupled plasma emission spectrometers, inductively coupled plasma mass spectrometers, ion chromatographs, X-ray diffractometers, gas chromatographs, gas chromatography-mass spectrometers, flow-injection analysis, scintillation counters, acidimetric titration, and flame emission spectrometers, etc.).

In some embodiments, detectors may also include a computer interface which is configured to provide a user with the collected data about the aqueous medium, metal ions and/or the products. For example, a detector may determine the concentration of the aqueous medium, metal ions and/or the products and the computer interface may provide a summary of the changes in the composition within the aqueous medium, metal ions and/or the products over time. In some embodiments, the summary may be stored as a computer readable data file or may be printed out as a user readable document.

In some embodiments, the detector may be a monitoring device such that it can collect real-time data (e.g., internal pressure, temperature, etc.) about the aqueous medium, metal ions and/or the products. In other embodiments, the detector may be one or more detectors configured to determine the parameters of the aqueous medium, metal ions and/or the products at regular intervals, e.g., determining the composition every 1 minute, every 5 minutes, every 10 minutes, every 30 minutes, every 60 minutes, every 100 minutes, every 200 minutes, every 500 minutes, or some other interval.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Various modifications of the invention in addition to those described herein will become apparent to those skilled in the art from the foregoing description and accompanying figures. Such modifications fall within the scope of the appended claims. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

In the examples and elsewhere, some of the abbreviations have the following meanings:

| | | |
|---|---|---|
| AEM | = | anion exchange membrane |
| g | = | gram |
| g/L | = | gram/liter |
| h or hr | = | hour |
| l or L | = | liter |
| M | = | molar |
| kA/m² | = | kiloamps/meter square |
| mg | = | milligram |
| min | = | minute |
| ml | = | milliliter |
| mmol | = | millimole |
| mV | = | millivolt |
| n/kg | = | moles per kilogram |
| μl | = | microliter |
| psi | = | pounds per square inch |
| psig | = | pounds per square inch guage |
| rpm | = | revolutions/minute |
| STY | = | space time yield |
| V | = | voltage |

### EXAMPLES

### Example 1

### Formation of DBP and PBH from Propylene Using Copper Bromide

The experiment was conducted in a 450 mL stirred pressure vessel which contained an inlet for delivering propylene gas and a teflon inner-jacket containing the reactant, i.e. metal bromide solution. A 130 mL solution of 0.4M CuBr, 1.2M CuBr₂, and 0.7M NaBr was placed into the stirred pressure vessel. After purging the closed container with N₂, it was heated to 110° C. After reaching this temperature, 190 psig of propylene was delivered to the vessel and the vessel was stirred. After 5 minutes, stirring was stopped and the vessel was cooled to 30° C., depressurized, and opened. Water was used to rinse the reactor parts and then dibromomethane (DBM) was used as the extraction solvent. The product was analyzed by gas chromatography which showed 2.34 g of DBP and 1.39 g of PBH recovered in the DBM phase.

### Example 2

### Oxybromination Reaction with Varying Cu(I) Concentrations

This example illustrates oxybromination of the metal bromide from the lower oxidation state to the higher oxidation state. Various anolyte compositions shown in Table I below were pipetted into glass vials with magnetic stir bars and split-septa lids.

TABLE I
Initial Compositions

| Sample | 1 | 2 |
|---|---|---|
| Cu(I) [M] | 0.9 | 0.6 |
| Cu(II) [M] | 2.1 | 1.1 |
| NaBr [M] | 1.4 | 1.4 |
| HBr [M] | 0.9 | 0.9 |

For Cu(I) and Cu(II), the initial materials were CuBr and CuBr₂ respectively. The compositions were then oxidized in a parallel high-throughput reactor system. The reaction atmosphere was clean, dry air at a pressure of 90 psig and the reaction temperature was approximately 90° C. Reaction time was 30 minutes. After the reaction was completed, the reaction contents were cooled to ambient temperature and the resulting solutions were titrated for Cu(I) by potentiometric techniques. Sample 1 reacted 0.36M Cu(I) to Cu(II) and Sample 2 reacted 0.56M Cu(I) to Cu(II). Differences could arise owing to differences in density, viscosity, mass transfer with internal stir bar, metal bromide content, etc.

### Example 3

### Epoxidation Reaction to Produce Propylene Oxide with Sodium Hydroxide

This example illustrates the formation of propylene oxide from the epoxidation of propylene bromohydrin. The reaction was conducted in a high-throughput system with a glass vial with a magnetic stir bar and split septa lid. 4 mL of DBP containing 30 g/L PBH was added to the glass vial. The vial was heated to 90° C. 300 μL of 3.1M NaOH in deionized water was added to the glass vial via the split septa. The reaction was stirred at 600 rpm and held at a temperature of 90° C. The reaction time was one minute. After one minute, the glass vial was removed from the high-throughput reactor system and rapidly cooled to room temperature. When the glass vial reached room temperature, the organic phase was sampled and analyzed via gas chromatography. The gas chromatography analysis showed 0.81 mmol, or 98% of the PBH was consumed. 0.80 mmol of propylene oxide was recovered via gas chromatography, resulting in selectivity to propylene oxide of 98%.

### Example 4

### Hydrolysis of DBP to PBH

This example illustrates conversion of DBP to PBH. Various anolyte compositions shown in Table II were pipetted into glass vials in duplicate.

TABLE II
Anolyte compositions

| Anolyte | 1 | 2 | 3 | 4 |
|---|---|---|---|---|
| CuBr₂ (g) | 0.88 | 0.87 | 1.42 | 1.4 |
| CuBr (g) | 0.06 | 0.14 | 0.06 | 0.15 |
| H₂O (g) | 2.95 | 2.84 | 2.7 | 2.62 |

3 mL DBP was added to 3 mL anolyte solution in each vial and a stir bar was added into each vial and capped under argon. After that, the glass vials were transferred to a high-throughput reactor system. The reaction was conducted at 130° C. for 30 minutes. After the reaction, high-throughput reactor cooled down to room temperature. All vials were then diluted with 1,2-dichloroethane solvent to extract organics from the aqueous solution. Both the organic and aqueous phases were analyzed by gas chromatography with all aqueous solutions diluted by acetonitrile before injection. Gas chromatography results for PBH production are shown in the Table III:

TABLE III
PBH amounts in gas chromatography

| Compound | 1 | 2 | 3 | 4 |
|---|---|---|---|---|
| PBH (g) | 21.2 | 22.8 | 23.6 | 25.8 |

### Example 5

### Hydrolysis of DBP to PBH in Absence of Metal Bromide

The water and DBP were added to a series of vials in the amounts shown in Table IV below. The vials were placed inside a multi-channel high-throughput type reactor were they were stirred at approximately 160° C. for 30 minutes. The reactor was quickly cooled and the aqueous and organic phases were analyzed by gas chromatography. The DBP had converted to 1-bromo-2-propanol (PBH), 2-bromo-1-propanol (PBH), propanal, acetone and other byproducts. The yields of the desired products are shown in Table IV below. It was observed that higher organic:aqueous ratio resulted in lower yield of the hydrolyzed products. Due to the absence of the metal bromine, no further brominated products from propanal or acetone were observed.

TABLE IV
Hydrolysis products

| Nominal | | | | | | |
|---|---|---|---|---|---|---|
| Ratio | | | 1-bromo-2- | 2-bromo-1- | | |
| DBP:Water | DBP | Water | propanol | propanol | Propanal | Acetone |
| (vol/vol) | (grams) | (grams) | (mg) | (mg) | (mg) | (mg) |
| 29:1 | 5.43 | 0.10 | 1.94 | 0.23 | 0.03 | 0.00 |
| 29:1 | 5.40 | 0.11 | 2.81 | 0.32 | 0.05 | 0.00 |
| 5:1 | 4.66 | 0.50 | 25.80 | 1.35 | 1.03 | 2.20 |
| 5:1 | 4.67 | 0.50 | 25.25 | 1.33 | 1.00 | 2.08 |
| 2:1 | 3.71 | 1.00 | 37.96 | 1.59 | 2.21 | 7.73 |
| 2:1 | 3.58 | 1.00 | 38.82 | 1.60 | 2.28 | 7.79 |
| 1:1 | 2.82 | 1.49 | 41.87 | 1.48 | 3.19 | 14.52 |
| 1:1 | 2.74 | 1.48 | 42.01 | 1.55 | 3.18 | 16.13 |

### Example 6

### Hydrolysis of DBP to PBH in Presence of Metal Bromide

An aqueous solution containing 1.6 mol/kg of CuBr₂, 0.2 mol/kg of CuBr, and 0.5 mol/kg of NaBr with the balance being DI water was placed into 8 separate high throughput vials. Varying amounts of DBP were then added to the copper containing solution so that the organic:aqueous ratios were 0.5:1, 1:1, 1.5:1, and 2:1 and the overall solution volume was nominally the same (6 mL) with each ratio run in duplicate. The vials were then placed into a high throughput clamshell reactor and heated at 130° C. for 30 minutes. The vials were then removed and analyzed by Gas Chromatography. The products observed included 2-bromopropanal, 2,2-dibromopropranal, 1-bromoacetone, and 1,1-dibromoacetone. Using estimated response factors for the various brominated propanal compounds and brominated acetone compounds, the yields were calculated and tabulated as shown below in Table V:

TABLE V
Hydrolysis products

| | | | | 2,2- | | 1,1- |
|---|---|---|---|---|---|---|
| | 1-bromo-2- | 2-bromo-1- | 1-bromo | dibromo | 1-bromo | dibromo |
| Nominal Ratio | propanol | propanol | propanal | propanal | acetone | acetone |
| DBP:Aqeuous | μmol | μmol | μmol | μmol | μmol | μmol |
| 0.5:1 | 290.9 | 4.6 | 19.3 | 2.96 | 14 | 13.6 |
| 0.5:1 | 288.5 | 4.4 | 18.6 | 3.16 | 13.2 | 13.1 |
| 1:1 | 233.1 | 6.5 | 14.4 | 1.38 | 10.5 | 5.4 |
| 1:1 | 244.5 | 6.6 | 15.5 | 1.44 | 10.9 | 5.6 |
| 1.5:1 | 198.7 | 7.4 | 11.4 | 0.85 | 7.8 | 2.9 |
| 1.5:1 | 210.8 | 7.9 | 11.8 | 0.72 | 8.5 | 3.2 |
| 2:1 | 180.3 | 7.7 | 9.1 | 0.51 | 6.6 | 1.7 |
| 2:1 | 172.4 | 8.1 | 9.1 | 0.49 | 5.6 | 1.9 |

### Example 7

### Hydrolysis of DBE to BE

An 8-well pressure vessel was pre-heated to 150° C. on a stirring hotplate. Meanwhile, four anolytes were prepared in duplicate with CuBr, CuBr₂, and NaBr salts dissolved in a deionized water solvent: Anolyte A contained 0.6 n/kg CuBr, 1.8 n/kg CuBr₂, and 0.0 n/kg NaBr; Anolyte B contained 0.6 n/kg CuBr, 1.7 n/kg CuBr₂, and 0.3 n/kg NaBr; Anolyte C contained 0.6 n/kg CuBr, 1.6 n/kg CuBr₂, and 0.4 n/kg NaBr; and Anolyte D contained 0.2 n/kg CuBr, 1.6 n/kg CuBr₂, and 0.5 n/kg NaBr. 2.5 mL of each anolyte was pipetted into a 10 mL vial that also contained 2.5 mL of DBE and a stir-bar. Each vial was then loaded into the pre-heated pressure vessel which was subsequently closed and had approximately 50 psig of nitrogen applied as back-pressure to the 10 mL vials. Reaction time started when the stirring setpoint was set to 600 rpm and ended 15 minutes later. The pressure vessel was immediately cooled externally with ice until the temperature of the pressure vessel dropped to 100° C., after which the pressure vessel was opened, and the vials were cooled individually to room temperature. After the samples reached room temperature, an aliquot of the DBE phase was transferred to a GC vial for GCMS analysis. A 1 mL aliquot of aqueous anolyte was transferred into a separate vial, and each aliquot was extracted with 3 mL of ethyl acetate. The ethyl acetate phase was transferred to a GC vial for GCMS analysis.

The GCMS was calibrated for BE based on TIC area, and the BE response factor was used to estimate the concentration of all other byproducts, as measured by TIC areas. For analysis of the DBE phase, any impurities found in a scan of the unheated DBE were subtracted from the total area count of the corresponding observed byproducts. The procedure described above yielded 0.47-0.60 mmol of organics. The overall selectivity to BE and tribromoacetaldehyde (bromal) ranged from 73%-80% and 10%-13%, respectively. 72%-77% of the measured BE and 96%-97% of the measured tribromoacetaldehyde were recovered in the DBE phase. The overall selectivity to dibromomethane was 4%-7%. Other compounds that each comprised 1%-3% of the observed byproducts by GCMS included bromomethane, dibromomethane, tribromomethane, bromoethane, and tribromoethane. Results were similar for all four anolyte compositions. See Table VI for details.

TABLE VI
Hydrolysis products

| Nominal | | | | | | Tri- | | Tri- |
|---|---|---|---|---|---|---|---|---|
| Volumetric | Anolyte | | Tribromo | Bromo | Dibromo | bromo | Bromo | bromo |
| Ratio | Name | BE | acetaldehyde | methane | methane | methane | ethane | ethane |
| DBE:Aqueous | A, B, C, D | μmol | μmol | μmol | μmol | μmol | μmol | μmol |
| 1:1 | A | 450 | 65 | 7 | 34 | 4 | 17 | 13 |
| 1:1 | A | 406 | 74 | 7 | 36 | 4 | 5 | 13 |
| 1:1 | B | 436 | 67 | 7 | 36 | 4 | 16 | 13 |
| 1:1 | B | 401 | 71 | 8 | 34 | 4 | 7 | 12 |
| 1:1 | C | 424 | 51 | 7 | 27 | 3 | 4 | 9 |
| 1:1 | C | 387 | 58 | 7 | 30 | 3 | 6 | 9 |
| 1:1 | D | 391 | 56 | 6 | 21 | 3 | 2 | 11 |
| 1:1 | D | 362 | 59 | 6 | 20 | 4 | 3 | 9 |

What is claimed is:

1. A method, comprising:
brominating propylene with an aqueous medium comprising metal bromide with metal ion in higher oxidation state, metal bromide with metal ion in lower oxidation state, and saltwater to result in one or more products comprising dibromopropane (DBP) and propylenebromohydrin (PBH) and reduction of the metal bromide with the metal ion in the higher oxidation state to the metal bromide with the metal ion in the lower oxidation state;
epoxidizing the one or more products comprising DBP and PBH with a base to form propylene oxide (PO) and unreacted DBP; and
subjecting the unreacted DBP to hydrolysis under one or more reaction conditions to result in hydrolysis products comprising PBH and propanal.

2. The method of claim 1, wherein the one or more reaction conditions in the hydrolysis reaction comprise organic:aqueous ratio between 0.5:10-10:0.5.

3. The method of claim 1, wherein the one or more reaction conditions in the hydrolysis reaction comprise Lewis acid selected from the group consisting of silicon bromide; germanium bromide; tin bromide; boron bromide; aluminum bromide; gallium bromide; indium bromide; thallium bromide; phosphorus bromide; antimony bromide; arsenic bromide; copper bromide; zinc bromide; titanium bromide; vanadium bromide; chromium bromide; manganese bromide; iron bromide; cobalt bromide; nickel bromide; lanthanide bromide; and triflate.

4. The method of claim 1, further comprising separating the one or more products comprising PBH and DBP from the aqueous medium, before subjecting the one or more products comprising PBH and DBP to the epoxidation reaction.

5. The method of claim 1, further comprising, without separating subjecting the aqueous medium comprising metal bromide with metal ion in higher oxidation state, metal bromide with metal ion in lower oxidation state, and saltwater, and the one or more products comprising PBH and DBP, to hydrolysis reaction before the epoxidation reaction.

6. The method of claim 1, wherein the hydrolysis products further comprise bromopropanal, dibromopropanal, acetone, bromoacetone, dibromoacetone, unreacted DBP, or combinations thereof.

7. The method of claim 1, further comprising circulating the hydrolysis products comprising PBH and propanal from the hydrolysis reaction back to the epoxidation reaction to form the PO, the unreacted DBP, unreacted propanal, or combinations thereof.

8. The method of claim 7, further comprising separating the PO from the unreacted propanal.

9. The method of claim 1, wherein the base comprises alkali metal hydroxide and/or alkali earth metal hydroxide.

10. The method of claim 1, wherein reaction conditions for the bromination reaction comprise temperature of the reaction between 40-120° C.; concentration of the metal bromide with metal ion in the higher oxidation state entering the bromination to be between 0.5-3M; concentration of the metal bromide with metal ion in the lower oxidation state entering the bromination to be between 0.01-2M; or combinations thereof.

11. The method of claim 1, further comprising, before the bromination, contacting an anode with an anode electrolyte in an electrochemical cell wherein the anode electrolyte comprises metal bromide with metal ion in higher oxidation state, metal bromide with metal ion in lower oxidation state, and saltwater; contacting a cathode with a cathode electrolyte in the electrochemical cell; applying voltage to the anode and the cathode and oxidizing the metal bromide with the metal ion in the lower oxidation state to the higher oxidation state at the anode; and transferring the anode electrolyte from the electrochemical cell to the bromination reaction.

12. The method of claim 11, further comprising forming sodium hydroxide or potassium hydroxide in the cathode electrolyte and using the sodium hydroxide or the potassium hydroxide as the base to form the PO.

13. The method of claim 1, further comprising, after the bromination, oxybrominating the metal bromide with the metal ion in the lower oxidation state to the higher oxidation state in presence of oxygen and optionally HBr.

14. The method of claim 13, further comprising recirculating the metal bromide with the metal ion in the higher oxidation state back to the bromination reaction and/or back to an anode electrolyte of an electrochemical cell.

15. The method of claim 13, wherein reaction conditions for the oxybromination reaction comprise temperature between about 50-100° C.; pressure between about 1-100 psig; oxygen partial pressure in feed to the oxybromination in a range between about 0.01-100 psia; or combinations thereof.

16. The method of claim 1, wherein the saltwater is an alkali metal bromide selected from the group consisting of sodium bromide, potassium bromide, lithium bromide, and combinations thereof, or alkali earth metal bromide selected from the group consisting of calcium bromide, strontium bromide, magnesium bromide, and combinations thereof.

17. The method of claim 1, wherein yield of the PO is more than 80 wt % and/or space time yield (STY) of the PO is more than 0.1 (mol/L/hr).

18. The method of claim 1, wherein the metal bromide with the metal ion in the lower oxidation state is CuBr and the metal bromide with the metal ion in the higher oxidation state is CuBr₂.

19. A system, comprising:
a bromination reactor configured to receive an aqueous medium comprising metal bromide with metal ion in higher oxidation state, metal bromide with metal ion in lower oxidation state, and saltwater and brominate propylene with the metal bromide with the metal ion in the higher oxidation state to result in one or more products comprising PBH and DBP, and the metal bromide with the metal ion in the lower oxidation state;
an epoxide reactor operably connected to the bromination reactor and configured to receive the one or more products comprising PBH and DBP and epoxidize with a base to form PO and unreacted DBP; and
a hydrolysis reactor operably connected to the epoxide reactor and configured to receive the unreacted DBP from the epoxide reactor and hydrolyze under one or more reaction conditions to result in hydrolysis products comprising PBH and propanal.

20. The system of claim 19, further comprising an electrochemical cell operably connected to the bromination reactor, the hydrolysis reactor, and/or the epoxide reactor, comprising an anode in contact with an anode electrolyte wherein the anode electrolyte comprises metal bromide with metal ion in higher oxidation state, metal bromide with metal ion in lower oxidation state, and saltwater; a cathode in contact with a cathode electrolyte; and a voltage source configured to apply voltage to the anode and the cathode wherein the anode is configured to oxidize the metal bromide with the metal ion from the lower oxidation state to the higher oxidation state; and/or further comprising an oxybromination reactor operably connected to the electrochemical cell and/or the bromination reactor and configured to oxybrominate the metal bromide with the metal ion from the lower oxidation state to the higher oxidation state in presence of HBr and oxygen.
